(19) United States
(12) Patent Application Publication
(10) Pub. No.: US 2023/0066762 A1
(43) Pub. Date: Mar. 2, 2023

(54) IMMUNOGENIC COMPOSITION

(71) Applicant: GLAXOSMITHKLINE BIOLOGICALS SA, Rixensart (BE)

(72) Inventors: Ghislain DELPIERRE, Rixensart (BE); Juliette FORTPIED, Rixensart (BE); Virginie HELLEBAUT, Rixensart (BE); Vincent Edwin Paul LEVET, Rixensart (BE); Roland MAINIL, Rixensart (BE); Frédéric Stéphane MATHOT, Rixensart (BE); Maria Dolores MORALES AIRA, Rixensart (BE); Philippe SCIEUR, Rixensart (BE); Bram VUYLSTEKE, Rixensart (BE)

(73) Assignee: GLAXOSMITHKLINE BIOLOGICALS SA, Rixensart (BE)

(21) Appl. No.: 17/632,299

(22) PCT Filed: Aug. 3, 2020
(86) PCT No.: PCT/EP2020/071760

§ 371 Date: Feb. 2, 2022

(30) Foreign Application Priority Data
Aug. 5, 2019 (EP) 19189963.2

(51) Int. Cl. A61K 39/145; A61K 39/104; A61K 47/20; A61K 47/10; A61K 47/26

(52) U.S. Cl. CPC A61K 39/145; A61K 39/1045; A61K 47/20; A61K 47/10; A61K 47/26; A61K 2039/55555

(57) ABSTRACT

The present invention relates to immunogenic compositions comprising immunogenic polypeptides from Haemophilus influenzae and Moraxella catarrhalis and their use in the treatment or prevention of an acute exacerbation of chronic obstructive pulmonary disease (AECOPD) in a subject, e.g. human.

### TECHNICAL FIELD

The present invention relates to immunogenic compositions and the use of such compositions in medicine. More particularly, it relates to immunogenic compositions comprising immunogenic polypeptides from Haemophilus influenzae and Moraxella catarrhalis and their use in the treatment or prevention of an acute exacerbation of chronic obstructive pulmonary disease (AECOPD) in a subject, e.g. human.

### BACKGROUND TO THE INVENTION

Chronic Obstructive Pulmonary Disease (COPD) is a chronic inflammatory disorder resulting in irreversible decline in lung function as a consequence of inhalation of tobacco smoke or other irritants. Chronic obstructive pulmonary disease (COPD) is recognised as encompassing several conditions (airflow obstruction, chronic bronchitis, bronchiolitis or small airways disease and emphysema) that often coexist (Wilson et al., Eur. Respir. J. 2001; 17: 995-1007). Patients suffer exacerbations of their condition that are usually associated with increased breathlessness, and often have increased cough that may be productive of mucus or purulent sputum (Wilson, Eur Respir J 2001 17:995-1007). COPD is defined physiologically by the presence of irreversible or partially reversible airway obstruction in patients with chronic bronchitis and/or emphysema (Standards for the diagnosis and care of patients with chronic obstructive pulmonary disease. American Thoracic Society. Am J Respir Crit Care Med. 1995 November; 152 (5 Pt 2):S77-121).

COPD is a major cause of morbidity and mortality worldwide. Approximately one in 20 deaths in 2005 in the US had COPD as the underlying cause (Drugs and Aging 26:985-999 (2009)). It is projected that in 2020 COPD will rise to the fifth leading cause of disability adjusted life years, chronic invalidating diseases, and to the third most important cause of mortality (Lancet 349:1498-1504 (1997)).

The course of COPD is characterized by progressive worsening of airflow limitation and a decline in pulmonary function. COPD may be complicated by frequent and recurrent acute exacerbations (AE), which are associated with enormous health care expenditure and high morbidity (Proceedings of the American Thoracic Society 4:554-564 (2007)). One study suggests that approximately 50% of acute exacerbations of symptoms in COPD are caused by non-typeable Haemophilus influenzae, Moraxella catarrhalis, Streptococcus pneumoniae, and Pseudomonas aeruginosa. (Drugs and Aging 26:985-999 (2009)). Haemophilus influenzae (H. influenzae) is found in 20-30% of exacerbations of COPD; Streptococcus pneumoniae, in 10-15% of exacerbations of COPD; and Moraxella catarrhalis, in 10-15% of exacerbations of COPD (New England Journal of Medicine 359:2355-2365 (2008)). Haemophilus influenzae, Streptococcus pneumoniae, and Moraxella catarrhalis have been shown to be the primary pathogens in acute exacerbations of bronchitis in Hong Kong, South Korea, and the Phillipines, while Klebsiella spp., Pseudomonas aeruginosa and Acinetobacter spp. constitute a large proportion of pathogens in other Asian countries/regions including Indonesia, Thailand, Malaysia and Taiwan (Respirology, (2011) 16, 532-539; doi:10.1111/j.1440.1843.2011.01943.x). In Bangladesh, 20% of patients with COPD showed positive sputum culture for Pseudomonas, Klebsiella, Streptococcus pneumoniae and Haemophilus influenzae, while 65% of patients with AECOPD (acute exacerbation of COPD) showed positive cultures for Pseudomonas, Klebsiella, Acinetobacter, Enterobacter, Moraxella catarrhalis and combinations thereof. (Mymensingh Medical Journal 19:576-585 (2010)). However, it has been suggested that the two most important measures to prevent COPD exacerbation are active immunizations and chronic maintenance of pharmacotherapy (Proceedings of the American Thoracic Society 4:554-564 (2007)).

One of the difficulties in treating and managing COPD is the heterogeneity of this complex disease in terms of severity, progression, exercise tolerance, and nature of symptoms. This complexity is also evident in acute exacerbations of COPD (AECOPD), which are transient and apparently stochastic periods of increased COPD symptoms requiring additional medical treatment and often hospitalization (Sethi et al., N Eng J Med 2008; 359:2355-65). Known subtypes of exacerbations are defined by the nature of key triggers including bacterial or viral infections, and/or high eosinophil levels, and these events are typically treated with a combination of antibiotics and steroids in a non-specific manner (Bafadhel et al., Am J Respir Crit Care Med 2011; 184:662). A Protein D polypeptide from Haemophilus influenzae together with a PE-PilA fusion protein and an UspA2 polypeptide from Moraxella catarrhalis is proposed as a vaccine in the treatment or prevention of acute exacerbations of COPD (AECOPD), as described in WO2015125118A1.

There exists a need for improved immunogenic compositions. In particular, there is a need for improved immunogenic compositions to help maintain the structure and function of protein antigens. Such considerations include, but are not limited to, chemical stability of the immunogenic composition (e.g. proteolysis or fragmentation of proteins), physical/thermal stability of the immunogenic composition (e.g., aggregation, precipitation, adsorption), compatibility of the immunogenic composition with the container/closure system, interactions between immunogenic composition and inactive ingredients (e.g. buffers, salts, excipients, cryoprotectants), the manufacturing process, the dosage form (e.g., lyophilized, liquid), the environmental conditions encountered during shipping, storage and handling (e.g., temperature, humidity, shear forces), and the length of time between manufacture and usage.

Consistency and shelf life of biological medicaments can be affected by oxidation during the manufacturing process, or during long term storage, or from process steps such as freezing, drying and freeze drying, or from a combination of these factors. Oxidation from exposure to air or to reagents or conditions used in manufacture, for example hydrogen peroxide used to sterilise equipment may be responsible. A lyophilisation process used to freeze dry many vaccines or other biological medicaments, may also be responsible or may exacerbate the problem, for example through cryocentration of components of the medicament. Proteins can be targeted for oxidation both at the protein backbone, which can result in fragmentation of the back bone, and on the amino acid side chains. Oxidation of the side chains can lead to conformational changes and dimerization or aggregation. Oxidation can thus result in protein damage and can have serious consequences for the structure and function of the proteins. The side chains of cysteine, methionine, tryptophan, histidine and tyrosine are major targets for oxidation, in that order (Ji et al 2009, J Pharmaceutical Sciences, Vol 98, No 12, 4485-4500). The ease of oxidation of sulphur centres makes cysteine and methionine residues preferred sites for oxidation within proteins.

There is a need for improved immunogenic compositions comprising protein antigens: Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide and an UspA2 polypeptide. For example, there is a need for immunogenic compositions which (i) reduce aggregation of the protein antigens, and/or (ii) reduce oxidation of the protein antigens, and/or (iii) have improved stability. Whilst immunogenic compositions comprising an immunogenic polypeptides from Haemophilus influenzae and Moraxella catarrhalis are described in WO2018178264A1, the identification of protein antigens sensitive to aggregation, oxidation and/or destabilisation and the use of certain excipients, in particular the combination of certain excipients, to allieviate such issues has not previously been addressed.

### SUMMARY OF THE INVENTION

The present invention provides immunogenic compositions which (i) reduce aggregation of protein antigens (in particular aggregation caused by shear stress) and/or (ii) reduce oxidation of protein antigens (in particular oxidation of methionine residues) and thus help maintain the structure and function of the protein antigens. The present inventors have identified protein antigens sensitive to aggregation, oxidation and/or destabilisation and provide immunogenic compositions to improve the stability of such protein antigens in the immunogenic composition, which may thus help maintain the immunogenicity of the protein antigens. The immunogenic compositions of the present invention comprise an antioxidant (e.g. L-methionine) and poloxamer (e.g. poloxamer 188, also referred to as “PX188”). According to the present invention it was found that adding an antioxidant (e.g. L-methionine) and poloxamer (e.g poloxamer 188) to immunogenic compositions comprising protein antigens: Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide and an UspA2 polypeptide provides an improved immunogenic composition. Thus, a first aspect of the invention is an immunogenic composition combining an antioxidant (e.g. L-methionine) and poloxamer (e.g. poloxamer 188). It has also surprisingly been found that the stability of protein antigen in the immunogenic composition can be still further improved by the addition of polysorbate 80 (also referred to as “PS80”), even in a residual amount. Thus adding an antioxidant (e.g. L-methionine), poloxamer (e.g poloxamer 188) and polysorbate 80 to immunogenic compositions comprising protein antigens: Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide and an UspA2 polypeptide provides a further improved immunogenic composition. Thus, a second aspect of the invention is an immunogenic composition comprising an antioxidant (e.g. L-methionine), poloxamer (e.g. poloxamer 188) and polysorbate 80. Accordingly, the present invention provides an immunogenic composition comprising Protein E from Haemophilus influenzae or an immunogenic fragment thereof and PilA from Haemophilus influenzae or an immunogenic fragment thereof, optionally as a fusion protein (optionally a PE-PilA fusion protein, e.g. SEQ ID NO: 9); a Protein D polypeptide (optionally a Protein D polypeptide of SEQ ID NO: 2); an UspA2 polypeptide (optionally an UspA2 polypeptide of SEQ ID NO: 19); an anti-oxidant (optionally L-methionine); and poloxamer (optionally poloxamer 188). The present invention also provides an immunogenic composition comprising Protein E from Haemophilus influenzae or an immunogenic fragment thereof and PilA from Haemophilus influenzae or an immunogenic fragment thereof, optionally as a fusion protein (optionally a PE-PilA fusion protein, e.g. SEQ ID NO: 9); a Protein D polypeptide (optionally a Protein D polypeptide of SEQ ID NO: 2); an UspA2 polypeptide (optionally an UspA2 polypeptide of SEQ ID NO: 19); an anti-oxidant (optionally L-methionine); poloxamer (optionally poloxamer 188) and polysorbate 80.

The present invention also provides a process for preparing an immunogenic composition of the invention.

The present invention also provides a kit comprising a first container comprising an immunogenic composition of the invention and a second container comprising an adjuvant.

The present invention also provides an immunogenic composition of the invention, for use in the treatment or prevention of an acute exacerbation of COPD (AECOPD) in a subject, e.g. human.

The present invention also provides the use of an immunogenic composition of the invention, in the manufacture of a medicament for the treatment or prevention of an acute exacerbation of COPD (AECOPD) in a subject, e.g. human.

The present invention also provides a method of treatment of an acute exacerbation of COPD (AECOPD) in a subject, e.g. human, at risk of developing an acute exacerbation of COPD (AECOPD), said method comprising administering to said subject, an effective amount of an immunogenic composition of the invention.

The present invention also provides a method of prevention of an acute exacerbation of COPD (AECOPD) in a subject, e.g. human, at risk of developing an acute exacerbation of COPD (AECOPD), said method comprising administering to said subject, an effective amount of an immunogenic composition of the invention.

### DETAILED DESCRIPTION

### Definitions

As used herein, “adjuvant” means a compound or substance that, when administered to a subject in conjunction with a vaccine, immunotherapeutic, or other antigen- or immunogen-containing composition, increases or enhances the subject's immune response to the administered antigen or immunogen (as compared to the immune response that would be obtained in the absence of adjuvant).

As used herein, the term “immunogenic fragment” is a portion of an antigen smaller than the whole, that is capable of eliciting a humoral and/or cellular immune response in a host animal, e.g. human, specific for that fragment. Thus a fragment of a genomic sequence does not include the genomic sequence itself and a fragment of a protein does not include the full length protein sequence itself.

Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. An immunogenic fragment of the invention may be derived from an amino acid sequence at least 70%, 80%, 85%, 90%, 91%, 92%, 93%, 94%, 95%, 96%, 97%, 98%, 99%, or 100% identical to a reference sequence (e.g. SEQ ID NO: 1 to 58 of the present invention) which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. For an example, an immunogenic fragment may be derived by deletion of the signal peptide.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a reference polypeptide.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein a “subject” is a mammal, including humans, non-human primates, and non-primate mammals such as members of the rodent genus (including but not limited to mice and rats) and members of the order Lagomorpha (including but not limited to rabbits). In particular embodiments, the subject is a human.

As further described below, an acute exacerbation of COPD (AECOPD) is an acute event characterised by a worsening of the patient's respiratory symptoms that is beyond normal day-to-day variations. Typically an AECOPD leads to a change in medication.

As used herein, the term “treatment of an acute exacerbation of COPD (AECOPD)” means ameliorating, stabilising, reducing or eliminating the increased symptoms that are a feature of an acute exacerbation in a subject, e.g. human.

As used herein, the phrase “prevention of an acute exacerbation of COPD (AECOPD)” means preventing, reducing the incidence or frequency, or reducing the severity (e.g. airflow obstruction, chronic bronchitis, bronchiolitis or small airways disease and emphysema) of future acute exacerbations in a subject, e.g. human.

As used herein, the term “treatment of a disease caused by H. influenzae and/or M. catarrhalis” means ameliorating, stabilising, reducing or eliminating the increased symptoms that are a feature of a bacterial infection caused by H. influenzae and/or M. catarrhalis in a subject, e.g. human.

As used herein, the phrase “prevention of a disease caused by H. influenzae and/or M. catarrhalis” means preventing, reducing the incidence or frequency, or reducing the severity of future bacterial infections caused by H. influenzae and/or M. catarrhalis in a subject, e.g. human.

As used herein, the term “bacterial infection” refers to a positive test for a bacterial pathogen on routine culture (Haemophilus influenza or Moraxella catarrhalis) or a total aerobic CFU count greater than or equal to 10⁷ cells. In particular embodiments, the bacterial infection is associated with
a) Haemophilus influenza (e.g. non-typeable H. influenzae (NTHi));
b) Moraxella catarrhalis; or
c) Haemophilus influenzae (e.g. non-typeable H. influenzae (NTHi)) and Moraxella catarrhalis.

As used herein, the term “effective amount” in the context of administering an immunogenic composition or vaccine of the invention to a subject refers to the amount of the immunogenic composition or vaccine which has a prophylactic and/or therapeutic effect. As used herein “w/v” means weight/volume of the formulation.

Identity between polypeptides may be calculated by various algorithms. In general, when calculating percentage identity the two sequences to be compared are aligned to give a maximum correlation between the sequences. This may include inserting “gaps” in either one or both sequences, to enhance the degree of alignment. For example the Needleman Wunsch algorithm (Needleman and Wunsch 1970, J. Mol. Biol. 48: 443-453) for global alignment, or the Smith Waterman algorithm (Smith and Waterman 1981, J. Mol. Biol. 147: 195-197) for local alignment may be used, e.g. using the default parameters (Smith Waterman uses BLOSUM 62 scoring matrix with a Gap opening penalty of 10 and a Gap extension penalty of 1). A preferred algorithm is described by Dufresne et al. in Nature Biotechnology in 2002 (vol. 20, pp. 1269-71) and is used in the software GenePAST (Genome Quest Life Sciences, Inc. Boston, Mass.). The GenePAST “percent identity” algorithm finds the best fit between the query sequence and the subject sequence, and expresses the alignment as an exact percentage. GenePAST makes no alignment scoring adjustments based on considerations of biological relevance between query and subject sequences. Identity between two sequences is calculated across the entire length of both sequences and is expressed as a percentage of the reference sequence (e.g. SEQ ID NOs. 1 to 58 of the present invention). For fragments, the reference sequence is the longest sequence.

### DESCRIPTION OF FIGURES

FIG. 1: Mass spectrometry results for protein D Met192 oxidation over time for 0 and 1300 ng/mL H₂O₂ at different temperatures.

FIG. 2: RP-HPLC chromatogram of protein D (PD) with 1300 ng/mL H₂O₂ stored for 3 days at 45° C. and of non-spiked protein D (without H₂O₂) stored at 4° C., indicating the protein D main peak and oxidized protein D pre-peaks.

FIG. 3: Antigen profiles for protein D (PD), UspA2 and PE-PilA, obtained by SDS-PAGE under non-reducing conditions, without H₂O₂ (left hand lane) or with H₂O₂ (right hand lane), when stored at (i) 4° C. (time=0) (lanes 3 and 4) (ii) 37° C. for 15 days (lanes 5 and 6) and (iii) at 45° C. for 7 days (lanes 7 and 8).

FIG. 4: Mass spectrometry results for protein D Met192 oxidation over time: Oxidised No AOX (with H₂O₂, no antioxidant), Not Oxidised No AOX (without H₂O₂, no antioxidant), Oxidised 30 mM CYS (with H₂O₂ and cysteine (CYS)), Oxidised 50 mM MET (with H₂O₂ and methionine (MET)).

FIG. 5: RP-HPLC chromatogram of protein D (PD) treated with H₂O₂ comparing (i) 18COP1141 without H₂O₂ and without antioxidant, (ii) 18COP1146 without anti-oxidant (AOx), (iii) 18COP1147 with methionine (Meth), and (iv) 18COP1149 with cysteine (Cysteine).

FIG. 6: Antigen profile for UspA2, protein D (PD) and PE-PilA obtained by SDS-PAGE under non-reducing conditions, comparing samples without anti-oxidant (left hand lane), with methionine (MET) (middle lane) or with cysteine (CYS) (right hand land), under conditions: (i) without H₂O₂ (lanes 3 to 5) or (ii) with H₂O₂ (lanes 6 to 8).

FIG. 7: Hydrophobic variants HPLC for a composition containing Protein D, PE-PilA and UspA2, for samples 18COP1401 (no L-Methionine, no H2O2), 18COP1402 (5 mM L-Methionine, H2O2) and 18COP1407 (no L-Methionine, H2O2).

FIG. 8: Hydrophobic variants HPLC for a composition containing Protein D, PE-PilA and UspA2, showing the protein D peak, for sample 18COP1403 with H₂O₂ and 10 mM L-methionine.

FIG. 9: Hydrophobic variants RP-HPLC % peak3, for protein D in a composition containing Protein D, PE-PilA and UspA2; in the left panel non H₂O₂ oxidized samples without antioxidant (“Ref Sample”); in the right panel H₂O₂ oxidized samples with methionine at different concentrations (“Treated (Spiked 1300)”).

FIG. 10: Hydrophobic variants RP-HPLC % peak3, for protein D in a composition containing Protein D, PE-PilA and UspA2, H₂O₂ oxidized samples with methionine at different concentrations from 0 mM methionine (“Ref Sample”) to 50 mM methionine.

FIG. 11: From RP-HPLC, the sum of area of peaks 1, 2 and 3 for protein D in a composition containing Protein D, PE-PilA and UspA: (i) Ref (reference) with no H₂O₂, (ii) positive control with H₂O₂, and (iii) with 10 mM Met and H₂O₂.

FIG. 12: Liquid chromatography coupled mass spectrometry for protein D M192 oxidation in % after 1 month at 37° C. Left panel without H₂O₂ and without methionine (“Ref sample”). Right panel with 1300 ng of H₂O₂ per mL before freeze drying, with or without methionine (“Treated (Spiked 1300)”).

FIG. 13: As FIG. 12, liquid chromatography coupled mass spectrometry for protein D M192 oxidation. Left panel without H₂O₂ and without methionine (“Ref sample”). Right panel with 1300 ng of H₂O₂ per mL added before freeze drying and with 10 mM methionine.

FIG. 14: HPLC (High-performance liquid chromatography) SEC (Size-exclusion chromatography) profiles of UspA2 containing increasing amount of PX188 reconstituted with NaCl.

FIG. 15: Impact of PX188 (Poloxamer 188) on the shear stress resistance of UspA2, comparing different concentrations of poloxamer: PX188 0.001%, PX188 0.005%, PX188 0.01%, PX188 0.02%, PX188 0.05%, PX188 0.1%, PX188 0.15% to PS80 0.05%.

FIG. 16: PE-PilA recovery measured by UPLC (Ultra High Performance Liquid Chromatography): for “CTRL PS80 res.” (Control with residual PS80), “PS80” (0.05% polysorbate 80), and “PX188” (Poloxamer 188) at various concentrations.

FIG. 17: Process Flow Diagrams. Process A—PE-PilA drug substance was prepared with PS80. Process B—PE-PilA drug substance was prepared with poloxamer 188. Process C—new proposed process in which PE-PilA will be prepared with PS80 and the final drug product will be formulated with poloxamer 188 and methionine.

FIG. 18: Comparison of in-use stability at +30° C. of Process A material (COP14303A) and Process B material (19COP0410) reconstituted in adjuvant buffer: HPSEC-fluo profile. UspA2 pre-peak appears in HPSEC profile after reconstitution of Process B drug product in adjuvant buffer, but not after reconstitution of Process A drug product in adjuvant buffer.

FIG. 19: UspA2 content by UPLC decreases after reconstitution of Process B drug product in adjuvant buffer, but not in Process A drug product in adjuvant buffer.

FIG. 20: Process B: UspA2 potency measured by ELISA. No loss of UspA2 potency for drug product reconstituted in AS01E adjuvant.

FIG. 21: Comparison of in-use stability at +30° C. of Process A material (repro lot COP14303A) and Process B material (lot 19COPO410) reconstituted in adjuvant buffer: UspA2 content by UPLC. UspA2 content by UPLC decreases after reconstitution of Process B drug product in adjuvant buffer, but not in Process A drug product in adjuvant buffer.

FIG. 22: Analysis of “crossed” formulations using Drug substance of different sources. Recovery of UspA2 content by UPLC after 24h at 30° C. relative to T0 (i.e. compared to the UspA2 content at the start). The instability of UspA2 in drug product was linked to Process B PE-PilA drug substance (no impact of Methionine or lyophilization cycle).

FIG. 23: HPSEC profile does not evolve after reconstitution in adjuvant or adjuvant buffer for process A. No UspA2 pre-peak for Process B drug product after reconstitution in AS01E adjuvant, however a pre-peak is observed when reconstituting in AS01E buffer.

FIG. 24: UspA2 content recovery after 24h 30° C. by UPLC. Polysorbate 80 spiked in reconstituted vaccine (adjuvant buffer) prevents UspA2 instability. Comparison of the UspA2 content recovery after 24h relative to T0 (i.e. compared to the UspA2 content at the start) for Process A (left) and Process B (right).

FIG. 25: Comparison of the evolution of UspA2 content by UPLC during in-use stability (24h30° C.) after reconstitution in adjuvant buffer, on different lots having different ages. UspA2 content recovery by UPLC. The instability of UspA2 (in adjuvant buffer) is more pronounced with older drug product.

### IMMUNOGENIC COMPOSITIONS

Immunogenic compositions of the invention comprise protein antigens: Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide from Haemophilus influenzae and an UspA2 polypeptide from Moraxella catarrhalis.

### Protein D

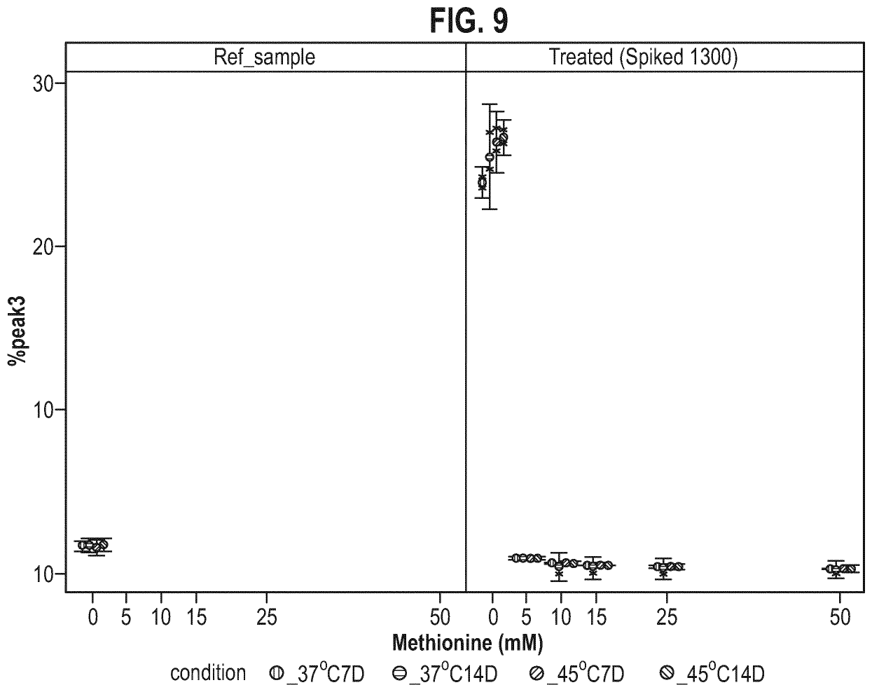

Immunogenic compositions of the invention comprise a Protein D polypeptide. As used herein “Protein D”, “protein D” and “PD” mean Protein D from H. influenzae. Protein D (PD) from Haemophilus influenzae is described in WO91/18926 and EP0594610. Protein D from Haemophilus influenzae may be a Protein D sequence from FIG. 9 (FIGS. 9a and 9b together, 364 amino acids) of EP0594610 (SEQ ID NO: 1). Protein D polypeptides may be full length Protein D or an immunogenic fragment thereof (e.g. Protein D polypeptides are described in WO00/56360). For example, the Protein D polypeptide may comprise (or consist) of the Protein D fragment described in EP0594610 beginning at the sequence SSHSSNMANT (SerSerHisSerSerAsnMetAlaAsnThr) (SEQ ID NO: 3), and lacking the 19 N-terminal amino acids from FIG. 9 of EP0594610, optionally with the addition of the tripeptide MDP from NS1 fused to the N-terminal of said Protein D fragment (348 amino acids) (i.e. SEQ ID NO:2). Thus, in an embodiment, the Protein D polypeptide may comprise (or consist) of the amino acid sequence of SEQ ID NO: 2. In an embodiment, the Protein D polypeptide is not conjugated to a polysaccharide, e.g. a polysaccharide from Streptococcus pneumoniae. In an embodiment, the Protein D polypeptide is not conjugated to a polysaccharide from Streptococcus pneumoniae. In an embodiment, the Protein D polypeptide is a free protein (e.g. unconjugated). In an embodiment, the Protein D polypeptide is unlipidated.

SEQ ID NO 1: Protein D (364 amino acids)
MetLysLeuLysThrLeuAlaLeuSerLeuLeuAlaAlaGlyValLeu
AlaGlyCysSerSerHisSerSerAsnMetAlaAsnThrGlnMetLys
SerAspLysIleIleIleAlaHisArgGlyAlaSerGlyTyrLeuPro
GluHisThrLeuGluSerLysAlaLeuAlaPheAlaGlnGlnAlaAsp
TyrLeuGluGlnAspLeuAlaMetThrLysAspGlyArgLeuValVal
IleHisAspHisPheLeuAspGlyLeuThrAspValAlaLysLysPhe
ProHisArgHisArgLysAspGlyArgTyrTyrValIleAspPheThr
LeuLysGluIleGlnSerLeuGluMetThrGluAsnPheGluThrLys
AspGlyLysGlnAlaGlnValTyrProAsnArgPheProLeuTrpLys
SerHisPheArgIleHisThrPheGluAspGluIleGluPheIleGln
GlyLeuGluLysSerThrGlyLysLysValGlyIleTyrProGluIle
LysAlaProTrpPheHisHisGlnAsnGlyLysAspIleAlaAlaGlu
ThrLeuLysValLeuLysLysTyrGlyTyrAspLysLysThrAspMet
ValTyrLeuGlnThrPheAspPheAsnGluLeuLysArgIleLysThr
GluLeuLeuProGlnMetGlyMetAspLeuLysLeuValGlnLeuIle
AlaTyrThrAspTrpLysGluThrGlnGluLysAspProLysGlyTyr
TrpValAsnTyrAsnTyrAspTrpMetPheLysProGlyAlaMetAla
GluValValLysTyrAlaAspGlyValGlyProGlyTrpTyrMetLeu
ValAsnLysGluGluSerLysProAspAsnIleValTyrThrProLeu
ValLysGluLeuAlaGlnTyrAsnValGluValHisProTyrThrVal
ArgLysAspAlaLeuProGluPhePheThrAspValAsnGlnMetTyr
AspAlaLeuLeuAsnLysSerGlyAlaThrGlyValPheThrAspPhe
ProAspThrGlyValGluPheLeuLysGlyIleLys
SEQ ID NO: 2: Protein D fragment with MDP
tripeptide from NS1 (348 amino acids)
MetAspProSerSerHisSerSerAsnMetAlaAsnThrGlnMetLys
SerAspLysIleIleIleAlaHisArgGlyAlaSerGlyTyrLeuPro
GluHisThrLeuGluSerLysAlaLeuAlaPheAlaGlnGlnAlaAsp
TyrLeuGluGlnAspLeuAlaMetThrLysAspGlyArgLeuValVal
IleHisAspHisPheLeuAspGlyLeuThrAspValAlaLysLysPhe
ProHisArgHisArgLysAspGlyArgTyrTyrValIleAspPheThr
LeuLysGluIleGlnSerLeuGluMetThrGluAsnPheGluThrLys
AspGlyLysGlnAlaGlnValTyrProAsnArgPheProLeuTrpLys
SerHisPheArgIleHisThrPheGluAspGluIleGluPheIleGln
GlyLeuGluLysSerThrGlyLysLysValGlyIleTyrProGluIle
LysAlaProTrpPheHisHisGlnAsnGlyLysAspIleAlaAlaGlu
ThrLeuLysValLeuLysLysTyrGlyTyrAspLysLysThrAspMet
ValTyrLeuGlnThrPheAspPheAsnGluLeuLysArgIleLysThr
GluLeuLeuProGlnMetGlyMetAspLeuLysLeuValGlnLeuIle
AlaTyrThrAspTrpLysGluThrGlnGluLysAspProLysGlyTyr
TrpValAsnTyrAsnTyrAspTrpMetPheLysProGlyAlaMetAla
GluValValLysTyrAlaAspGlyValGlyProGlyTrpTyrMetLeu
ValAsnLysGluGluSerLysProAspAsnIleValTyrThrProLeu
ValLysGluLeuAlaGlnTyrAsnValGluValHisProTyrThrVal
ArgLysAspAlaLeuProGluPhePheThrAspValAsnGlnMetTyr
AspAlaLeuLeuAsnLysSerGlyAlaThrGlyValPheThrAspPhe
ProAspThrGlyValGluPheLeuLysGlyIleLys

Thus the Protein D polypeptide sequence for use in the present invention can be modified, for example by truncation of N-terminal or C-terminal residues (e.g. deletion of the N-terminal 19 amino acid residues), by addition of amino acid residues (e.g. the addition of the tripeptide MDP), or by conservative amino acid substitutions. In an embodiment, the immunogenic composition comprises a Protein D polypeptide having at least 70%, 80%, 85%, 90%, 91%, 92%, 93%, 94%, 95%, 96%, 97%, 98%, 99%, or 100% identity to SEQ ID NO: 1. Immunogenic fragments of Protein D may comprise immunogenic fragments of at least 7, 10, 15, 20, 25, 30 or 50 contiguous amino acids of SEQ ID NO: 1. For example, immunogenic fragments of Protein D may comprise immunogenic fragments of at least 7, 10, 15, 20, 25, 30, 50, 100, 200 or 300 contiguous amino acids of SEQ ID NO: 1, up to 363 contiguous amino acids of SEQ ID NO: 1. The Protein D polypeptide sequence (e.g. SEQ ID NO: 1) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 1. In another embodiment, the immunogenic composition comprises a Protein D polypeptide having at least 70%, 80%, 85%, 90%, 91%, 92%, 93%, 94%, 95%, 96%, 97%, 98%, 99%, or 100% identity to SEQ ID NO: 2. Immunogenic fragments of Protein D may comprise at least 7, 10, 15, 20, 25, 30 or 50 contiguous amino acids of SEQ ID NO: 2. For example, immunogenic fragments of Protein D may comprise immunogenic fragments of at least 7, 10, 15, 20, 25, 30, 50, 100, 200 or 300 contiguous amino acids of SEQ ID NO: 2, up to 347 contiguous amino acids of SEQ ID NO: 2. Immunogenic fragments of Protein D may comprise 100, 200, 300, 310, 320, 330 or 340 contiguous amino acids of SEQ ID NO: 2. The Protein D polypeptide sequence (e.g. SEQ ID NO: 2) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 2.

### Protein E

Immunogenic compositions of the invention comprise Protein E from Haemophilus influenzae or an immunogenic fragment thereof.

Protein E (PE) is an outer membrane lipoprotein with adhesive properties. It plays a role in the adhesion/invasion of non-typeable Haemophilus influenzae (NTHi) to epithelial cells. (J. Immunology 183: 2593-2601 (2009); The Journal of Infectious Diseases 199:522-531 (2009), Microbes and Infection 10:87-96 (2008)). It is highly conserved in both encapsulated Haemophilus influenzae and non-typeable H. influenzae and has a conserved epithelial binding domain (The Journal of Infectious Diseases 201:414-419 (2010)). Thirteen different point mutations have been described in different Haemophilus species when compared with Haemophilus influenzae Rd as a reference strain. Its expression is observed on both logarithmic growing and stationary phase bacteria. (WO2007/084053). Protein E is also involved in human complement resistance through binding vitronectin. (Immunology 183: 2593-2601 (2009)). PE binds vitronectin which is an important inhibitor of the terminal complement pathway. (J. Immunology 183:2593-2601 (2009)).

As used herein “Protein E”, “protein E”, “Prot E”, and “PE” mean Protein E from H. influenzae. Protein E may comprise (or consist) of the amino acid sequence of SEQ ID NO: 4 (corresponding to SEQ ID NO: 4 of WO2012/139225A1): (MKKIILTLSL GLLTACSAQI QKAEQNDVKL APPTDVRSGY IRLVKNVNYY IDSESIWVDN QEPQIVHFDA VVNLDKGLYV YPEPKRYARS VRQYKILNCA NYHLTQVRTD FYDEFWGQGL RAAPKKQKKH TLSLTPDTTL YNAAQIICAN YGEAFSVDKK).

In particular embodiments, the immunogenic composition comprises Protein E from Haemophilus influenzae or an immunogenic fragment thereof, suitably having at least 70%, 80%, 85%, 90%, 91%, 92%, 93%, 94%, 95%, 96%, 97%, 98%, 99%, or 100% identity to SEQ ID NO: 4. In another embodiment, the immunogenic composition comprises an immunogenic fragment of Protein E from Haemophilus influenzae, suitably having at least 70%, 80%, 85%, 90%, 91%, 92%, 93%, 94%, 95%, 96%, 97%, 98%, or 99% identity to SEQ ID NO: 4. For example, immunogenic fragments of Protein E may comprise at least 7, 10, 15, 20, 25, 30 or 50 contiguous amino acids of SEQ ID NO: 4. For example, immunogenic fragments of Protein E may comprise at least 7, 10, 15, 20, 25, 30, 50, 100 or 150 contiguous amino acids of SEQ ID NO: 4, up to 159 contiguous amino acids of SEQ ID NO: 4. The immunogenic fragments may elicit antibodies which can bind SEQ ID NO: 4.

In another embodiment, the immunogenic composition comprises Protein E from Haemophilus influenzae or an immunogenic fragment thereof having at least 70%, 80%, 85%, 90%, 91%, 92%, 93%, 94%, 95%, 96%, 97%, 98%, 99%, or 100% identity to SEQ ID NO: 5 (corresponding to SEQ ID NO: 125 of WO2012/139225A1):

SEQ ID NO: 5: Amino acids 20-160 of Protein E
I QKAEQNDVKL APPTDVRSGY IRLVKNVNYY IDSESIWWDN
QEPQIVHFDA VVNLDKGLYV YPEPKRYARS VRQYKILNCA
NYHLTQVRTD FYDEFWGQGL RAAPKKQKKH TLSLTPDTTL
YNAAQIICAN YGEAFSVDKK

In another embodiment, the immunogenic composition comprises an immunogenic fragment of Protein E from Haemophilus influenzae, suitably having at least 70%, 80%, 85%, 90%, 91%, 92%, 93%, 94%, 95%, 96%, 97%, 98%, 99%, or 100% identity to SEQ ID NO: 5 (corresponding to SEQ ID NO: 125 of WO2012/139225A1). In another embodiment, the immunogenic composition comprises an immunogenic fragment of Protein E from Haemophilus influenzae, comprising (or consisting) of the amino acid sequence of SEQ ID NO: 5 (corresponding to SEQ ID NO: 125 of WO2012/139225A1).

### PilA

Immunogenic compositions of the invention comprise PilA from Haemophilus influenzae or an immunogenic fragment thereof.

Pilin A (PilA) is likely the major pilin subunit of H. influenzae Type IV Pilus (Tfp) involved in twitching motility (Infection and Immunity, 73: 1635-1643 (2005)). NTHi PilA is a conserved adhesin expressed in vivo. It has been shown to be involved in NTHi adherence, colonization and biofilm formation. (Molecular Microbiology 65: 1288-1299 (2007)).

As used herein “PilA” means Pilin A from H. influenzae. PilA may comprise (or consist) the protein sequence of SEQ ID NO: 6 (corresponding to SEQ ID NO: 58 of WO2012/139225A1)

(MKLTTQQTLK KGFTLIELMI VIAIIAILAT IAIPSYQNYT
KKAAVSELLQ ASAPYKADVE LCVYSTNETT NCTGGKNGIA
ADITTAKGYV KSVTTSNGAI TVKGDGTLAN MEYILQATGN
AATGVTWTTT CKGTDASLFP ANFCGSVTQ).

In particular embodiments, the immunogenic composition comprises PilA from Haemophilus influenzae or an immunogenic fragment thereof, suitably having at least 70%, 80%, 85%, 90%, 91%, 92%, 93%, 94%, 95%, 96%, 97%, 98%, 99%, or 100% identity to SEQ ID NO: 6. In another embodiment, the immunogenic composition comprises an immunogenic fragment of PilA from Haemophilus influenzae, suitably having at least 70%, 80%, 85%, 90%, 91%, 92%, 93%, 94%, 95%, 96%, 97%, 98%, or 99% identity to SEQ ID NO: 6. For example, immunogenic fragments of PilA may comprise at least 7, 10, 15, 20, 25, 30 or 50 contiguous amino acids of SEQ ID NO: 6. For example, immunogenic fragments of PilA may comprise at least 7, 10, 15, 20, 25, 30, 50 or 100 contiguous amino acids of SEQ ID NO: 6, up to 148 contiguous amino acids of SEQ ID NO: 6. The immunogenic fragments may elicit antibodies which can bind SEQ ID NO: 6.

In another embodiment, the immunogenic composition comprises PilA from Haemophilus influenzae or an immunogenic fragment thereof having at least 70%, 80%, 85%, 90%, 91%, 92%, 93%, 94%, 95%, 96%, 97%, 98%, 99%, or 100% identity to SEQ ID NO: 7 (corresponding to SEQ ID NO: 127 of WO2012/139225A1):

SEQ ID NO: 7 Amino acids 40-149 of PilA from H. influenzae strain 86-028NP
T KKAAVSELLQ ASAPYKADVE LCVYSTNETT NCTGGKNGIA ADITTAKGYV KSVTTSNGAI
TVKGDGTLAN MEYILQATGN AATGVTWTTT CKGTDASLFP ANFCGSVTQ.

In another embodiment, the immunogenic composition comprises an immunogenic fragment of PilA from Haemophilus influenzae, suitably having at least 70%, 80%, 85%, 90%, 91%, 92%, 93%, 94%, 95%, 96%, 97%, 98%, 99%, or 100% identity to SEQ ID NO: 7 (corresponding to SEQ ID NO: 127 of WO2012/139225A1). In another embodiment, the immunogenic composition comprises an immunogenic fragment of PilA from Haemophilus influenzae, comprising (or consisting) of the amino acid sequence of SEQ ID NO: 7 (corresponding to SEQ ID NO: 127 of WO2012/139225A1).

### PE-PilA Fusion Protein

Protein E from Haemophilus influenzae or an immunogenic fragment thereof and PilA from Haemophilus influenzae or an immunogenic fragment thereof may be presented as a fusion protein. Thus, in an embodiment, the immunogenic composition comprises Protein E from Haemophilus influenzae or an immunogenic fragment thereof and PilA from Haemophilus influenzae or an immunogenic fragment thereof presented as a fusion protein. Suitably, the fusion protein may comprise Protein E from Haemophilus influenzae or an immunogenic fragment thereof at the N-terminus and PilA from Haemophilus influenzae or an immunogenic fragment thereof at the C-terminus of the fusion protein (a PE-PilA fusion protein). In another embodiment, the immunogenic composition comprises protein E from Haemophilus influenzae or an immunogenic fragment thereof and PilA from Haemophilus influenzae or an immunogenic fragment thereof as a PE-PilA fusion protein having at least 70%, 80%, 85%, 90%, 91%, 92%, 93%, 94%, 95%, 96%, 97%, 98%, 99%, or 100% identity to SEQ ID NO: 8 (LVL-735, corresponding to SEQ ID NO: 194 of WO2012/139225A1). In particular, the immunogenic composition comprises an immunogenic fragment of Protein E from Haemophilus influenzae and an immunogenic fragment of PilA from Haemophilus influenzae as a PE-PilA fusion protein having at least 70%, 80%, 85%, 90%, 91%, 92%, 93%, 94%, 95%, 96%, 97%, 98%, 99%, or 100% identity to SEQ ID NO: 8 (LVL-735, corresponding to SEQ ID NO: 194 of WO2012/139225A1).

SEQ ID NO: 8: LVL735 (protein): (pelB sp)(ProtE aa 20-160)(GG)(PilA aa40-149):
MKYLLPTAAA GLLLLAAQPA MAIQKAEQND VKLAPPTDVR SGYIRLVKNV NYYIDSESIW
VDNQEPQIVH FDAVVNLDKG LYVYPEPKRY ARSVRQYKIL NCANYHLTQV RTDFYDEFWG
QGLRAAPKKQ KKHTLSLTPD TTLYNAAQII CANYGEAFSV DKKGGTKKAA VSELLQASAP
YKADVELCVY STNETTNCTG GKNGIAADIT TAKGYVKSVT TSNGAITVKG DGTLANMEYI
LQATGNAATG VTWTTTCKGT DASLFPANFC GSVTQ

In an embodiment, the immunogenic composition comprises an immunogenic fragment of Protein E from Haemophilus influenzae and an immunogenic fragment of PilA from Haemophilus influenzae as a PE-PilA fusion protein comprising (or consisting) of the amino acid sequence of SEQ ID NO: 8 (LVL-735 corresponding to SEQ ID NO: 194 of WO2012/139225A1).

In another embodiment, the immunogenic composition comprises Protein E from Haemophilus influenzae or an immunogenic fragment thereof and PilA from Haemophilus influenzae or an immunogenic fragment thereof as a fusion protein (e.g. PE-PilA fusion protein) having at least 70%, 80%, 85%, 90%, 91%, 92%, 93%, 94%, 95%, 96%, 97%, 98%, 99%, or 100% identity to SEQ ID NO: 9 (LVL-735 wherein the signal peptide has been removed, corresponding to SEQ ID NO: 219 of WO2012/139225A1). In particular, the immunogenic composition comprises an immunogenic fragment of Protein E from Haemophilus influenzae and an immunogenic fragment of PilA from Haemophilus influenzae as a PE-PilA fusion protein having at least 70%, 80%, 85%, 90%, 91%, 92%, 93%, 94%, 95%, 96%, 97%, 98%, 99%, or 100% identity to SEQ ID NO: 9 (LVL-735 wherein the signal peptide has been removed, corresponding to SEQ ID NO: 219 of WO2012/139225A1).

SEQ ID NO: 9: PE-PilA fusion protein without signal peptide
IQKAEQND VKLAPPTDVR SGYIRLVKNV NYYIDSESIW VDNQEPQIVH FDAVVNLDKG
LYVYPEPKRY ARSVRQYKIL NCANYHLTQV RTDFYDEFWG QGLRAAPKKQ KKHTLSLTPD
TTLYNAAQII CANYGEAFSV DKKGGTKKAA VSELLQASAP YKADVELCVY STNETTNCTG
GKNGIAADIT TAKGYVKSVT TSNGAITVKG DGTLANMEYI LQATGNAATG VTWTTTCKGT
DASLFPANFC GSVTQ

In an embodiment, the immunogenic composition comprises an immunogenic fragment of Protein E from Haemophilus influenzae and an immunogenic fragment of PilA from Haemophilus influenzae as a PE-PilA fusion protein comprising (or consisting) of the amino acid sequence of SEQ ID NO: 9 (LVL-735 wherein the signal peptide has been removed, corresponding to SEQ ID NO: 219 of WO2012/139225A1).

The immunogenicity of immunogenic fragments of Protein E (PE) and Pilin A (PilA) may be measured as described in WO2012/139225A1.

### UspA2

Immunogenic compositions of the present invention comprise an UspA2 polypeptide.

Ubiquitous surface protein A2 (UspA2) is a trimeric autotransporter that appears as a lollipop-shared structure in electron micrographs (Hoiczyk et al. EMBO J. 19: 5989-5999 (2000)). It is composed of a N-terminal head, followed by a stalk which ends by an amphipathic helix and a C-terminal membrane domain. (Hoiczyk et al. EMBO J. 19: 5989-5999 (2000)). UspA2 contains a very well conserved domain (Aebi et al., Infection & Immunity 65(11) 4367-4377 (1997)), which is recognized by a monoclonal antibody that was shown protective upon passive transfer in a mouse Moraxella catarrhalis challenge model (Helminnen et al. J Infect Dis. 170(4): 867-72 (1994)). UspA2 has been shown to interact with host structures and extracellular matrix proteins like fibronectin (Tan et al., J Infect Dis. 192(6): 1029-38 (2005)) and laminin (Tan et al., J Infect Dis. 194(4): 493-7 (2006)), suggesting it can play a role at an early stage of Moraxella catarrhalis infection. UspA2 also seems to be involved in the ability of Moraxella catarrhalis to resist the bactericidal activity of normal human serum. (Attia A S et al. Infect Immun 73(4): 2400-2410 (2005)). It (i) binds the complement inhibitor C4 bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin. (de Vries et al., Microbiol Mol Biol Rev. 73(3): 389-406 (2009)).

As used herein “UspA2” means Ubiquitous surface protein A2 from Moraxella catarrhalis. UspA2 may comprise (or consist) of the amino acid sequence of SEQ ID NO: 10 from ATCC 25238 (corresponding to SEQ ID NO: 1 of WO2015/125118A1):

(SEQ ID NO: 10)
MKTMKLLPLKIAVTSAMIIGLGAASTANAQAKNDITLEDLPYLIKKIDQ
NELEADIGDITALEKYLALSQYGNILALEELNKALEELDEDVGWNQNDI
ANLEDDVETLTKNQNALAEQGEAIKEDLQGLADFVEGQEGKILQNETSI
KKNTQRNLVNGFEIEKNKDAIAKNNESIEDLYDFGHEVAESIGEIHAHN
EAQNETLKGLITNSIENTNNITKNKADIQALENNVVEELFNLSGRLIDQ
KADIDNNINNIYELAQQQDQHSSDIKTLKKNVEEGLLELSGHLIDQKTD
IAQNQANIQDLATYNELQDQYAQKQTEAIDALNKASSENTQNIEDLAAY
NELQDAYAKQQTEAIDALNKASSENTQNIEDLAAYNELQDAYAKQQTEA
IDALNKASSENTQNIAKNQADIANNINNIYELAQQQDQHSSDIKTLAKA
SAANTDRIAKNKADADASFETLTKNQNTLIEKDKEHDKLITANKTAIDA
NKASADTKFAATADAITKNGNAITKNAKSITDLGTKVDGFDSRVTALDT
KVNAFDGRITALDSKVENGMAAQAALSGLFQPYSVGKFNATAALGGYGS
KSAVAIGAGYRVNPNLAFKAGAAINTSGNKKGSYNIGVNYEF

as well as sequences having at least or exactly 63%, 66%, 70%, 72%, 74%, 75%, 77%, 80%, 84%, 85%, 90%, 95%, 96%, 97%, 98%, 99% or 100% identity, over the entire length, to SEQ ID NO: 10.

UspA2 polypeptides may be full length UspA2 or an immunogenic fragment thereof. In particular embodiments, the immunogenic composition comprises an UspA2 polypeptide having at least 70%, 80%, 85%, 90%, 91%, 92%, 93%, 94%, 95%, 96%, 97%, 98%, 99%, or 100% identity to SEQ ID NO: 10. In another embodiment, the immunogenic composition comprises an immunogenic fragment of UspA2 from Moraxella catarrhalis having at least 70%, 80%, 85%, 90%, 91%, 92%, 93%, 94%, 95%, 96%, 97%, 98%, or 99% identity to SEQ ID NO: 10. For example, immunogenic fragments of UspA2 may comprise at least 7, 10, 15, 20, 25, 30 or 50 contiguous amino acids of SEQ ID NO: 10. For example, immunogenic fragments of UspA2 may comprise at least 7, 10, 15, 20, 25, 30, 50, 100, 200, 300, 400, 500 or 600 contiguous amino acids of SEQ ID NO: 10, up to 629 contiguous amino acids of SEQ ID NO: 10. The immunogenic fragments may elicit antibodies which can bind SEQ ID NO: 10.

UspA2 as described in SEQ ID NO: 10 contains a signal peptide (for example, amino acids 1 to 29 of SEQ ID NO: 10), a laminin binding domain (for example, amino acids 30 to 177 of SEQ ID NO: 10), a fibronectin binding domain (for example, amino acids 165 to 318 of SEQ ID NO: 10) (Tan et al. JID 192: 1029-38 (2005)), a C3 binding domain (for example, amino acids 30 to 539 of SEQ ID NO: 10 (WO2007/018463), or a fragment of amino acids 30 to 539 of SEQ ID NO: 10, for example, amino acids 165 to 318 of SEQ ID NO: 1 (Hallstrom T et al. J. Immunol. 186: 3120-3129 (2011)), an amphipathic helix (for example, amino acids 519 to 564 of SEQ ID NO: 10 or amino acids 520-559 of SEQ ID NO:10, identified using different prediction methods) and a C terminal anchor domain (for example, amino acids 576 to 630 amino acids of SEQ ID NO: 10 (Brooks et al., Infection & Immunity, 76(11), 5330-5340 (2008)). In an embodiment, an UspA2 polypeptide contains a laminin binding domain and a fibronectin binding domain. In an additional embodiment, an immunogenic fragment of UspA2 contains a laminin binding domain, a fibronectin binding domain and a C3 binding domain. In a further embodiment, an UspA2 polypeptide, contains a laminin binding domain, a fibronectin binding domain, a C3 binding domain and an amphipathic helix.

UspA2 amino acid differences have been described for various Moraxella catarrhalis species. See for example, J Bacteriology 181(13):4026-34 (1999), Infection and Immunity 76(11):5330-40 (2008) and PLoS One 7(9):e45452 (2012). An UspA2 polypeptide, may comprise (or consist) of an amino acid sequence that differs from SEQ ID NO: 10 at any one or more amino acid selected from the group consisting of: AA (amino acid) 30 to 298, AA 299 to 302, AA 303 to 333, AA 334 to 339, AA 349, AA 352 to 354, AA 368 to 403, AA 441, AA 451 to 471, AA 472, AA474 to 483, AA 487, AA 490, AA 493, AA 529, AA 532 or AA 543. An UspA2 polypeptide, may comprise (or consist) of an amino acid sequence that differs from SEQ ID NO: 10 in that it contains an amino acid insertion in comparison to SEQ ID NO: 10. UspA2 may comprise (or consist) of an amino acid sequence that differs from SEQ ID NO: 10 at any one of the amino acid differences in SEQ ID NO: 22 through SEQ ID NO: 58. For example, SEQ ID NO: 10 may contain K instead of Q at amino acid 70, Q instead of G at amino acid 135 and/or D instead of N at amino acid 216.

UspA2 may be UspA2 from M. catarrhalis strain ATCC (a US registered trademark) 25238™, American 2933. American 2912, American 2908, Finnish 307, Finnish 353, Finnish 358, Finnish 216, Dutch H2, Dutch F10, Norwegian 1, Norwegian 13, Norwegian 20, Norwegian 25, Norwegian 27, Norwegian 36, BC5SV, Norwegian 14, Norwegian 3, Finish 414, Japanese Z7476, Belgium Z7530, German Z8063, American 012E, Greek MC317, American V1122, American P44, American V1171, American TTA24, American 035E, American SP12-6, American SP12-5, Swedish BC5, American 7169, Finnish FIN2344, American V1118, American V1145 or American V1156. UspA2 may be UspA2 as set forth in any of SEQ ID NO: 10 or SEQ ID NO: 22-SEQ ID NO: 38. UspA2 may be UspA2 from another source which corresponds to the sequence of UspA2 in any one of SEQ ID NO: 10 or SEQ ID NO: 22-SEQ ID NO: 58. Corresponding UspA2 sequences may be determined by one skilled in the art using various algorithms. For example, the Gap program or the Needle program may be used to determine UspA2 sequences corresponding to any one of SEQ ID NO: 10 or SEQ ID NO: 22-SEQ ID NO: 58.

UspA2 may be a sequence having at least 95% identity, over the entire length, to any of SEQ ID NO: 10 or SEQ ID NO: 22-SEQ ID NO: 58. In particular embodiments, UspA2 may be a sequence as set forth in an amino acid sequence selected from the group consisting of SEQ ID NO: 10, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58 or any subset of SEQ ID NO: 1 or SEQ ID NO:22 through SEQ ID NO:58.

Immunogenic fragments of UspA2 comprise immunogenic fragments of at least 450 contiguous amino acids of SEQ ID NO: 10, 490 contiguous amino acids of SEQ ID NO: 10 (for example, the UspA2 fragment of MC-004 or MC-005), 511 contiguous amino acids of SEQ ID NO: 10 (for example, the UspA2 fragment of construct MC-001, MC-002, MC-003 or MC-004), 534 contiguous amino acids of SEQ ID NO: 10 (for example, the UspA2 fragment of MC-009 or MC-011) or 535 contiguous amino acids of SEQ ID NO: 10 (for example, the UspA2 fragment of MC-007, MC-008 or MC-010). The immunogenic fragments may elicit antibodies which can bind SEQ ID NO: 10.

Immunogenic fragments of UspA2 may comprise immunogenic fragments of at least 450, 490, 511, 534 or 535 contiguous amino acids of SEQ ID NO: 10. For example, immunogenic fragments of UspA2 may comprise immunogenic fragments of at least 450, 490, 511, 534 or 535 contiguous amino acids of SEQ ID NO: 10 up to 629 amino acids of SEQ ID NO: 10. Immunogenic fragments of UspA2 may comprise immunogenic fragments of UspA2, for example any of the UspA2 constructs MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21). The immunogenic fragments may elicit antibodies which can bind the full length sequence from which the fragment is derived.

In another embodiment, the immunogenic composition comprises an UspA2 polypeptide having at least 70%, 80%, 85%, 90%, 91%, 92%, 93%, 94%, 95%, 96%, 97%, 98%, 99%, or 100% identity to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21). For example, the immunogenic composition may comprise an UspA2 polypeptide having at least 70%, 80%, 85%, 90%, 91%, 92%, 93%, 94%, 95%, 96%, 97%, 98%, 99%, or 100% identity to MC009 SEQ ID NO: 19 (corresponding to SEQ ID NO: 69 of WO2015/125118A1).

SEQ ID NO: 19 MC-009 (Protein)-(M)(UspA2 31-564)(HH)
MAKNDITLEDLPYLIKKIDQNELEADIGDITALEKYLALSQYGNILALEELNKALEELDEDVGWN
QNDIANLEDDVETLTKNQNALAEQGEAIKEDLQGLADFVEGQEGKILQNETSIKKNTQRNLVNGFEIEK
NKDAIAKNNESIEDLYDFGHEVAESIGEIHAHNEAQNETLKGLITNSIENTNNITKNKADIQALENNVVEE
LFNLSGRLIDQKADIDNNINNIYELAQQQDQHSSDIKTLKKNVEEGLLELSGHLIDQKTDIAQNQANIQD
LATYNELQDQYAQKQTEAIDALNKASSENTQNIEDLAAYNELQDAYAKQQTEAIDALNKASSENTQNIE
DLAAYNELQDAYAKQQTEAIDALNKASSENTQNIAKNQADIANNINNIYELAQQQDQHSSDIKTLAKAS
AANTDRIAKNKADADASFETLTKNQNTLIEKDKEHDKLITANKTAIDANKASADTKFAATADAITKNGNAI
TKNAKSITDLGTKVDGFDSRVTALDTKVNAFDGRITALDSKVENGMAAQAAHH

In an embodiment, the immunogenic composition may comprise an UspA2 polypeptide comprising (or consisting) of an amino acid sequence of SEQ ID NO: 19 (corresponding to SEQ ID NO: 69 of WO2015/125118A1).

Immunogenicity of UspA2 polypeptides may be measured as described in WO2015/125118A1.

### Adjuvants

An immunogenic composition of the invention may further comprise a pharmaceutically acceptable adjuvant.

Suitable adjuvants include an aluminum salt such as aluminum hydroxide gel or aluminum phosphate or alum, but may also be a salt of calcium, magnesium, iron or zinc, or may be an insoluble suspension of acylated tyrosine, or acylated sugars, cationically or anionically derivatized saccharides, or polyphosphazenes. In particular embodiments, the protein antigen may be adsorbed onto aluminium phosphate. In another embodiment, the protein antigen may be adsorbed onto aluminium hydroxide. In a third embodiment, alum may be used as an adjuvant.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see GB 2220211 A); and a combination of monophosphoryl lipid A, e.g. 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (Thoelen et al. Vaccine (1998) 16:708-14; EP 689454-B1).

In an embodiment, the pharmaceutically acceptable adjuvant is AS01. AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In particular embodiments, the immunogenic composition or vaccine comprises AS01. In another embodiment, the immunogenic composition or vaccine comprises AS01B or AS01E. In a particular embodiment, the immunogenic composition or vaccine comprises AS01E.

In further embodiments, the pharmaceutically acceptable adjuvant may be AS02, AS03 or AS04. AS02 is an Adjuvant system containing MPL and QS21 in an oil/water emulsion. AS02V is an Adjuvant System containing MPL and QS21 in an oil/water emulsion (50 μg MPL and 50 μg QS21). AS03 is an Adjuvant System containing α-Tocopherol and squalene in an oil/water (o/w) emulsion. AS03_{A} is an Adjuvant System containing α-Tocopherol and squalene in an o/w emulsion (11.86 mg tocopherol). AS03_{B} is an Adjuvant System containing α-Tocopherol and squalene in an o/w emulsion (5.93 mg tocopherol). AS03_{C} is an Adjuvant System containing α-Tocopherol and squalene in an o/w emulsion (2.97 mg tocopherol). In particular embodiments, the immunogenic composition or vaccine comprises AS03. AS04 is an Adjuvant System containing MPL (50 μg MPL) adsorbed on an aluminum salt (500 μg Al³⁺). In particular embodiments, the immunogenic composition or vaccine comprises AS04.

A system involving the use of QS21 and 3D-MPL is disclosed in WO 94/00153. A composition wherein the QS21 is quenched with cholesterol is disclosed in WO 96/33739. An additional adjuvant formulation involving QS21, 3D-MPL and tocopherol in an oil in water emulsion is described in WO 95/17210. In particular embodiments the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO 0226757 and WO 03507822) are also preferential inducers of a TH1 response and are suitable for use in the present invention.

Additional adjuvants are those selected from the group of metal salts, oil in water emulsions, Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

### Dosage

The present invention provides immunogenic compositions comprising 15 to 30 μg/ml PE-PilA fusion protein (optionally a PE-PilA fusion protein of SEQ ID NO: 9), 15 to 30 μg/ml Protein D polypeptide (optionally a Protein D polypeptide of SEQ ID NO: 2) and 6 to 9 μg/ml UspA2 polypeptide (optionally an UspA2 polypeptide of SEQ ID NO: 19). The present invention also provides immunogenic compositions comprising 20 to 25 μg/ml PE-PilA fusion protein (optionally a PE-PilA fusion protein of SEQ ID NO: 9), 20 to 25 μg/ml Protein D polypeptide (optionally a Protein D polypeptide of SEQ ID NO: 2) and 6 to 9 μg/ml UspA2 polypeptide (optionally an UspA2 polypeptide of SEQ ID NO: 19). The present invention also provides immunogenic compositions comprising 9 to 15 μg (e.g. 9 to 13 μg) PE-PilA fusion protein (optionally a PE-PilA fusion protein of SEQ ID NO: 9), 9 to 15 μg (e.g. 9 to 13 μg) Protein D polypeptide (optionally a Protein D polypeptide of SEQ ID NO: 2), 3 to 5 μg UspA2 polypeptide (optionally an UspA2 polypeptide of SEQ ID NO: 19). In an embodiment an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19). In an embodiment, an immunogenic composition of the invention comprises 9 to 15 μg PE-PilA fusion protein (e.g. SEQ ID NO: 9), 9 to 15 μg Protein D polypeptide (e.g. SEQ ID NO: 2), 3 to 5 μg UspA2 polypeptide (e.g. SEQ ID NO: 19) in a solid dosage (e.g. freeze-dried) form. In an embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19) in a solid dosage (e.g. freeze-dried) form.

The dose may be administered to the subject, e.g. human, as a single unit dose. Several separate unit doses may also be administered. For example, separate unit doses may be administered as separate priming doses within the first year of life or as separate booster doses given at regular intervals (for example, every 1, 5 or 10 years). In an embodiment, the present invention provides an immunogenic composition in a unit dose form. Immunogenic compositions of the invention may be administered to patients in unit doses, ranging between 0.1 to 1 ml, e.g. 0.5 ml. References to 0.5 ml will be understood to include normal variance e.g. 0.5 ml+/−0.05 ml. Thus, the present invention also provides an immunogenic composition comprising 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19) in a 0.5 ml dose.

The amount of protein antigen in an immunogenic composition which is required to achieve the desired therapeutic or biological effect will depend on a number of factors such as means of administration, the recipient and the type and severity of the condition being treated, and will be ultimately at the discretion of the attendant physician or veterinarian. For example, two specific immunogenic compositions were evaluated in a mouse Moraxella catarrhalis lung inflammation model in WO2015125118 (see Example 14 of WO2015125118):
PD 10 μg/PE-PilA (LVL735 construct, as described in WO2012/139225) 10 μg/UspA2 (MC009 construct, as described in WO2015125118) 10 μg/AS01E
OR
PD 10 μg/PE-PilA (LVL735 construct, as described in WO2012/139225) 10 μg/UspA2 (MC009 construct, as described in WO2015125118) 3.3 μg/AS01E

Thus, in particular embodiments the immunogenic composition of the invention comprises 10 μg PE-PilA fusion protein (e.g. SEQ ID NO: 9), 10 μg Protein D polypeptide (e.g. SEQ ID NO: 2) and 10 μg UspA2 polypeptide (e.g. SEQ ID NO: 19), suitably in a 0.5 ml dose. In another embodiment the immunogenic composition of the invention comprises 10 μg PE-PilA fusion protein (e.g. SEQ ID NO: 9), 10 μg Protein D polypeptide (e.g. SEQ ID NO: 2) and 3.3 μg UspA2 polypeptide (e.g. SEQ ID NO: 19), suitably in a 0.5 ml dose. In another embodiment the immunogenic composition of the invention comprises 20 μg/ml PE-PilA fusion protein (e.g. SEQ ID NO: 9), 20 μg/ml Protein D polypeptide (e.g. SEQ ID NO: 2), 20 μg/ml UspA2 polypeptide (e.g. SEQ ID NO: 19). In another embodiment the immunogenic composition of the invention comprises 20 μg/ml PE-PilA fusion protein (e.g. SEQ ID NO: 9), 20 μg/ml Protein D polypeptide (e.g. SEQ ID NO: 2), 6.6 μg/ml UspA2 polypeptide (e.g. SEQ ID NO: 19).

### Formulation

The immunogenic compositions of the invention may be formulated in liquid form (i.e. solutions or suspensions), or in a solid (e.g. lyophilized/freeze-dried) form. In an embodiment, the immunogenic composition of the invention is in liquid form, suitably in aqueous liquid form. Immunogenic compositions of the invention may be in a liquid form (i) during manufacture of the formulation prior to freeze-drying, and/or (ii) following reconstitution prior to administration to a patient. In another embodiment, the immunogenic composition of the invention is in solid form (e.g. freeze-dried).

In particular embodiments, an immunogenic composition of the invention comprises a PE-PilA fusion protein (e.g. SEQ ID NO: 9), a Protein D polypeptide (e.g. SEQ ID NO: 2), and an UspA2 polypeptide (e.g. SEQ ID NO: 19) in a liquid form. In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), optionally in liquid form. In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), optionally in liquid form. In another embodiment, the present invention provides an immunogenic composition comprising 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19), optionally in a liquid form. Such immunogenic compositions may be prepared during manufacture, e.g. as a bulk immunogenic composition. The bulk immunogenic composition may subsequently be freeze-dried. For example an amount (e.g. 0.5 ml) may be taken and freeze dried, to produce an immunogenic composition of the invention in freeze-dried form. When the immunogenic composition of the invention is in a solid form (e.g. freeze dried) the different amounts of the antigens and excipients (e.g. antioxidant, poloxamer etc.) may be expressed by reference to the initial liquid composition (the bulk immunogenic composition), before the drying step.

The immunogenic composition of the invention may be in solid form (optionally freeze-dried). For the first time, the present invention provides a freeze-dried composition comprising protein antigens: Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide and an UspA2 polypeptide. In particular, a freeze-dried composition is provided which (i) reduces aggregation of protein antigens (in particular aggregation caused by shear stress) and/or (ii) reduces oxidation of protein antigens (in particular oxidation of methionine residues) and thus helps to maintain the structure and function of the protein antigens. Thus, in particular embodiments, the immunogenic composition of the invention comprises a PE-PilA fusion protein (e.g. SEQ ID NO: 9), a Protein D polypeptide (e.g. SEQ ID NO: 2), and an UspA2 polypeptide (e.g. SEQ ID NO: 19) in a solid (e.g. freeze-dried) form. In another embodiment, the immunogenic composition of the invention comprises 9 to 13 μg PE-PilA fusion protein (e.g. SEQ ID NO: 9), 9 to 13 μg Protein D polypeptide (e.g. SEQ ID NO: 2), 3 to 5 μg UspA2 polypeptide (e.g. SEQ ID NO: 19) in a solid dosage (e.g. freeze-dried) form. In another embodiment, the immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19) in a solid dosage (e.g. freeze-dried) form. In another embodiment, the immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19) in a solid dosage (e.g. freeze-dried) form.

Immunogenic compositions of the invention in solid form (e.g. freeze-dried) may be reconstituted prior to vaccine administration. The immunogenic composition in solid (e.g. freeze-dried) form may be reconstituted with water for injection (WFI) and/or an adjuvant (e.g. AS01E) prior to administration. The immunogenic compositions of the invention may further comprise an adjuvant, e.g. AS01E. Thus the immunogenic composition of the invention may be in a liquid form (optionally reconstituted with an aqueous solution comprising an adjuvant e.g. AS01E). In an embodiment, the immunogenic composition comprises a PE-PilA fusion protein (e.g. SEQ ID NO: 9), a Protein D polypeptide (e.g. SEQ ID NO: 2), an UspA2 polypeptide (e.g. SEQ ID NO: 19) and an adjuvant (e.g. AS01E). In another embodiment, the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19) and an adjuvant (e.g. AS01E). In another embodiment, the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19) and an adjuvant (e.g. AS01E). In another embodiment, the immunogenic composition comprises 20 μg/ml PE-PilA fusion protein (e.g. SEQ ID NO: 9), 20 μg/ml Protein D polypeptide (e.g. SEQ ID NO: 2), 6.6 μg/ml UspA2 polypeptide (e.g. SEQ ID NO: 19) and an adjuvant (e.g. AS01E). In another embodiment, the immunogenic composition comprises 10 μg PE-PilA fusion protein (e.g. SEQ ID NO: 9), 10 μg Protein D polypeptide (e.g. SEQ ID NO: 2), 3.3 μg UspA2 polypeptide (e.g. SEQ ID NO: 19) and an adjuvant (e.g. AS01E) in a 0.5 ml dose.

### Poloxamer

The present invention is based, in part, on the identification of the need for and use of a poloxamer in an immunogenic composition comprising Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide, which has been found according to the present invention to mitigate aggregation and non specific adsorption of protein antigens and provide superior properties over other surfactants such as polysorbates. Such aggregation could occur during processing, whenever the proteins are in contact with gas or solid surfaces there is an increased risk of defolding of the protein.

Protein aggregation may be caused by physiochemical stresses, including heat, pressure, pH, agitation, shear forces, freeze-thawing, dehydration, heavy metals, phenolic compounds, silicon oil, denaturants and the like. As described herein in the Examples, it was found that PE-PilA fusion protein and UspA2 polypeptide are susceptible to shear stress that may occur during formulation processes and the addition of poloxamer to the immunogenic composition can mitigate formation of aggregates of a PE-PilA fusion protein and UspA2 polypeptide due to shear stress. It was not previously known that PE-PilA fusion protein and UspA2 polypeptide were susceptible to aggregation due to shear stress and therefore surprisingly the addition of poloxamer provides an improved immunogenic composition (see Example 4 herein). The present invention thus provides immunogenic compositions with improved stability. The present invention provides immunogenic compositions with improved stability compared to immunogenic compositions formulated without poloxamer. According to the present invention, it has also been found that poloxamer may also help reduce aspecific adsorption in immunogenic compositions of the present invention. Thus, the present invention provides improved immunogenic compositions.

Poloxamers are nonionic triblock linear copolymers composed of a central hydrophobic chain of polyoxypropylene (poly(propylene oxide)) flanked by two hydrophilic chains of polyoxyethylene (poly(ethylene oxide). The length of the polymer can vary. The poloxamer may have a molecular weight in the range of 7,500 to 15,000 or 7,500 to 10,000. Suitably, the poloxamer is selected from the group consisting of poloxamer 124, poloxamer 188, poloxamer 237, poloxamer 338 and poloxamer 407. In an embodiment, the poloxomer is poloxamer 188 (PX188).

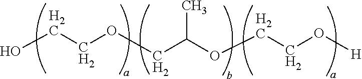

Poloxamer 188 has a molecular weight ranging from 7680 to 9510 Da. Khan et al. (European Journal of Pharmaceutics and Biopharmaceutics, 97 (2015) 60-67) describes generally the use of non-ionic surfactants in therapeutic formulations.

In immunogenic compositions of the present invention, poloxamer (e.g. poloxamer 188) may be present in an amount at least 0.02% (w/v, i.e. weight/volume of the formulation). In immunogenic compositions of the present invention, poloxamer (optionally poloxamer 188) may be present in an amount 0.02 to 0.15% (w/v), suitably 0.03 to 0.15%, 0.03 to 0.09%, 0.04 to 0.15%, 0.04 to 0.1%, 0.04 to 0.09%, 0.04 to 0.06% or 0.04 to 0.05% (w/v). Specifically, the poloxamer may be present in an amount 0.03%. 0.04%, 0.05%, or 0.06% (w/v).

Thus, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19) and poloxamer (e.g. poloxamer 188). In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19) and 0.02 to 0.15% poloxamer (e.g. poloxamer 188) (w/v, i.e. weight/volume of the formulation), optionally in liquid form. In another embodiment, the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19) and 0.02 to 0.15% poloxamer (e.g. poloxamer 188) (w/v, i.e. weight/volume of the formulation), optionally in a liquid form. In an embodiment, the immunogenic composition comprises 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19) and 0.02 to 0.15% poloxamer (e.g. poloxamer 188) (w/v, i.e. weight/volume of the formulation), optionally in a liquid form. Poloxamer may be present in an amount 0.02 to 0.15% (w/v), suitably 0.03 to 0.15%, 0.03 to 0.09%, 0.04 to 0.15%, 0.04 to 0.1%, 0.04 to 0.09%, or 0.04 to 0.06% (w/v). Specifically, the poloxamer may be present in an amount 0.03%. 0.04%, 0.05%, 0.06% or 0.07% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19) and poloxamer (e.g. poloxamer 188) in solid, e.g. freeze-dried form. In another embodiment, an immunogenic composition of the invention comprises 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19) and poloxamer in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19) and poloxamer in a solid dosage (e.g. freeze-dried) form. In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19) and poloxamer in a solid dosage (e.g. freeze-dried) form. Poloxamer may be present in an amount 0.1 to 0.5 mg, suitably 0.15 to 0.45 mg, 0.2 to 0.4 mg, 0.2 to 0.35 mg or 0.2 to 0.3 mg, e.g. 0.25 mg. Specifically, the poloxamer may be present in an amount 0.1 mg, 0.15 mg, 0.2 mg, 0.25 mg, 0.3 mg, 0.35 mg, 0.4 mg, 0.45 mg or 0.5 mg.

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188) and an adjuvant (e.g. AS01E). In another embodiment, the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and an adjuvant (e.g. AS01E). In another embodiment, the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and an adjuvant (e.g. AS01E). In another embodiment, the immunogenic composition comprises 20 μg/ml PE-PilA fusion protein (e.g. SEQ ID NO: 9), 20 μg/ml Protein D polypeptide (e.g. SEQ ID NO: 2), 6.6 μg/ml UspA2 polypeptide (e.g. SEQ ID NO: 19), 0.02 to 0.15% poloxamer (e.g. poloxamer 188) (w/v) and an adjuvant (e.g. AS01E). After reconstitution poloxamer may be present in an amount 0.02 to 0.15%, suitably 0.03 to 0.15%, 0.03 to 0.09%, 0.04 to 0.15%, 0.04 to 0.1%, 0.4 to 0.9%, or 0.03 to 0.05% (w/v). Specifically, the poloxamer may be present in an amount 0.03%, 0.04%, 0.05%, 0.06% or 0.07% w/v).

### Polysorbate 80

The present invention is also based, in part, on the identification of the need for and use of polysorbate 80 in an immunogenic composition comprising Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide. Polysorbate 80 (also known as PS80, Tween 80, sorbitan monooleate) is a non-ionic surfactant. Polysorbate 80 has been found according to the present invention to increase the stability of protein antigens. Surprisingly, the present inventors have found that the addition of polysorbate 80 (even a residual amount of polysorbate 80) further improves the stability of protein antigens in immunogenic compositions containing an antioxidant (e.g. L-methionine) and a polyoxamer (e.g. poloxamer 188). As described herein in the Examples (see Examples 5 and 6 herein), it was found that the stability of UspA2 polypeptide is increased in the presence of polysorbate 80, even at very low levels of polysorbate 80 such as a residual level of polysorbate 80 and even when Poloxamer 188 is already present as a surfactant. The formation of a pre-peak of UspA2 polypeptide during HP-SEC Fluo measurements (indicating lack of stability of the UspA2 polypeptide) and the improvement observed in the presence of polysorbate 80 was not expected. The present invention thus provides immunogenic compositions with improved stability. The present invention provides immunogenic compositions with improved stability compared to immunogenic compositions formulated without PS80. A residual amount of an excipient (e.g. polysorbate 80) is typically an amount lower than would normally be used in an immunogenic composition for the purpose of that excipient (e.g. in the case of polysorbate 80 a lower amount than would be normally required for the purpose of acting as a surfactant). For example, a residual amount of polysorbate 80 may be below the critical micellar concentration (the concentration at which the surfactant starts to form micelles (vesicles) rather than simply positioning itself on hydrophobic-hydrophylic interfaces), e.g. less than 0.05% (w/v), suitably less than 0.03% (w/v). The residual amount may be added during final formulation (mixing) of the immunogenic composition, but may also be present due to the inclusion of polysorbate 80 in processing steps prior to final formulation (mixing) of the immunogenic composition.

In immunogenic compositions of the present invention, polysorbate 80 may be present in a residual amount, e.g. less than 0.03% (w/v), e.g. 0.00001% to 0.03% (w/v) (such as an amount 0.0001% to 0.03% (w/v)). Suitably, the amount of polysorbate 80 in the immunogenic composition may be less than 0.03%, less than 0.025%, less than 0.02%, less than 0.015% or less than 0.01% (w/v). Preferably, in immunogenic compositions of the present invention, polysorbate 80 may be present in an amount 0.0001 to 0.03%, more preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

Thus, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188) and polysorbate 80. In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and 0.0001 to 0.02% polysorbate 80 (w/v), optionally in liquid form. In another embodiment, the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), and 0.0001% to 0.02% polysorbate 80 (w/v) optionally in a liquid form. In an embodiment, the immunogenic composition comprises 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19), 0.02 to 0.15% poloxamer (e.g. poloxamer 188) (w/v), and 0.0001% to 0.02% polysorbate 80 (w/v) optionally in a liquid form. Polysorbate 80 may be present in an amount 0.0001 to 0.03% (w/v), preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188) and polysorbate 80 in solid, e.g. freeze-dried form. In another embodiment, an immunogenic composition of the invention comprises 9 to 15 μg (e.g. 9 to 13 μg PE-PilA) fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19), poloxamer (e.g. 0.1 to 0.5 mg) and polysorbate 80 in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19), poloxamer and polysorbate 80 in a solid dosage (e.g. freeze-dried) form. In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19), poloxamer and polysorbate 80 in a solid dosage (e.g. freeze-dried) form. Polysorbate 80 may be present in an amount 1 to 50 μg. Polysorbate 80 may be present in an amount 1 to 10 μg, suitably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), polysorbate 80 and an adjuvant (e.g. AS01E). In another embodiment, the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and an adjuvant (e.g. AS01E). In another embodiment, the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 0.0001 to 0.02% polysorbate 80 (w/v) and an adjuvant (e.g. AS01E). In another embodiment, the immunogenic composition comprises 20 μg/ml PE-PilA fusion protein (e.g. SEQ ID NO: 9), 20 μg/ml Protein D polypeptide (e.g. SEQ ID NO: 2), 6.6 μg/ml UspA2 polypeptide (e.g. SEQ ID NO: 19), 0.02 to 0.15% poloxamer (e.g. poloxamer 188) (w/v), 0.0001 to 0.02% polysorbate 80 (w/v) and an adjuvant (e.g. AS01E). After reconstitution polysorbate 80 may be present in an amount 0.0001 to 0.02% (w/v), suitably 0.0001 to 0.01%. 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

### Anti-Oxidant

The present invention is also based, in part, on the identification of the need for and use of an antioxidant (e.g. L-methionine) in an immunogenic composition comprising a Protein D polypeptide, for example, immunogenic compositions comprising Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide from Moraxella catarrhalis, to mitigate oxidation of protein antigens. As described herein in the Examples, it was found that Protein D is susceptible to oxidation that may occur during formulation processes, e.g. freeze-drying (as measured by Methionine 192 oxidation) and the addition of an antioxidant, such as L-methionine, to the immunogenic composition can mitigate oxidation of Protein D which can occur during the formulation process, e.g. during freeze-drying. It was not previously known that Protein D is susceptible to oxidation during formulation processes and it was found that addition of an anti-oxidant provides an improved immunogenic composition compared to immunogenic compositions formulated without an anti-oxidant (see Examples 2 and 3 herein). An antioxidant for use in the compositions described herein is a pharmaceutically acceptable reagent that can be added to the formulation, to prevent or reduce oxidation of the protein antigen in the process or composition.

In an embodiment, the addition of an antioxidant prevents or reduces oxidation of the Protein D polypeptide. Methionine residues on a polypeptide or peptide such as a vaccine antigen may be vulnerable to oxidation for example oxidation due to the presence of hydrogen peroxide or simply by contact with ambient air or during a process such as lyophilization. Hydrogen peroxide may have been left over from the sterilisation of equipment used in the production of the biological medicament (residual hydrogen peroxide) and adsorbed or diffused into the formulation. The formulation may come into contact with air and/or be more vulnerable to oxidation for example during a process such as lyophilization where the formulation is freeze dried to produce a solid product (lyophilised cake).

In particular embodiments the antioxidant reduces oxidation of methionine groups on the Protein D polypeptide (e.g. Methionine 192, the amino acid corresponding to Methionine 192 in SEQ ID NO: 2). A person skilled in the art will understand that when the Protein D polypeptide sequence is a variant and/or fragment of an amino acid sequence of SEQ ID NO: 2, such as an amino acid sequence at least 80%, 85%, 90%, 92%, 95%, 96%, 97%, 98% or 99% identical to SEQ ID NO: 2, the reference to “Met192” (i.e. methionine 192) refers to a the position that would be equivalent to the defined position, if this sequence was lined up with an amino acid sequence of SEQ ID NO: 2 in order to maximise the sequence identity between the two sequences (Sequence alignment tools are not limited to Clustal Omega (www(.)ebi(.)ac(.)ac(.)uk) MUSCLE (www(.)ebi(.)ac(.)uk), or T-coffee (www(.)tcoffee(.)org). In one aspect, the sequence alignment tool used is Clustal Omega (www(.)ebi(.)ac(.)ac(.)uk).

In a particular embodiment the antioxidant reduces the oxidation of methionine groups to a level of no more than oxidation in the absence of hydrogen peroxide. In embodiments described herein, oxidation of polypeptides can be observed or measured by methods known in the art, such as those described herein in the Examples. Oxidation of proteins can be observed or measured by means of mass spectrometry, RP-HPLC and SDS-PAGE. In particular embodiments two of these three methods are used to observe or measure the level of oxidation, for example mass spectrometry and RP-HPLC. In another embodiment all three methods are used. In an embodiment, the antioxidant is an antioxidant that protects against oxidation of the biological molecule or vector without adversely affect the purity of the biological molecule or vector, for example it does not result in breakdown products detectable by RP-HPLC and/or LC-MS.

Examples of pharmaceutically acceptable antioxidants for use in immunogenic compositions described herein, include thiol containing excipients such as N-acetyl cysteine, L-cysteine, glutathione, monothioglycerol; and thioether containing excipients such as methionine, in the form of L-methionine or D-methionine; and ascorbic acid. Amino acid antioxidants such as methionine include monomeric or dimeric or multimeric forms of methionine or other amino acid, or amino acids present in mixed dimers or multimers such as methionine with one or more other another amino acids. Multimeric amino acids may contain for example up to three or four or five or six or seven or eight amino acids in total, which may be all the same for example all methionine, or all cysteine, or may be a mixture of amino acids including for example at least one methionine or cysteine, or predominantly for example methionine or cysteine or predominantly a mixture of methionine and cysteine. Short peptides of methionine or cysteine or short peptides of a mixture of methionine are included. In a particular embodiment, the antioxidant is a naturally occurring amino acid selected from L-methionine, L-cysteine and glutathione.

In another embodiment the antioxidant is L-methionine or L-cysteine. In particular embodiments the antioxidant is methionine (e.g. L-methionine). In particular embodiments the methionine is present in monomeric form.

In a particular embodiment the antioxidant (e.g. L-methionine) is present at a concentration between 0.05 mM to 50 mM in the immunogenic composition. In immunogenic compositions of the present invention, an antioxidant (optionally L-methionine) may be present in an amount 0.1 to 20 mM, 0.1 to 15 mM, or 0.5 to 15 mM, suitably 5 to 15 mM, 7 to 12 mM, 8 to 12 mM or 8 to 10 mM. Specifically, the concentration of antioxidant (e.g. L-methionine) may be 8 mM, 9 mM, 10 mM, 11 mM or 12 mM.

Suitably, immunogenic compositions of the invention comprise both an antioxidant (e.g. L-methionine) and poloxamer (e.g. poloxamer 188). In an embodiment, immunogenic compositions of the invention comprise 0.1 to 20 mM, 0.1 to 15 mM, or 0.5 to 15 mM, suitably 5 to 15 mM, 8 to 12 mM or 8 to 10 mM antioxidant (e.g. L-methionine) and 0.02 to 0.15%, suitably 0.03 to 0.15%, 0.03 to 0.09%, 0.04 to 0.15%, 0.04 to 0.1%, 0.04 to 0.09%, 0.04 to 0.06% or 0.04 to 0.05% (w/v) poloxamer (e.g. poloxamer 188). Preferably, immunogenic compositions of the invention comprise an antioxidant (e.g. L-methionine), poloxamer (e.g. poloxamer 188) and polysorbate 80. In an embodiment, immunogenic compositions of the invention comprise 0.1 to 20 mM, 0.1 to 15 mM, or 0.5 to 15 mM, suitably 5 to 15 mM, 8 to 12 mM or 8 to 10 mM antioxidant (e.g. L-methionine) and 0.02 to 0.15%, suitably 0.03 to 0.15%, 0.03 to 0.09%, 0.04 to 0.15%, 0.04 to 0.1%, 0.04 to 0.09%, 0.04 to 0.06% or 0.04 to 0.05% (w/v) poloxamer (e.g. poloxamer 188) and 0.0001 to 0.02%, suitably 0.0001 to 0.01%, 0.0001 to 0.005%, 0.0001 to 0.002%, 0.0002 to 0.002%, 0.0003 to 0.0015% or 0.0004 to 0.0012% (w/v) polysorbate 80.

Thus, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188) and an antioxidant (e.g. L-methionine). In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and 5 to 15 mM antioxidant (e.g. L-methionine), optionally in liquid form. In another embodiment, the immunogenic composition comprises 25 μg/ml PE-PilA fusion protein (e.g. SEQ ID NO: 9), 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and 0.1 to 20 mM methionine (e.g. L-methionine), optionally in a liquid form. In an embodiment, the immunogenic composition comprises 25 μg/ml PE-PilA fusion protein (e.g. SEQ ID NO: 9), 25 μg/ml Protein D polypeptide (e.g. SEQ ID NO: 2), and 8.3 μg/ml UspA2 polypeptide (e.g. SEQ ID NO: 19), 0.02 to 0.15% poloxamer (e.g. poloxamer 188) (w/v) and 0.1 to 20 mM methionine (e.g. L-methionine), optionally in a liquid form. The concentration of antioxidant (e.g. L-methionine) may be 0.1 to 20 mM, 0.1 to 15 mM, or 0.5 to 15 mM suitably 5 to 15 mM, 8 to 12 mM or 9 to 11 mM. Specifically, the concentration of antioxidant (e.g. L-methionine) may be 8 mM, 9 mM, 10 mM, 11 mM or 12 mM. Such immunogenic compositions may further comprise polysorbate 80 which may be present in an amount 0.0001 to 0.02% (w/v), suitably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188) and an antioxidant (e.g. L-methionine) in solid e.g. freeze-dried form. In another embodiment, an immunogenic composition of the invention comprises 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19), poloxamer (e.g. 0.1 to 0.5 mg) and antioxidant (e.g. L-methionine) in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19), poloxamer and antioxidant (e.g. L-methionine) in a solid dosage (e.g. freeze-dried) form. In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19), poloxamer and antioxidant (e.g. L-methionine) in a solid dosage (e.g. freeze-dried) form. Antioxidant (e.g. L-methionine) may be present in an amount 0.5 to 1.0 mg, suitably 0.6 to 0.9 mg, 0.7 to 0.8 mg, e.g. 0.75 mg. Specifically, the antioxidant (e.g. L-methionine) may be present in an amount 0.5 mg, 0.55 mg, 0.6 mg, 0.65 mg, 0.7 mg, 0.75 mg, 0.8 mg, 0.85 mg, 0.9 mg, 0.95 mg or 1.0 mg. In an embodiment, the concentration of the antioxidant in the freeze-dried immunogenic composition is less than the concentration of antioxidant prior to freeze-drying. Such immunogenic compositions may further comprise polysorbate 80 which may be present in an amount 1 to 50 μg, preferably 1 to 10 μg, more preferably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, the present invention provides an immunogenic composition comprising a PE-PilA fusion protein (e.g. SEQ ID NO: 9), an immunogenic fragment of Protein D (e.g. SEQ ID NO: 2), an immunogenic fragment of UspA2 (e.g. SEQ ID NO: 19), poloxamer (e.g. poloxamer 188) and antioxidant (e.g. L-methionine) and an adjuvant (e.g. AS01E). In another embodiment the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and 0.1 to 20 mM antioxidant (e.g. L-methionine) and an adjuvant (e.g. AS01E). In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v) and 0.1 to 20 mM antioxidant (e.g. L-methionine) and an adjuvant (e.g. AS01E). In another embodiment the immunogenic composition comprises 20 μg/ml PE-PilA fusion protein (e.g. SEQ ID NO: 9), 20 μg/ml Protein D polypeptide (e.g. SEQ ID NO: 2) and 6.6 μg/ml UspA2 polypeptide (e.g. SEQ ID NO: 19), 0.02 to 0.15% poloxamer (e.g. poloxamer 188) (w/v) and 0.1 to 20 mM antioxidant (e.g. L-methionine) and an adjuvant (e.g. AS01E). The concentration of antioxidant (e.g. L-methionine) after reconstitution may be 0.1 to 20 mM, 0.1 to 15 mM, or 0.5 to 15 mM suitably 5 to 15 mM, 6 to 10 mM or 7 to 9 mM. Specifically, the concentration of methionine after reconstitution may be 6 mM, 7 mM, 8 mM, 9 mM or 10 mM. Such immunogenic compositions may further comprise polysorbate 80 which may be present in an amount 0.0001 to 0.03% (w/v), preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

### Sucrose

An immunogenic composition of the invention may further comprise a sugar, optionally sucrose. Suitably, immunogenic compositions of the invention comprise sucrose in addition to an antioxidant (e.g. L-methionine) and poloxamer (e.g. poloxamer 188). Suitably, immunogenic compositions of the invention comprise sucrose in addition to an antioxidant (e.g. L-methionine), poloxamer (e.g. poloxamer 188) and polysorbate 80. In immunogenic compositions of the invention, sucrose may be present in an amount 1 to 10% suitably 3 to 7%, 3 to 6%, 4 to 6% or 4 to 5% (w/v). Specifically, the concentration of sucrose may be 3%, 4%, 5%, 6% or 7% (w/v).

Thus the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine) and sucrose. In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine) and 1 to 10% sucrose (w/v), optionally in liquid form. In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine) and 1 to 10% sucrose (w/v), optionally in a liquid form. In an embodiment the immunogenic composition comprises 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine) and 1 to 10% sucrose (w/v), optionally in a liquid form. The concentration of sucrose may be 1 to 10% suitably 3 to 7%, 3 to 6% or 4 to 6% (w/v). Specifically, the concentration of sucrose may be 3%, 4%, 5%, 6% or 7% (w/v). Such immunogenic compositions may further comprise polysorbate 80 which may be present in an amount 0.0001 to 0.02% (w/v), suitably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), an antioxidant (e.g. L-methionine) and sucrose in solid e.g. freeze-dried form. In another embodiment, an immunogenic composition of the invention comprises 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19), poloxamer (e.g. 0.1 to 0.5 mg), an antioxidant (e.g. L-methionine) and sucrose in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19), poloxamer, an antioxidant (e.g. L-methionine) and sucrose in a solid dosage (e.g. freeze-dried) form. In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19), poloxamer, an antioxidant (e.g. L-methionine) and sucrose in a solid dosage (e.g. freeze-dried) form. Sucrose may be present in an amount 10 to 50 mg, suitably 15 to 45 mg, 20 to 40 mg, 20 to 35 mg or 20 to 30 mg, e.g. 25 mg. Specifically, the sucrose may be present in an amount 10 mg, 15 mg, 20 mg, 25 mg, 30 mg, 35 mg, 40 mg, 45 mg or 50 mg. Such immunogenic compositions may further comprise polysorbate 80 which may be present in an amount 1 to 50 μg, preferably 1 to 10 μg, more preferably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine), sucrose and an adjuvant (e.g. AS01E). In another embodiment the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and an adjuvant (e.g. AS01E). In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and an adjuvant (e.g. AS01E). In another embodiment the immunogenic composition comprises 20 μg/ml PE-PilA fusion protein (e.g. SEQ ID NO: 9), 20 μg/ml Protein D polypeptide (e.g. SEQ ID NO: 2), and 6.6 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and an adjuvant (e.g. AS01E). The concentration of sucrose in the immunogenic composition (after reconstitution) may be 1 to 10% suitably 2 to 6%, 2 to 5% or 3 to 5% (w/v). Specifically, the concentration of sucrose after reconstitution may be 2%, 3%, 4%, 5% or 6% (w/v). Such immunogenic compositions may further comprise polysorbate 80 which may be present in an amount 0.0001 to 0.03% (w/v), preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

### Buffer

In an embodiment, the immunogenic composition of the invention comprises a buffer. Suitably, immunogenic compositions of the invention comprise a buffer in addition to sucrose, an antioxidant (e.g. L-methionine) and poloxamer (e.g. poloxamer 188). Suitably, immunogenic compositions of the invention comprise a buffer in addition to sucrose, an antioxidant (e.g. L-methionine), poloxamer (e.g. poloxamer 188) and polysorbate 80. In an embodiment, said buffer has a pKa of about 3.5 to about 7.5.

In some embodiments, the buffer is a phosphate, succinate, histidine or citrate buffer. In certain embodiments, the buffer is a phosphate buffer, suitably potassium phosphate (e.g. KH₂PO₄/K₂HPO₄).

In immunogenic compositions of the invention, the concentration of buffer may be 5 to 20 mM, suitably 10 to 15 mM, 10 to 14 mM or 10 to 13 mM. Specifically, the concentration of buffer may be 10.5 mM, 11.0 mM, 11.5 mM, 12.0 mM, 12.5 mM, 13.0 mM, 13.5 mM, 14.5 mM or 15.0 mM.

Thus, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine), sucrose and a buffer (e.g. phosphate buffer). In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and 5 to 20 mM buffer (e.g. phosphate buffer), optionally in liquid form. In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and 5 to 20 mM buffer (e.g. phosphate buffer), optionally in liquid form. In an embodiment the immunogenic composition comprises 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v) and 5 to 20 mM buffer (e.g. phosphate buffer), optionally in liquid form. The concentration of buffer may be 5 to 20 mM, suitably 10 to 15 mM, 11 to 14 mM or 12 to 13 mM. Specifically, the concentration of buffer may be 10.5 mM, 11.0 mM, 11.5 mM, 12.0 mM, 12.5 mM, 13.0 mM, 13.5 mM, 14.5 mM or 15.0 mM. Such immunogenic compositions may further comprise polysorbate 80 which may be present in an amount 0.0001 to 0.02% (w/v), suitably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), an antioxidant (e.g. L-methionine), sucrose and a buffer (e.g. phosphate buffer) in solid e.g. freeze-dried form. In another embodiment, an immunogenic composition of the invention comprises 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19), poloxamer (e.g. 0.1 to 0.5 mg), an antioxidant (e.g. L-methionine), sucrose and a buffer (e.g. phosphate buffer) in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19), poloxamer, an antioxidant (e.g. L-methionine), sucrose and a buffer (e.g. phosphate buffer) in a solid dosage (e.g. freeze-dried) form. In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19), poloxamer, an antioxidant (e.g. L-methionine), sucrose and a buffer (e.g. phosphate buffer) in a solid dosage (e.g. freeze-dried) form. Such immunogenic compositions may further comprise polysorbate 80 which may be present in an amount 1 to 50 μg, preferably 1 to 10 μg, more preferably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, the present invention provides an immunogenic composition comprising a PE-PilA fusion protein (e.g. SEQ ID NO: 8 or 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine), sucrose, buffer (e.g. phosphate buffer) and an adjuvant (e.g. AS01E). In another embodiment the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer (e.g. phosphate buffer) and an adjuvant (e.g. AS01E). In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer (e.g. phosphate buffer) and an adjuvant (e.g. AS01E). In another embodiment the immunogenic composition comprises 20 μg/ml PE-PilA fusion protein (e.g. SEQ ID NO: 9) 20 μg/ml Protein D polypeptide (e.g. SEQ ID NO: 2), 6.6 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer (e.g. phosphate buffer) and an adjuvant (e.g. AS01E). The concentration of buffer after reconstitution may be 5 to 20 mM, suitably 8 to 12 mM, 9 to 11 mM or 9.5 to 10.5 mM. Specifically, the concentration of buffer after reconstitution may be 8.0 mM, 8.5 mM, 9.0 mM, 9.5 mM, 10.0 mM, 10.5 mM, 11.0 mM. 11.5 mM or 12.0 mM. Such immunogenic compositions may further comprise polysorbate 80 which may be present in an amount 0.0001 to 0.03% (w/v), preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

pH

In an embodiment, the pH of the immunogenic composition of the invention may be pH5.0 to 9.0, pH5.5 to 8.5, pH6.0 to 8.0 or pH6.5 to 7.5. Specifically, the pH of the immunogenic composition of the invention may be pH6.5, pH6.6, pH6.7, pH6.8, pH6.9, pH7.0, pH7.1, pH7.2, pH7.3, pH7.4, or pH7.5. In another embodiment, the pH of an immunogenic composition of the invention (e.g. after reconstitution with adjuvant) may be pH6.0 to 8.0, pH6.1 to pH7.5, pH6.5 to 7.5, pH6.5 to 7.0 or pH6.5 to pH6.7. Specifically, the pH of an immunogenic composition of the invention (e.g. after reconstitution with adjuvant) may be pH6.5, pH6.6, pH6.7, pH6.8, pH6.9, pH7.0, pH7.1, pH7.2, pH7.3, pH7.4, or pH7.5.

### Additional Components

Residual material from individual antigenic components may also be present in trace amounts in the final vaccine produced by the process of the invention. For example, if arginine is used during antigen preparation then the final vaccine product may contain trace amounts of arginine. Thus, in an embodiment, the immunogenic composition of the invention comprises arginine. If salts have been used during antigen preparation (e.g. NaCl), then the final vaccine product may contain trace amounts of salt, (e.g. NaCl). Thus, in an embodiment the immunogenic composition of the invention comprises salt e.g. sodium chloride, calcium chloride, or sodium phosphate. In another embodiment the immunogenic composition of the invention comprises NaCl (sodium chloride). In addition to residual amounts of arginine and/or salt, immunogenic compositions of the invention may comprise a residual amount of polysorbate 80.

Thus, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine), sucrose, a buffer, arginine and a salt (e.g. NaCl). In an embodiment the immunogenic composition comprises 20 to 30 μg/ml PE-PilA fusion protein (e.g. SEQ ID NO: 9), 20 to 30 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM NaCl, optionally in liquid form. In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM NaCl, optionally in liquid form. In an embodiment the immunogenic composition comprises 25 μg/ml PE-PilA fusion protein (e.g. SEQ ID NO: 9), 25 μg/ml Protein D polypeptide (e.g. SEQ ID NO: 2), 8.3 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM NaCl, optionally in liquid form. The concentration of arginine may be 1 to 5 mM, suitably 1 to 4 mM or 2 to 4 mM. Specifically, the final concentration of arginine may be 1 mM, 2 mM, 3 mM, 4 mM or 5 mM. The concentration of salt (e.g. NaCl) may be 1 to 10 mM, suitably 2 to 7 mM, 3 to 6 mM or 4 to 5 mM. Specifically, the concentration of salt (e.g. NaCl) may be 1 mM, 2 mM, 3 mM, 4 mM, 5 mM, 6 mM or 7 mM. Such immunogenic compositions may further comprise polysorbate 80 which may be present in an amount 0.0001 to 0.03% (w/v), preferably 0.0001 to 0.02% (w/v), more preferably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), an antioxidant (e.g. L-methionine), sucrose, a buffer, arginine and a salt (e.g. NaCl) in solid e.g. freeze-dried form. In another embodiment, an immunogenic composition of the invention comprises 9 to 15 μg (e.g. 9 to 13 μg) PE-PilA fusion protein (e.g. SEQ ID NO: 9), 9 to 15 μg (e.g. 9 to 13 μg) Protein D polypeptide (e.g. SEQ ID NO: 2), 3 to 5 μg UspA2 polypeptide (e.g. SEQ ID NO: 19), poloxamer (e.g. 0.1 to 0.5 mg), an antioxidant (e.g. L-methionine), sucrose, a buffer, arginine and a salt (e.g. NaCl) in a solid dosage (e.g. freeze-dried) form. In another embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19), poloxamer, an antioxidant (e.g. L-methionine), sucrose, a buffer, arginine and a salt (e.g. NaCl) in a solid dosage (e.g. freeze-dried) form.

In another embodiment, an immunogenic composition of the invention comprises 12.5 μg PE-PilA fusion protein (e.g. SEQ ID NO: 9), 12.5 μg Protein D polypeptide (e.g. SEQ ID NO: 2), 4.15 μg UspA2 polypeptide (e.g. SEQ ID NO: 19), poloxamer, an antioxidant (e.g. L-methionine), sucrose, a buffer, arginine and a salt (e.g. NaCl) in a solid dosage (e.g. freeze-dried) form. Such immunogenic compositions may further comprise polysorbate 80 which may be present in an amount 1 to 50 μg, preferably 1 to 10 μg, more preferably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, the present invention provides an immunogenic composition comprising a PE-PilA fusion protein (e.g. SEQ ID NO: 9), a Protein D polypeptide (e.g. SEQ ID NO: 2), an UspA2 polypeptide (e.g. SEQ ID NO: 19), poloxamer (e.g. poloxamer 188), antioxidant (e.g. L-methionine), sucrose, a buffer, arginine and a salt (e.g. NaCl) and an adjuvant (e.g. AS01E). In another embodiment the immunogenic composition comprises 15 to 25 μg/ml PE-PilA fusion protein (e.g. SEQ ID NO: 9), 15 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM salt (e.g. NaCl) and an adjuvant (e.g. AS01E). In another embodiment the immunogenic composition comprises 20 to 25 μg/ml PE-PilA fusion protein (e.g. SEQ ID NO: 9), 20 to 25 μg/ml Protein D polypeptide (e.g. SEQ ID NO: 2), 6 to 9 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM salt (e.g. NaCl) and an adjuvant (e.g. AS01E). In another embodiment the immunogenic composition comprises 20 μg/ml PE-PilA fusion protein (e.g. SEQ ID NO: 9), 20 μg/ml Protein D polypeptide (e.g. SEQ ID NO: 2), 6.6 μg/ml UspA2 polypeptide (e.g. SEQ ID NO: 19), 0.02 to 0.15% poloxamer (e.g. poloxamer 188) (w/v), 5 to 15 mM antioxidant (e.g. L-methionine), 1 to 10% sucrose (w/v), 5 to 20 mM buffer, 1 to 5 mM arginine and 1 to 10 mM salt (e.g. NaCl) and an adjuvant (e.g. AS01E). The concentration of arginine after reconstitution may be 1 to 4 mM, suitably 1 to 3 mM or 2 to 3 mM. Specifically, the concentration of arginine after reconstitution may be 1 mM, 2 mM, 3 mM, 4 mM or 5 mM. The concentration of salt (e.g. NaCl) after reconstitution may be 1 to 10 mM, suitably 1 to 6 mM or 2 to 5 mM. Specifically, the concentration of salt (e.g. NaCl) may be 1 mM, 2 mM, 3 mM, 4 mM, 5 mM, 6 mM or 7 mM. Such immunogenic compositions may further comprise polysorbate 80 which may be present in an amount 0.0001 to 0.02% (w/v), suitably 0.0001 to 0.01%, 0.0001 to 0.005%, 0.0001 to 0.002%, 0.0002 to 0.002%, 0.0003 to 0.0015% or 0.0004 to 0.0012% (w/v). Specifically, polysorbate 80 may be present in an amount 0.0003 to 0.002%, 0.0003 to 0.0009%, 0.0003 to 0.0006%, 0.0006 to 0.002%, or 0.0006 to 0.0009% (w/v).

### Presentation

In certain embodiments, the immunogenic composition is contained within a container means e.g. a vial, or a syringe, including a pre-filled syringe. In certain embodiments, the container is siliconized.

Where an immunogenic composition of the invention is presented in a vial, this is suitably made of a glass or plastic material. The vial is preferably sterilized before the composition is added to it. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be withdrawn back into the syringe. After removal of the syringe from the vial, a needle can then be attached and the composition can be administered to a patient. The cap is preferably located inside a seal or cover, such that the seal or cover has to be removed before the cap can be accessed.

Immunogenic compositions of the invention may be adapted for administration by an appropriate route, for example, by the intramuscular route.

The present invention provides for the first time a kit comprising an immunogenic composition of the invention in solid form (e.g. freeze-dried). In another embodiment, the present invention provides a kit comprising (i) a first container (optionally a vial) comprising an immunogenic composition of the invention, optionally in solid form (optionally freeze-dried) and (ii) a second container (optionally a pre-filled syringe) comprising an adjuvant, optionally AS01E. The contents of the second container may be used to reconstitute the immunogenic composition in the first container prior to administration.

In another embodiment, the present invention provides a vaccine comprising an immunogenic composition of the invention.

### Pharmaceutically Acceptable Excipients

Immunogenic compositions of the invention may further comprise additional pharmaceutically acceptable excipient(s). Possible excipients include diluents such as water, saline, glycerol etc. Additionally, auxiliary substances, such as wetting or emulsifying agents, pH buffering substances, polyols and the like may be present. For example, immunogenic compositions of the invention may comprise water for injection (WFI).

### Process for Preparing an Immunogenic Composition of the Invention

The present invention provides a process for preparing an immunogenic composition of the present invention. In an embodiment, the present invention provides a process for preparing an immunogenic composition of the invention comprising combining Protein E from Haemophilus influenzae or an immunogenic fragment thereof and PilA from Haemophilus influenzae or an immunogenic fragment thereof, optionally as a fusion protein (optionally a PE-PilA fusion protein); a Protein D polypeptide; an UspA2 polypeptide; with an anti-oxidant (optionally L-methionine) and poloxamer (optionally poloxamer 188).

According to the present invention, it has been found that the use of poloxamer is advantageous in the process for preparing an immunogenic composition of the present invention. It has been found that the addition of poloxamer limits the volume that needs to be discarded after the filtration step as it reduces aspecific adsorption. Thus, the present invention provides an improved process for preparing immunogenic compositions comprising Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; and an UspA2 polypeptide. It has also been found that the use of polysorbate 80 is advantageous in the process for preparing an immunogenic composition of the present invention. The inclusion of polysorbate 80 in the preparation of the immunogenic composition has been found to improve the stability of the UspA2 polypeptide compared to an immunogenic composition prepared without polysorbate 80.

The immunogenic composition of the invention may be provided in solid form, e.g. freeze dried. The immunogenic composition in solid form may be obtained from a liquid composition, for example by freeze drying or spray-freeze drying. Thus, in another embodiment, the present invention provides a process for preparing an immunogenic composition of the invention comprising combining Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide; with an anti-oxidant (e.g. L-methionine) and poloxamer (e.g. poloxamer 188) and freeze drying the immunogenic composition. In a further embodiment, the present invention provides a process for preparing an immunogenic composition of the invention comprising combining Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide; with an anti-oxidant (e.g. L-methionine), poloxamer (e.g. poloxamer 188) and polysorbate 80 and freeze drying the immunogenic composition. In a further embodiment, the present invention provides a process for preparing an immunogenic composition of the invention comprising combining the antigens Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide; with an anti-oxidant (e.g. L-methionine), poloxamer (e.g. poloxamer 188) and polysorbate 80, wherein polysorbate 80 is added during the preparation of the antigens (e.g. during preparation of the PE-PilA fusion protein antigen) and poloxamer (e.g. poloxamer 188) is added during the step of mixing the antigens, and freeze drying the immunogenic composition.

“Freeze-drying” refers to the process by which a suspension is frozen, after which the water is removed by sublimation. Sublimation is a change in the physical properties of a composition, wherein the solvent, e.g. water, in the substance changes directly from a solid (frozen) state to a gaseous state without becoming a liquid. Freeze drying is a low temperature dehydration process which involves freezing the formulation (e.g. an aqueous formulation) to below the triple point (the lowest temperature at which the solid, liquid and gas phases of the material can coexist), lowering pressure and removing ice (solid solvent) by sublimation in a primary drying step and removing remaining water in a second drying step. Annealing may optionally be used prior to drying to increase the size of the ice crystals by raising and lowering the temperature. Annealing is carried out by maintaining the temperature over the glass transition temperature (Tg′) of the formulation, maintaining it for a certain amount of time, before decreasing it below the Tg′. Controlled-nucleation may also be used to increase the size of the ice crystals, with the same effect on the matrix. Lyophilization is commonly used in vaccine manufacturing.

In an embodiment, the immunogenic composition is lyophilized. Lyophilization is the process by which water is removed from a product after it is frozen and placed under a vacuum, allowing the ice to change directly from solid to vapor without passing through a liquid phase.

In an embodiment lyophilization is carried out using the following steps:
a freezing step (below the triple point)
optionally an annealing step or a controlled nucleation step
a primary drying step
a secondary drying step.
   Lyophilization increases the concentration of components of a formulation in a process known as cryoconcentration. The resulting increase in concentration of residual hydrogen peroxide described herein may cause or accentuate a deleterious effect of the hydrogen peroxide such as oxidation of biological components, e.g. polypeptides in the immunogenic composition. The present invention provides immunogenic composition which reduces the oxidation of antigenic polypeptides (e.g. Protein D) which may occur during formulation (e.g. freeze drying/lyophilization). In an embodiment, the concentration of the antioxidant in the freeze-dried immunogenic composition is less than the concentration of antioxidant in the liquid composition prior to freeze-drying.
   Thus, in an embodiment, the present invention provides a process for preparing an immunogenic composition of the invention comprising combining Protein E from Haemophilus influenzae or an immunogenic fragment thereof and PilA from Haemophilus influenzae or an immunogenic fragment thereof, optionally as a fusion protein (e.g. a PE-PilA fusion protein); a Protein D polypeptide; an UspA2 polypeptide; with an anti-oxidant (e.g. L-methionine) and poloxamer (e.g. poloxamer 188) and subsequently freeze-drying the immunogenic composition. In a embodiment, the freeze-dried composition produced by the process of the invention is suitable for reconstitution in an aqueous solution (e.g. comprising an adjuvant, e.g. AS01E) prior to administration.

### Reconstituted Immunogenic Compositions

The immunogenic compositions of the invention in solid form (e.g. freeze-dried) are suitably intended for reconstitution in an aqueous solution (e.g. an aqueous solution comprising an adjuvant e.g. AS01E). Thus the present invention provides an immunogenic composition suitable for reconstitution in an aqueous solution (optionally in an aqueous solution comprising an adjuvant e.g. AS01E). In an embodiment, the immunogenic composition after reconstitution is capable of generating an immune response against Haemophilus influenzae and/or Moraxella catarrhalis. In an embodiment, the immunogenic composition after reconstitution is capable of generating an immune response against Haemophilus influenzae and Moraxella catarrhalis. Suitably, in the immunogenic composition of the invention the level of oxidation of Protein D polypeptide does not increase overtime, e.g. during storage. Suitably, in the immunogenic composition of the invention the Protein D polypeptide is not oxidised. Suitably, in the immunogenic compositions of the invention the Protein D polypeptide is not aggregated such that particles are visible. Suitably, in the immunogenic compositions of the invention the level of aggregation of PE-PilA and/or UspA2 polypeptide does not increase over time, e.g. during storage. Suitably, in the immunogenic compositions of the invention the PE-PilA and/or UspA2 polypeptide are not aggregated. Suitably, in the immunogenic compositions of the invention the protein antigens: Protein D polypeptide, PE-PilA and UspA2 polypeptide are stable. The following immunogenic compositions may further comprise polysorbate 80.

Immunogenic compositions of the invention in solid form (e.g. freeze-dried) as described above may be reconstituted with WFI and/or an adjuvant (e.g. AS01E) prior to vaccine administration. Thus, the present invention also provides an immunogenic composition in a liquid form reconstituted with an aqueous solution, optionally comprising an adjuvant e.g. AS01E. In an embodiment, an immunogenic composition of the invention comprises 15 to 25 μg/ml PE-PilA fusion protein; 15 to 25 μg/ml Protein D polypeptide; 6 to 9 μg/ml UspA2 polypeptide; 0.5 to 1.5 mg/ml, e.g. 1.2 mg/ml antioxidant (e.g. L-methionine); 0.2 to 0.6 mg/ml e.g. 0.4 mg/ml poloxamer (e.g. poloxamer 188); 20 to 60 mg/ml, e.g. 40 mg/ml sucrose; and optionally an adjuvant, e.g. AS01E, optionally in a 0.5 ml dose. In an embodiment, an immunogenic composition of the invention comprises 20 to 25 μg/ml PE-PilA fusion protein; 20 to 25 μg/ml Protein D polypeptide; 6 to 9 μg/ml UspA2 polypeptide; 0.5 to 1.5 mg/ml, e.g. 1.2 mg/ml antioxidant (e.g. L-methionine); 0.2 to 0.6 mg/ml e.g. 0.4 mg/ml poloxamer (e.g. poloxamer 188); 20 to 60 mg/ml, e.g. 40 mg/ml sucrose; and 5 to 20 mM e.g. 10 mM buffer (e.g. phosphate buffer) and optionally an adjuvant, e.g. AS01E, optionally in a 0.5 ml dose. In another embodiment, an immunogenic composition of the invention comprises 20 μg/ml PE-PilA fusion protein; 20 μg/ml Protein D polypeptide; 6.6 μg/ml UspA2 polypeptide; 0.5 to 1.5 mg/ml, e.g. 1.2 mg/ml antioxidant (optionally L-methionine); 0.2 to 0.6 mg/ml e.g. 0.4 mg/ml poloxamer (optionally poloxamer 188); 20 to 60 mg/ml, e.g. 40 mg/ml sucrose; and optionally an adjuvant, e.g. AS01E, optionally in a 0.5 ml dose.

Immunogenic compositions of the invention may be administered in a 0.5 ml dose. Thus, an immunogenic composition of the invention may comprise 9 to 13 μg PE-PilA fusion protein (e.g. SEQ ID NO: 9), 9 to 13 μg Protein D polypeptide (e.g. SEQ ID NO: 2), 3 to 5 μg UspA2 polypeptide (e.g. SEQ ID NO: 19); and optionally an adjuvant, e.g. AS01E, optionally in a 0.5 ml dose. In an embodiment, an immunogenic composition of the invention comprises 10 to 12.5 μg PE-PilA fusion protein (e.g. SEQ ID NO: 9), 10 to 12.5 μg Protein D polypeptide (e.g. SEQ ID NO: 2), 3 to 5 μg UspA2 polypeptide (e.g. SEQ ID NO: 19); and optionally an adjuvant, e.g. AS01E, optionally in a 0.5 ml dose. In another embodiment, an immunogenic composition of the invention may comprises 10 μg PE-PilA fusion protein (e.g. SEQ ID NO: 9), 10 μg Protein D polypeptide (e.g. SEQ ID NO: 2), 3.3 μg UspA2 polypeptide (e.g. SEQ ID NO: 19); and optionally an adjuvant, e.g. AS01E, optionally in a 0.5 ml dose. In another embodiment, an immunogenic composition of the invention may comprise 10 μg PE-PilA fusion protein; 10 μg Protein D polypeptide; 3.3 μg UspA2 polypeptide from Moraxella catarrhalis; 0.25 to 0.75 mg, e.g. 0.6 mg antioxidant (e.g. L-methionine); 0.1 to 0.3 mg e.g. 0.2 mg poloxamer (e.g. poloxamer 188); and 10 to 30 mg, e.g. 20 mg sucrose; and optionally an adjuvant, e.g. AS01E, optionally in a 0.5 ml dose. In another embodiment, an immunogenic composition of the invention may comprise 10 μg PE-PilA fusion protein; 10 μg Protein D polypeptide; 3.3 μg UspA2 polypeptide; 0.25 to 0.75 mg, e.g. 0.6 mg antioxidant (e.g. L-methionine); 0.1 to 0.3 mg e.g. 0.2 mg poloxamer (e.g. poloxamer 188); and 10 to 30 mg, e.g. 20 mg sucrose; and 5 to 20 mM e.g. 10 mM buffer (e.g. phosphate buffer); and optionally an adjuvant, e.g. AS01E, optionally in a 0.5 ml dose. Such immunogenic compositions may further comprise polysorbate 80 which may be present in an amount 1 to 10 μg, suitably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

In another embodiment, an immunogenic composition of the invention may comprise 10 μg PE-PilA fusion protein; 10 μg Protein D polypeptide; 3.3 μg UspA2 polypeptide; 0.25 to 0.75 mg, e.g. 0.6 mg antioxidant (e.g. L-methionine); 0.1 to 0.3 mg e.g. 0.2 mg poloxamer (e.g. poloxamer 188); and 10 to 30 mg, e.g. 20 mg sucrose; 5 to 20 mM e.g. 10 mM buffer (e.g. phosphate buffer) and salt (e.g. NaCl). In another embodiment, an immunogenic composition of the invention may comprise 10 μg PE-PilA fusion protein; 10 μg Protein D polypeptide; 3.3 μg UspA2 polypeptide; 0.25 to 0.75 mg, e.g. 0.6 mg antioxidant (e.g. L-methionine); 0.1 to 0.3 mg e.g. 0.2 mg poloxamer (e.g. poloxamer 188); and 10 to 30 mg, e.g. 20 mg sucrose; 5 to 20 mM e.g. 10 mM buffer (e.g. phosphate buffer), salt (e.g. NaCl) and arginine. Such immunogenic compositions may further comprise polysorbate 80 which may be present in an amount 1 to 10 μg, suitably 1 to 6 μg, 1 to 5 μg, 2 to 4 μg or 3 to 4 μg, e.g. 3.2 μg. Specifically, the polysorbate 80 may be present in an amount 1 μg, 1.5 μg, 2 μg, 2.5 μg, 3 μg, 3.5 μg, 4 μg, 4.5 μg or 5 μg.

### Uses, Methods of Treatment and Prevention

The present invention provides an immunogenic composition for use in the treatment or prevention of a disease caused by H. influenzae and/or M. catarrhalis. The present invention also provides an immunogenic composition of the invention (or a kit of the invention) for use in the treatment or prevention of an acute exacerbation of COPD (AECOPD) in a subject, e.g. human. The present invention provides an immunogenic composition for use in the treatment or prevention of a disease caused by H. influenzae and/or M. catarrhalis and the treatment or prevention of an acute exacerbation of COPD (AECOPD) in a subject, e.g. human.

The present invention provides use of an immunogenic composition of the invention, in the manufacture of a medicament for the treatment or prevention of a disease caused by H. influenzae and/or M. catarrhalis. The present invention also provides use of an immunogenic composition of the invention (or a kit of the invention), in the manufacture of a medicament for the treatment or prevention of an acute exacerbation of COPD (AECOPD) in a subject, e.g. human. The present invention provides use of an immunogenic composition of the invention, in the manufacture of a medicament for the treatment or prevention of a disease caused by H. influenzae and/or M. catarrhalis and the treatment or prevention of an acute exacerbation of COPD (AECOPD) in a subject, e.g. human.

The present invention provides a method of treatment or prevention of a disease caused by H. influenzae and/or M. catarrhalis, in a subject, e.g. human, at risk, said method comprising administering to said subject, an effective amount of an immunogenic composition of the invention. The present invention also provides a method of treatment or prevention of an acute exacerbation of COPD (AECOPD) in a subject, e.g. human, at risk of developing an acute exacerbation of COPD (AECOPD), said method comprising administering to said subject, an effective amount of an immunogenic composition of the invention. The present invention provides a method of treatment or prevention of a disease caused by H. influenzae and/or M. catarrhalis and the treatment or prevention of an acute exacerbation of COPD (AECOPD), in a subject, e.g. human, at risk, said method comprising administering to said subject, an effective amount of an immunogenic composition of the invention. The present invention provides a method of prevention of a disease caused by H. influenzae and/or M. catarrhalis, in a subject, e.g. human, at risk, said method comprising administering to said subject, an effective amount of an immunogenic composition of the invention. The present invention also provides a method of prevention of an acute exacerbation of COPD (AECOPD) in a subject, e.g. human, at risk of developing an acute exacerbation of COPD (AECOPD), said method comprising administering to said subject, an effective amount of an immunogenic composition of the invention.

The present invention provides a method of treatment of a disease caused by H. influenzae and/or M. catarrhalis, in a subject, e.g. human, at risk, said method comprising administering to said subject, an effective amount of an immunogenic composition of the invention. The present invention also provides a method of treatment of an acute exacerbation of COPD (AECOPD) in a subject, e.g. human, at risk of developing an acute exacerbation of COPD (AECOPD), said method comprising administering to said subject, an effective amount of an immunogenic composition of the invention.

The present invention provides a method of inducing an immune response to H. influenzae and/or M. catarrhalis in a subject (e.g. human), said method comprising administering to said subject, an effective amount of an immunogenic composition of the invention.

Chronic obstructive pulmonary disease (COPD) is a lung disease characterized by chronic obstruction of lung airflow that interferes with normal breathing and is not fully reversible. A COPD diagnosis is confirmed by a simple test called spirometry, which measures how deeply a person can breathe and how fast air can move into and out of the lungs. Such a diagnosis should be considered in any patient who has symptoms of cough, sputum production, or dyspnea (difficult or labored breathing), and/or a history of exposure to risk factors for the disease. Where spirometry is unavailable, the diagnosis of COPD should be made using all available tools. Clinical symptoms and signs, such as abnormal shortness of breath and increased forced expiratory time, can be used to help with the diagnosis. A low peak flow is consistent with COPD, but may not be specific to COPD because it can be caused by other lung diseases and by poor performance during testing. Chronic cough and sputum production often precede the development of airflow limitation by many years, although not all individuals with cough and sputum production go on to develop COPD.

An acute exacerbation of COPD (AECOPD) is an acute event characterised by a worsening of the patient's respiratory symptoms that is beyond normal day-to-day variations. Typically an AECOPD leads to a change in medication. Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication [Perez A C, Murphy T F. Potential impact of a Moraxella catarrhalis vaccine in COPD. Vaccine. 2017]. AECOPD increases morbidity and mortality, leading to faster decline in lung function, poorer functional status [Sapey E, Stockley R A. COPD exacerbations. 2: aetiology. Thorax. 2006; 61(3):250-8)]. The lungs are known to be colonised with different species of bacteria [Erb-Downward J R, et al. PLoS One. 2011; 6(2):e16384 and Wilkinson T M A, et al. Thorax. 2017; 72(10):919-27]. In COPD patients, acquisition of new bacterial strains is believed to be an important cause of AECOPD [Sethi S, et al. N Engl J Med. 2002; 347(7):465-71]. Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) [Alamoudi O S. et al. Respirology. 2007; 12(2):283-7, Bandi V, et al. FEMS Immunol Med Microbiol. 2003; 37(1):69-75, Beasley V, et al. Int J Chron Obstruct Pulmon Dis. 2012; 7:555-69]. In an embodiment, the acute exacerbation of chronic obstructive pulmonary disease (AECOPD) is associated with a bacterial infection in a subject, e.g. a bacterial infection of Haemophilus influenzae (e.g. non-typeable H. influenzae (NTHi)) and/or Moraxella catarrhalis. In another embodiment, the bacterial infection is present in the lung(s) of a subject, e.g. human. In another embodiment, the subject, e.g. human, is at risk for developing an acute exacerbation of chronic obstructive pulmonary disease (AECOPD) resulting from a bacterial infection.

### Analytical Methods

Purity of a sample was assessed by HPSEC-Fluo, also referred to as “SEC-HPLC-Fluo”, high-performance size exclusion chromatography with fluorescence detection. HPSEC is a special type of liquid chromatography that separates molecules based on molecular sizes or hydrodynamic volumes and not according to partition or affinities toward the stationary phases.

Antigen content was assessed by Reverse Phase-Ultra High Performance Chromatography (RP-UPLC) Reversed phase HPLC (RP-HPLC) uses a non-polar stationary phase and an aqueous, moderately polar mobile phase. With such stationary phases, retention time is longer for molecules which are less polar, while polar molecules elute more readily (early in the analysis). RP-HPLC is carried out under denaturing conditions with a slow gradient enabling the separation of hydrophobic variants.

Antigenic activity was assessed by Enzyme Linked Immunosorbent Assay (ELISA) The assay uses a solid-phase enzyme immunoassay (EIA) to detect the presence of a ligand (commonly a protein) in a liquid sample using antibodies directed against the protein to be measured. The sample with an unknown amount of antigen is immobilized on a solid support via capture by another antibody specific to the same antigen, in a “sandwich” ELISA)

Embodiments of the invention are further described in the subsequent numbered paragraphs:

1. An immunogenic composition comprising Protein E from Haemophilus influenzae or an immunogenic fragment thereof and PilA from Haemophilus influenzae or an immunogenic fragment thereof, optionally as a fusion protein (optionally a PE-PilA fusion protein, e.g. SEQ ID NO: 9); a Protein D polypeptide (optionally a Protein D polypeptide of SEQ ID NO: 2); an UspA2 polypeptide (optionally an UspA2 polypeptide of SEQ ID NO: 19); an anti-oxidant (optionally L-methionine) and poloxamer (optionally poloxamer 188).
2. The immunogenic composition according to paragraph 1, wherein the Protein E from Haemophilus influenzae or an immunogenic fragment thereof has at least 70%, 80%, 85%, 90%, 91%, 92%, 93%, 94%, 95%, 96%, 97%, 98%, 99%, or 100% identity to SEQ ID NO: 5.
3. The immunogenic composition according to paragraph 1 or paragraph 2, wherein the PilA from Haemophilus influenzae or an immunogenic fragment thereof has at least 70%, 80%, 85%, 90%, 91%, 92%, 93%, 94%, 95%, 96%, 97%, 98%, 99%, or 100% identity to SEQ ID NO: 7.
4. The immunogenic composition according to any of paragraphs 1 to 3, wherein the Protein E from Haemophilus influenzae or an immunogenic fragment thereof and the PilA from Haemophilus influenzae or an immunogenic fragment thereof are presented as a fusion protein (optionally a PE-PilA fusion protein), optionally having at least 70%, 80%, 85%, 90%, 91%, 92%, 93%, 94%, 95%, 96%, 97%, 98%, 99%, or 100% identity to SEQ ID NO: 9.
5. The immunogenic composition according to any of paragraphs 1 to 4, wherein the Protein D polypeptide has at least 70%, 80%, 85%, 90%, 91%, 92%, 93%, 94%, 95%, 96%, 97%, 98%, 99%, or 100% identity to SEQ ID NO: 2.
6. The immunogenic composition according to any of paragraphs 1 to 5, wherein the UspA2 polypeptide has at least 70%, 80%, 85%, 90%, 91%, 92%, 93%, 94%, 95%, 96%, 97%, 98%, 99%, or 100% identity to polypeptide MC009 (SEQ ID NO: 19).
7. The immunogenic composition according to any of paragraphs 1 to 6 comprising a PE-PilA fusion protein (optionally a PE-PilA fusion protein of SEQ ID NO: 9), a Protein D polypeptide (optionally a Protein D polypeptide of SEQ ID NO: 2), an UspA2 polypeptide (optionally an UspA2 polypeptide of SEQ ID NO: 19), an anti-oxidant (optionally L-methionine) and poloxamer (optionally poloxamer 188).
8. The immunogenic composition according to any of paragraphs 1 to 7, comprising 0.02 to 0.15%, 0.03 to 0.15%, 0.03 to 0.09%, 0.04 to 0.15%, 0.04 to 0.1%, 0.04 to 0.09%, 0.04 to 0.06% or 0.04 to 0.05% (w/v) poloxamer (optionally poloxamer 188).
10. The immunogenic composition according to any of paragraphs 1 to 8, comprising 0.1 to 20 mM, 0.1 to 15 mM, 0.5 to 15 mM, 5 to 15 mM, 8 to 12 mM or 8 to 10 mM antioxidant (optionally L-methionine).
11. The immunogenic composition according to any of paragraphs 1 to 10, comprising 20 to 30 μg/ml PE-PilA fusion protein, 20 to 30 μg/ml Protein D polypeptide and 6 to 9 μg/ml UspA2 polypeptide.
12. The immunogenic composition according to any of paragraphs 1 to 11, comprising 9 to 15 μg (e.g.
9 to 13 μg) PE-PilA fusion protein (optionally a PE-PilA fusion protein of SEQ ID NO: 9), 9 to 15 μg (e.g.
9 to 13 μg) Protein D polypeptide (optionally a Protein D polypeptide of SEQ ID NO: 2), 3 to 5 μg UspA2 polypeptide (optionally an UspA2 polypeptide of SEQ ID NO: 19).
13. The immunogenic composition according to any of paragraphs 1 to 12, further comprising polysorbate 80, optionally comprising 0.0001 to 0.03%, suitably 0.0001 to 0.02%, e.g. 0.0001 to 0.01%, 0.0001 to 0.005%, 0.0001 to 0.002%, 0.0002 to 0.002%, 0.0003 to 0.0015% or 0.0004 to 0.0012% (w/v) polysorbate 80.
14. The immunogenic composition according to any of paragraphs 1 to 13, in a solid form (optionally freeze-dried).
15. The immunogenic composition according to paragraph 14, suitable for reconstitution in an aqueous solution (optionally in an aqueous solution comprising an adjuvant e.g. AS01E), wherein said immunogenic composition after reconstitution is capable of generating an immune response against Haemophilus influenzae and/or Moraxella catarrhalis.
16. The immunogenic composition according to any of paragraphs 1 to 13, in a liquid form (optionally reconstituted with an aqueous solution comprising an adjuvant e.g. AS01E).
17. The immunogenic composition according to any of paragraphs 1 to 7, comprising 0.1 to 20 mM, 0.1 to 15 mM, or 0.5 to 15 mM, suitably 5 to 15 mM, 6 to 10 mM or 7 to 9 mM, e.g. 8 mM antioxidant (optionally L-methionine); and 0.02 to 0.15%, suitably 0.03 to 0.15%, 0.03 to 0.09%, 0.04 to 0.15%, 0.04 to 0.1%, 0.4 to 0.9%, or 0.03 to 0.05%, e.g. 0.04% (w/v) poloxamer (optionally poloxamer 188).
18. The immunogenic composition according to any of paragraphs 1 to 7 or 17, comprising 20 μg/ml PE-PilA fusion protein, 20 μg/ml Protein D polypeptide, 6.6 μg/ml UspA2 polypeptide.
19. The immunogenic composition according to any of paragraphs 1 to 13, 17 or 18, further comprising an adjuvant, optionally AS01E.
20. The immunogenic composition according to any of paragraphs 1 to 19, further comprising sucrose.
21. The immunogenic composition according to any of paragraphs 1 to 7, comprising 20 μg/ml PE-PilA fusion protein; 20 μg/ml Protein D polypeptide; 6.6 μg/ml UspA2 polypeptide; 0.5 to 1.5 mg/ml, e.g.
1.2 mg/ml antioxidant (optionally L-methionine); 0.2 to 0.6 mg/ml e.g. 0.4 mg/ml poloxamer (optionally poloxamer 188); and 20 to 60 mg/ml, e.g. 40 mg/ml sucrose.
22. The immunogenic composition according to paragraph 20 or paragraph 21, further comprising polysorbate 80 (e.g. less than 0.03% (w/v) polysorbate 80).
23. A process for preparing an immunogenic composition according to paragraphs 1 to 22 comprising combining (i) Protein E from Haemophilus influenzae or an immunogenic fragment thereof and PilA from Haemophilus influenzae or an immunogenic fragment thereof, optionally as a fusion protein (optionally a PE-PilA fusion protein); a Protein D polypeptide; and an UspA2 polypeptide; with (ii) an anti-oxidant (optionally L-methionine) and (iii) poloxamer (optionally poloxamer 188).
24. A process for preparing an immunogenic composition according to paragraph 23 further comprising the step of freeze-drying the immunogenic composition.
25. A process for preparing an immunogenic composition according to paragraph 24 wherein the freeze-dried composition is suitable for reconstitution in an aqueous solution (optionally comprising an adjuvant, e.g. AS01E) prior to administration.
26. A kit comprising (i) a first container (optionally a vial) comprising an immunogenic composition according to any of paragraphs 1 to 15, optionally in solid form (optionally freeze-dried) and (ii) a second container (optionally a vial) comprising an adjuvant, optionally AS01E.
27. A vaccine comprising the immunogenic composition according to any of paragraphs 1-22.
28. The immunogenic composition according to any of paragraphs 1-22, or the kit according to paragraph 26 for use in the treatment or prevention of a disease caused by H. influenzae and/or M. catarrhalis.
29. The immunogenic composition according to any of paragraphs 1-22, or the kit according to paragraph 26 for use in the treatment or prevention of an acute exacerbation of COPD (AECOPD) in a subject, e.g. human.
30. The use of an immunogenic composition according to any of paragraphs 1-18, or the kit according to paragraph 26 in the manufacture of a medicament for the treatment or prevention of a disease caused by H. influenzae and/or M. catarrhalis.
31. The use of an immunogenic composition according to any of paragraphs 1-18, in the manufacture of a medicament for the treatment or prevention of an acute exacerbation of COPD (AECOPD) in a subject, e.g. human.
32. A method of treatment or prevention of a disease caused by H. influenzae and/or M. catarrhalis, in a subject, e.g. human, at risk, said method comprising administering to said subject an effective amount of an immunogenic composition according to any of paragraphs 1 to 18.
33. A method of treatment or prevention of acute exacerbations of an acute exacerbation of COPD (AECOPD) in a subject, e.g. human, at risk of developing an acute exacerbation of an acute exacerbation of COPD (AECOPD), said method comprising administering to said subject an effective amount of an immunogenic composition according to any of paragraphs 1 to 18.

In order that this invention may be better understood, the following examples are set forth. These examples are for purposes of illustration only and are not to be construed as limiting the scope of the invention in any manner.

### Examples

### Example 1: Formulation

TABLE 1
Immunogenic Composition

| | Concentration | | Concentration after | | Amount/ | |
|---|---|---|---|---|---|---|
| Antigen/excipient | (Final Bulk) | | reconstitution (625 μL) | | dose | |
| Protein D | 25 | μg/ml | 20 | μg/ml | 10 | μg |
| PE-PilA | 25 | μg/ml | 20 | μg/ml | 10 | μg |
| UspA2 | 8.3 | μg/ml | 6.6 | μg/ml | 3.3 | μg |
| Sucrose | 5% (w/v) | | 4% (w/v) | | 20 | mg |
| Poloxamer 188 | 0.05% (w/v) | | 0.04% (w/v) | | 0.2 | mg |
| KH₂PO₄/ | 12.5 | mM | 10 | mM | | |
| K₂HPO₄ | | | | | | |
| L-Methionine | 10 | mM | 8 | mM | 0.6 | mg |

| | | | | | |
|---|---|---|---|---|---|
| NaCl (residual) | ~5 | mM | ~4 | mM | — |
| Arginine | ~3 | mM | ~2 | mM | — |

| | | | |
|---|---|---|---|
| (residual) | | | |
| Water for injection | — | — | — |
| pH | 7.4 (formulation) | 7.0 (NaCl) 6.6(AS01E-3) | — |

Protein D (SEQ ID NO: 2) may be prepared as described in EP0594610.
PE-PilA (LVL735, SEQ ID NO: 9) may be prepared as described in WO2012/139225A1.
UspA2 (MC009, SEQ ID NO: 19) may be prepared as described in WO2015/125118A1.

Formulation of the immunogenic composition (Final Bulk) described in Table 1, was carried out at room temperature in glass bioreactors. Water for injection was mixed with a 15.75% w/v sucrose solution, a 100 mM KH₂PO₄/K₂HPO₄ pH 7.4 buffer (when diluted 8-fold), a 100 mM LMethionine solution and a 10% w/v Poloxamer 188 solution to reach the target concentrations of respectively: 5% sucrose, 12.5 mM of phosphate buffer, 10 mM L-Methionine and 0.05% w/v Poloxamer 188. The constituents were added using a peristaltic pump. For Poloxamer 188, a lower speed was applied to avoid foam formation. Subsequently the whole was mixed 15 minutes with a Ferrari impeller. Afterwards, under continuous mixing, the three antigens (Protein D, PE-PilA & UspA2) were added, to reach a concentration of 25 μg/ml of Protein D, 25 μg/ml of PE-PilA and 8.3 μg/ml of UspA2. When the last container of UspA2 was added, the mixture was homogenized for 15 minutes. This homogenization was followed by a pH check. For the two 1 L bioreactors (filled at 700 mL), the entire drug product volume was filtered using an OptiScale® 47 filter (Durapore® PVDF membrane 17.7 cm²—Polypropylene cartridge). The filtration was carried out with a peristaltic pump. Stored in static conditions at +2/+8° C. in stainless steel tank containers.

Before filling of the three COPD Drug Product repro batches, the formulations were sterile filtered using two OptiScale® 47 filters in series (0.22 μm, Durapore® PVDF membrane 17.7 cm²-Polypropylene cartridge). The filtration was carried out with a peristaltic pump. The three formulations were collected in Duran Schott glass containers. Immediately following the sterile filtration, the three drug product were transferred to the filling. Before starting the filling operations, the three batches were homogenized with a magnetic bar for 15 minutes. The agitation speed was defined to have a slight vortex. The filling was performed with a rotary pump without recirculation and without agitation. 0.5 mL (0.48-0.52 mL) of the formulated bulk was filled in 3 mL siliconized glass vials, with a needle of 1.4 mm of internal diameter, and then partially stoppered with siliconized stoppers.

The filled vials were arranged on bottomless trays and loaded on the selected freeze-dryers. The freeze-dryers used were the Martin Christ and the Martin Christ Epsilon. The vials were loaded on shelves precooled at −52° C. Then, these were freeze-dried using a 48h freeze-drying cycle.

### Example 2—Antioxidants for a Composition Containing Protein D. PE-PilA and UspA2

The sensitivity of the antigens present in a composition containing Protein D, PE-PilA and UspA2 to oxidation by VHP was assessed. It was demonstrated in the following experiments that methionine in Protein D is sensitive to oxidation, and in Protein D Methionine 192 is especially sensitive. A first experiment consisted of spiking with liquid H₂O₂ at a range of concentrations: 0, 150, 800, 1300 and 5000 ng/mL. The vaccine batch which was not spiked with H₂O₂ (0 ng/mL) corresponded to the reference, to generate non-stressed, non-oxidized reference samples. Samples spiked at 150 and 1300 ng/mL were representative of the exposure for manufacturing at 0.1 and 1 ppm v/v VHP in the isolator, respectively. The samples generated were then freeze dried and submitted to an accelerated stability plan at 25° C., 37° C. and 45° C. and a real time stability at 4° C.

The impact of the H₂O₂ spiking was assessed by performing analytical tests after the different accelerated stabilities. Protein D was found to be the most sensitive antigen to oxidation, demonstrated by mass spectrometry. We observed high percentages of oxidized methionines and a molecular weight shift was observed by SDS page and in RP-HPLC chromatograms. A clear impact of the H₂O₂ level on the level of oxidized Met192 was observed; the higher the quantity of H₂O₂, the more Met192 was oxidized. Based on M192 oxidation, correlations could be established to determine the level of oxidation of the other methionines of Protein D, therefore M192 was used as a probe for oxidation. Furthermore, it was demonstrated that oxidation of M192 occurred even for an equivalent stress of 0.1 ppm v/v in manufacturing.

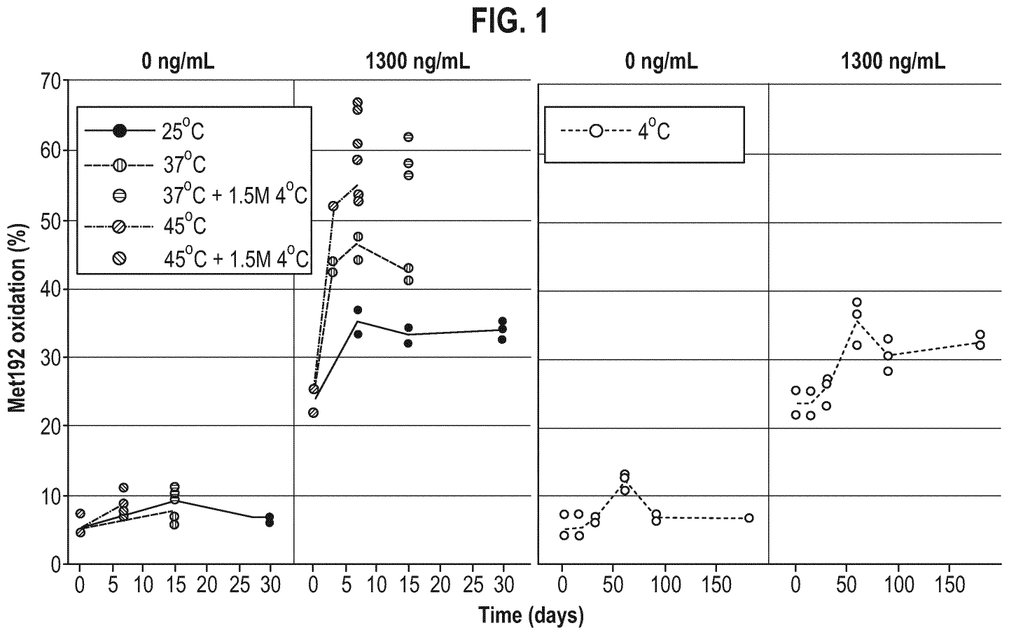

Results are shown in FIG. 1 to 3 as follows.

FIG. 1 shows mass spectrometry results for protein D Met192 oxidation over time for 0 and 1300 ng/mL H₂O₂ at different temperatures. +/−55% oxidation is reached after 7 days at 45° C.

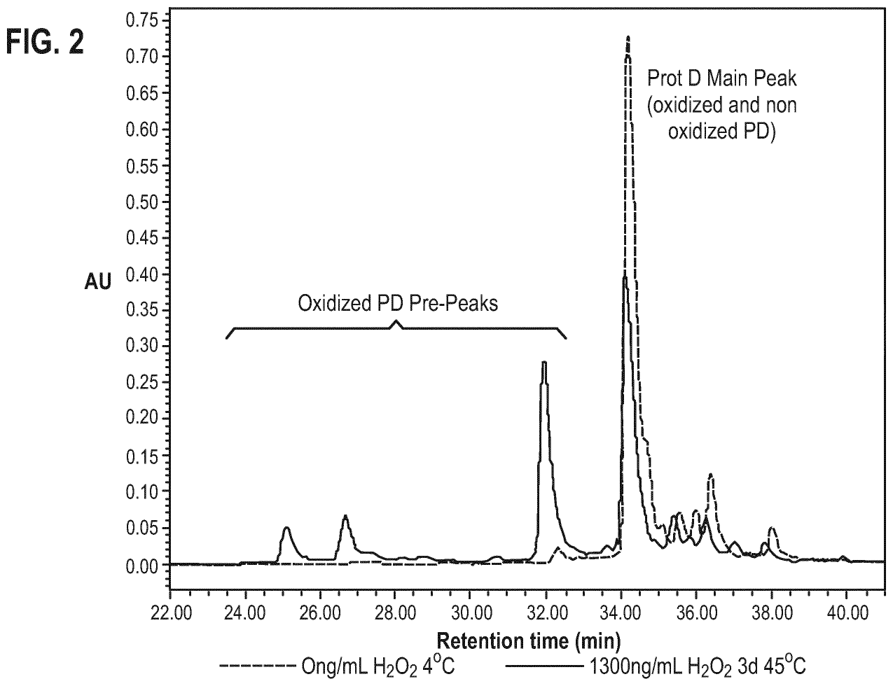

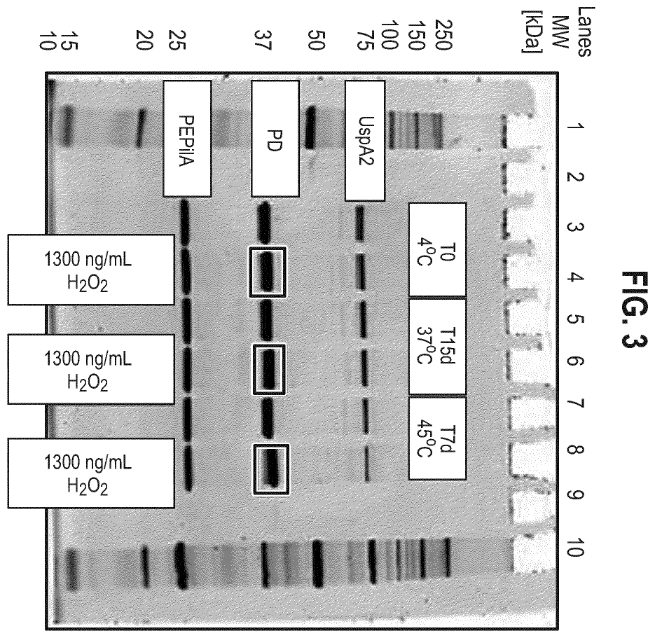

FIG. 2 shows a RP-HPLC chromatogram of oxidized protein D with 1300 ng/mL H₂O₂ stored for 3 days at 45° C. and of non-spiked protein D stored at 4° C.

FIG. 3 shows antigen profiles obtained by SDS-PAGE in non-reducing conditions of samples, oxidized or not, stored at 4° C., for 15 days at 37° C. and for 7 days at 45° C. Lanes 4, 6 and 8 show oxidative stress impact on the protein D profile.

### Assessment of Antioxidants

Experiments were designed to find out if the use of an antioxidant could prevent Protein D oxidation due to VHP oxidative stress encountered at manufacturing scale, and if so to determine which antioxidant would be most suitable. Once again, the trivalent vaccine was spiked (or not) with H₂O₂ and then freeze dried. Formulations with and without L-methionine or cysteine were tested. Formulations contained either L-methionine at 50 mM or cysteine at 30 mM, prior to freeze drying.

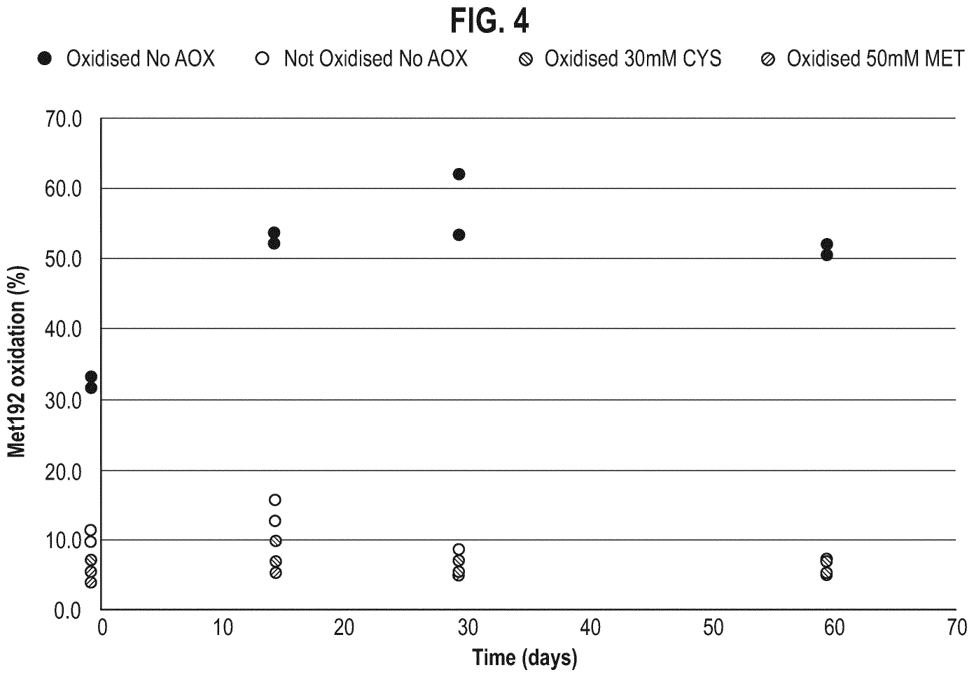

SDS-PAGE, hydrophobic variants RP-HPLC (which can also be referred to as purity by RP-HPLC) and Mass spectrometry were performed after 2 months at 37° C. on oxidized and non-oxidized samples containing either 50 mM methionine or 30 mM cysteine as antioxidant, or no antioxidant at all. Results are shown in FIGS. 4, 5 and 6.

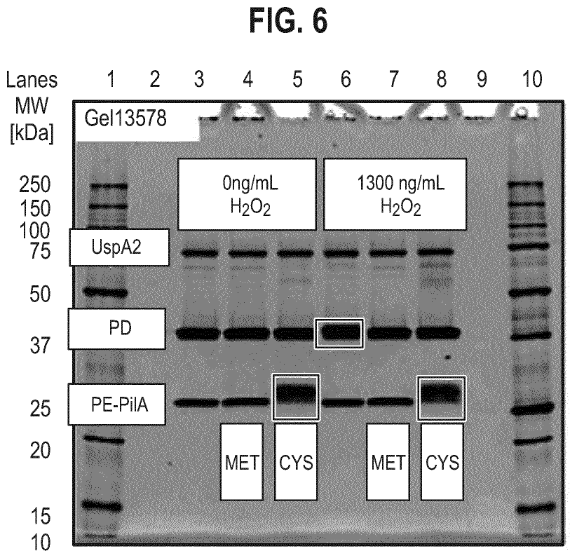

The antigen profiles obtained by SDS-PAGE in non-reducing conditions are shown in FIG. 6. Both cysteine and methionine prevented a molecular weight shift in protein D when samples were spiked with H₂O₂. Profile modifications of PE-PilA were observed in the presence of 30 mM cysteine. This was the case both for samples spiked with H₂O₂ and samples not spiked with H₂O₂. No profile modification was observed in the presence of methionine for the 3 antigens.

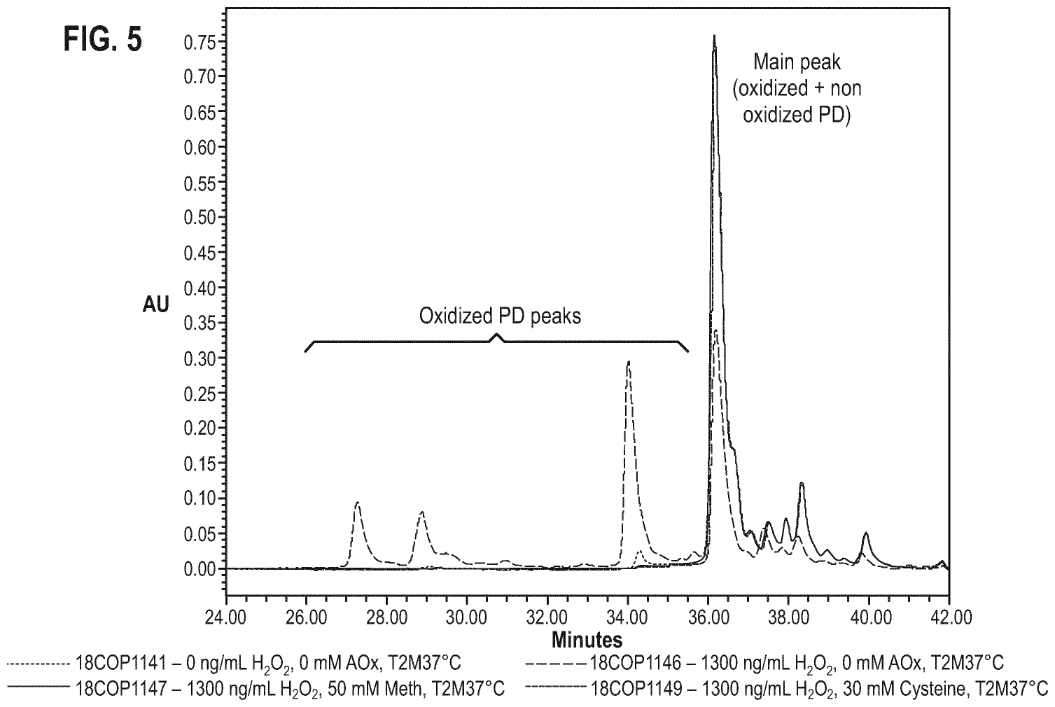

For the hydrophobic variants RP-HPLC, no profile modifications were observed in the presence of methionine for the 3 antigens compared to the non-oxidized reference sample. For cysteine no oxidation peaks were observed, though there was a decrease in Protein D main peak area, as for the H₂O₂ spiked control sample. The RP-HPLC chromatogram for protein D is shown in FIG. 5.

For the % methionine oxidation by mass spectrometry, antioxidant addition had a clear efficacy preventing oxidation for Protein D. The oxidation level in the presence of methionine was slightly lower than the oxidation level in presence of cysteine. No significant increase in oxidation was observed for PE-PilA or UspA2, in presence of H₂O₂, cysteine or methionine. The results for protein D only are shown in FIG. 4. Note that in FIG. 4 the 60 day results for samples with 50 mM methionine are not visible behind the dot representing 60 day results for samples with 30 mM cysteine.

Based on these results, methionine was identified as the most suitable antioxidant to protect against H₂O₂ mediated oxidation in this vaccine comprising Protein D, UspA2 and PE-PilA. Therefore, a methionine dose range experiment was performed to determine the exact methionine concentration that would be sufficient to prevent oxidation.

### Example 3—Dose Ranging Study to Determine Optimum Concentration of Methionine for Protection of Protein D Against Oxidation

This Example shows RP-HPLC and mass spectrometry data that were generated to define the optimal L-methionine concentration to avoid oxidation of Protein D.

The optimal concentration of L-methionine as an antioxidant was determined by spiking 1300 ng of H₂O₂ per mL into compositions containing Protein D, PE-PilA and UspA2, containing different concentrations of L-Met (Table 2 below). Subsequently the drug product was freeze dried and submitted to a stability plan (Table 3).

TABLE 2

| ID | Spiking [H₂O₂] | [MET] |
|---|---|---|
| Formulation | ng/mL | mM |
| 18COP1401 | 0 | 0 |
| 18COP1407 | 1300 | 0 |
| 18COP1402 | 1300 | 5 |
| 18COP1403 | 1300 | 10 |
| 18COP1404 | 1300 | 15 |
| 18COP1405 | 1300 | 25 |
| 18COP1406 | 1300 | 50 |

TABLE 3

| Time/ | T0 | T7 | T14 | T30 | T2 | T3 | T6 | T9 | T12 | T18 | T24 |
|---|---|---|---|---|---|---|---|---|---|---|---|
| Temp | days | days | days | days | months | months | months | months | months | Months | months |
| 4° C. | X | N/A | N/A | X | N/A | X | X | X | X | X | X |
| 37° C. | N/A | X | X | X | X | X | N/A | N/A | N/A | N/A | N/A |
| 45° C. | N/A | X | X | N/A | N/A | N/A | N/A | N/A | N/A | N/A | N/A |

The following tests were selected:
Hydrophobic variants by RP-HPLC:
   3 vials per condition/time point; run of 54 min (specific to protein D) was applied for all samples except for batches 18COP1401, 18COP1402 and 18COP1407 after 15 days at 45° C. for which a run of 154 min (for 3 antigens) was applied; samples were randomized in the sample set;
Methionine oxidation (Met192 of Protein D) by mass spectrometry: 6 vials for batch 18COP1401 (reference sample), 18COP1403 (oxidized sample with 10 mM Met) and 18COP1407 (oxidized reference sample) after 1 month at 37° C. The sample containing 10 mM L-Met was selected for mass spectrometry analysis based on the RP-HPLC data for all samples after 7 and 14 days at 37° C. and 45° C.

The key objective of this experiment was to select the optimal concentration for L-Met as antioxidant to protect the drug product from oxidation. The optimal concentration of methionine assures an oxidation level for H₂O₂ spiked samples that is at least as good as a non H₂O₂ spiked control sample.

To determine this range, the first step was to find the lowest L-Met concentration for which noninferiority compared to the control sample could be demonstrated. This was evaluated starting from the highest dose down to the lowest dose. The acceptance criteria to select this dose were based on a difference margin 6% by Mass Spectrometry (i.e. we looked for a deviation of no more than 6% of M192 oxidation from the reference, by mass spectrometry) or equivalent criteria in terms of oxidation peaks surface area for hydrophobic variants RP-HPLC.

Rather than measuring the methionine oxidation only directly by mass spectrometry, it was also estimated by RP-HPLC. It was found that the sum of RP-HPLC the oxidation peaks 1, 2 and 3 (see below) correlated well with the mass spectrometry measurements for M192 oxidation. Furthermore, the % area of peak 3 alone was found to be more than acceptable to correlate with mass spectrometry. The RP-HPLC method had the advantage of being faster and less variable at low oxidation values.

### Results and Discussion:

Hydrophobic variants by RP-HPLC RP-HPLC was used to look at purity.

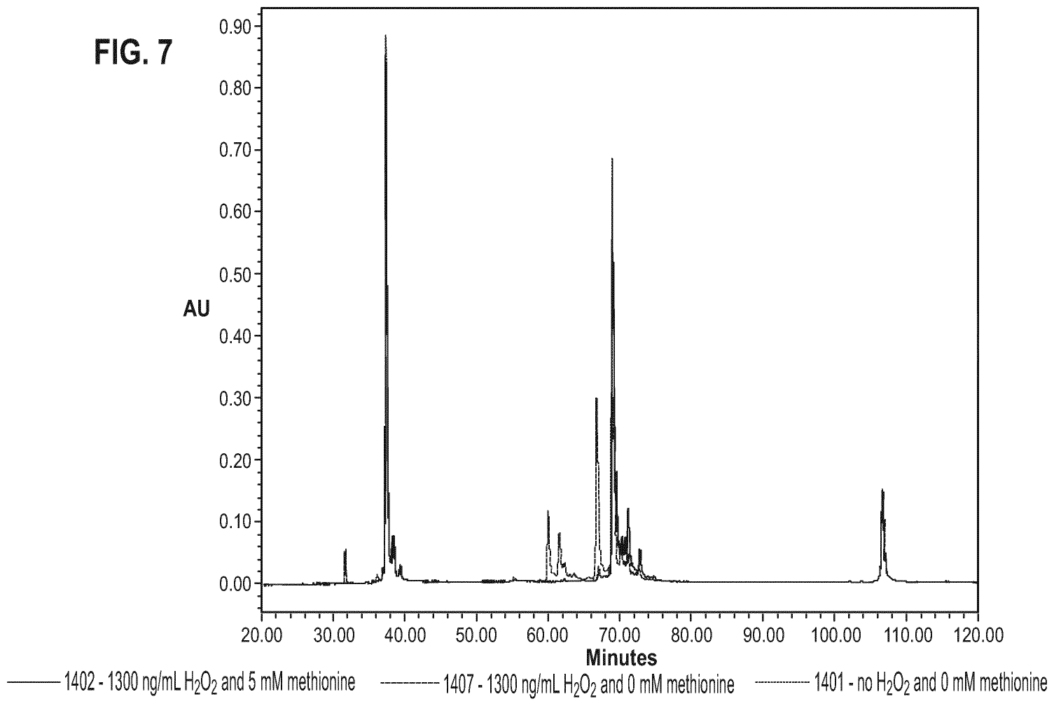

FIG. 7 shows hydrophobic variants HPLC 154 minutes chromatogram after 2 weeks 45° C. for samples 18COP1407 (0 mM L-Met+H₂O₂), 18COP1402 (5 mM L-Met+H₂O₂) and 18COP1401 (0 mM Met+no H₂O₂).

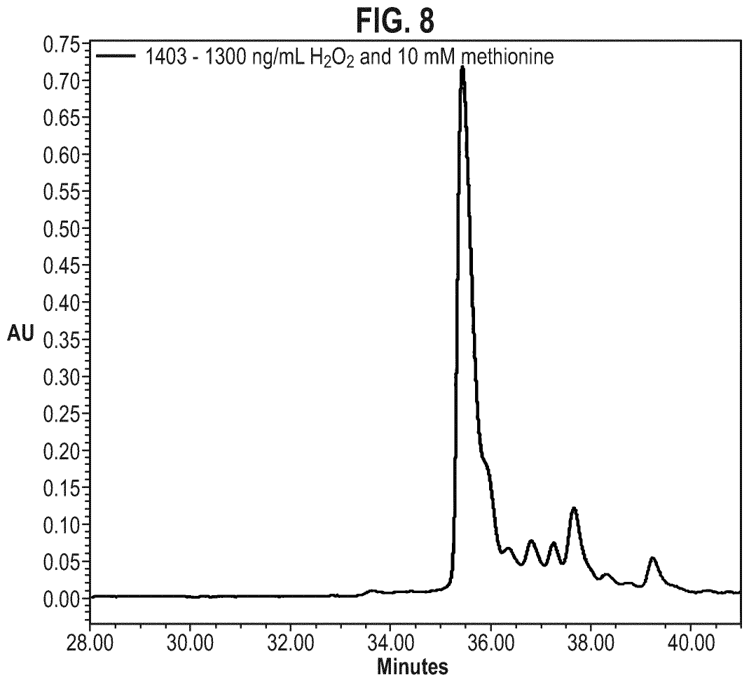

FIG. 8 shows hydrophobic variants HPLC minutes chromatogram after 2 weeks 45° C. for samples 18COP1403 (10 mM L-Met+H₂O₂).

FIG. 9 shows hydrophobic variants RP-HPLC % peak3, in the left panel not oxidized samples without antioxidant; in the right panel oxidized samples with methionine at different concentrations.

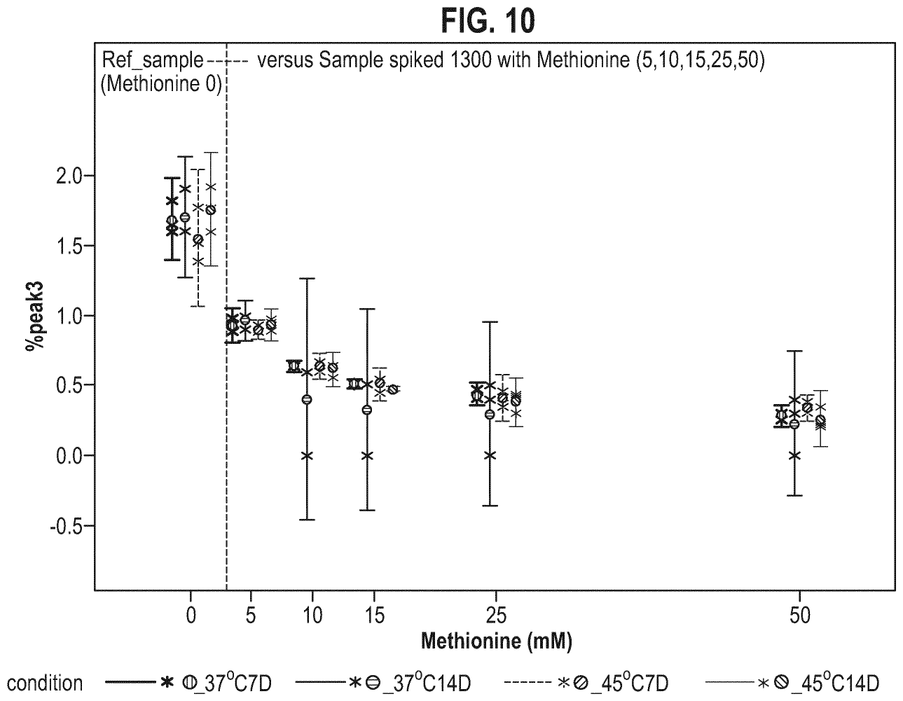

FIG. 10 shows hydrophobic variants RP-HPLC % peak3 oxidized samples with methionine at different concentrations.

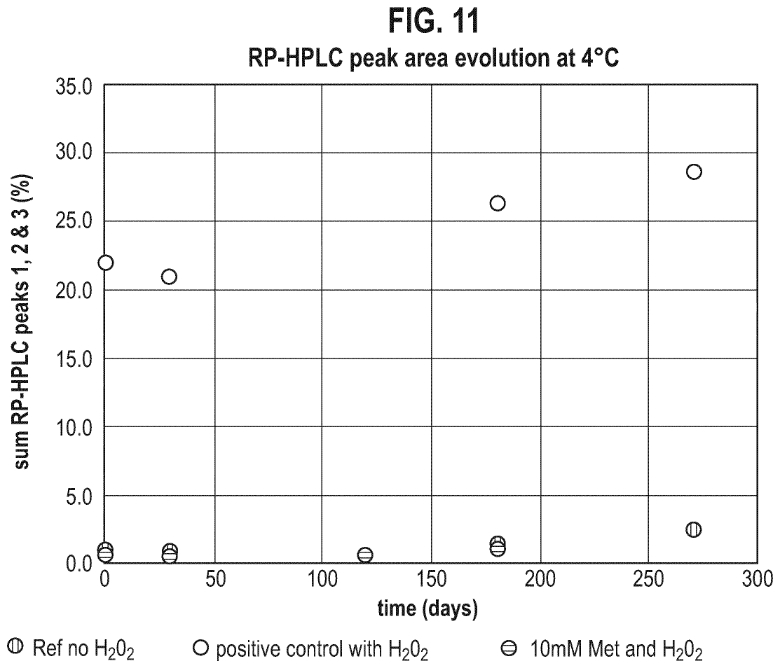

FIG. 11 shows the sum of area of peaks 1, 2 and 3 by RP-HPLC.

After 2 weeks at 45° C. no peaks were observed around 60-62 minutes for the sample containing 5 mM L-Met and H₂O₂ and for the reference sample containing no Methionine and no H₂O₂ (FIG. 7). After 67 minutes a slight oxidation peak was observed for both these samples. However, the peaks showed similar intensity. For the sample containing H₂O₂ but no methionine on the other hand, clear peaks were observed around 60, 62 and 67 minutes, named peaks 1, 2 and 3 respectively. Identical observations were made after 1 week 45° C. for the overlay with 10 mM of Methionine for which a chromatographic run focusing on protein D was performed.

No changes were observed in the profile of PE-PilA and UspA2 due to the presence of Methionine (FIG. 7). PE-PilA and UspA2 could be seen around 38 and 108 minutes respectively on the chromatogram. The small peak around 32 minutes for the sample containing H₂O₂ but no Methionine, was also observed during a PE-PilA analytical stress test exercise when PE-PilA was spiked with H₂O₂.

After 2 weeks at 45° C., for the sample containing H₂O₂ and 10 mM Methionine, no oxidation peaks were observed before the main protein D peak (FIG. 8), as was the case for the sample containing H₂O₂ and 5 mM Methionine and (FIG. 7). The overlay of the samples containing H₂O₂ and 5, 10 and 15 mM of Methionine after 1 week at 45° C. superimpose well and no meaningful oxidation peaks were observed before the main protein D peak for any of these samples (not shown).

The hydrophobic variants RP-HPLC % peak3 area is peak 3 area expressed as a percentage of the area of all the peaks together. % peak3 area showed a clear increase from around 2% for non-spiked reference samples (0 mM Met) up to around 27% for samples with no Methionine and spiked with 1300 ng of H₂O₂ per mL (see FIG. 9). For samples containing 5 mM of Methionine or more that were spiked with H₂O₂, no such increase in the hydrophobic variants RP-HPLC % peak3 area was observed. The evolution of the RP-HPLC % peak3 area between 0 and 5 mM L-Methionine was unknown, though it was noted that the increase of % peak3 had to have been sharp at some point since around 27% was observed for samples spiked with H₂O₂ containing no methionine.

Moreover, it was observed that the % peak3 area for samples with methionine and H₂O₂ was lower than for the reference sample containing no methionine and no spiked H₂O₂ (see FIG. 10). It was hypothesised this was due to some slight oxidation of the reference sample during the formulation, filling and freeze-drying processes while no methionine was present in the formulation to protect from this oxidation. Samples containing methionine (and spiked with H₂O₂) were protected from oxidation during this processing due to the presence of methionine. This could explain why a lower % peak3 area was observed for samples spiked with H₂O₂ and containing Methionine compared to the non-spiked no methionine reference sample.

Hereafter a summary of the statistical analysis is given that was performed on the Peak 3 area. Peak 3 was found more suitable for analysis than peak 2, as the observed signal for peak 2 was weak. In samples spiked with 1300 ng H₂O₂/mL, Peak 3 was observed at Day 7 and 14, 37° C. or 45° C. For samples which contained at least 5 mM of Methionine results for Area Peak 3 reached the noninferiority criteria, since the upper limit of the 2-sided standardized asymptotic 90% Cl for the group difference [treated minus control] was below 387000 and 260000 respectively [limit for noninferiority]). This corresponded to an acceptable difference of 9% and 6% respectively measured by Mass Spectrometry. The non-inferiority criteria were not met for samples spiked with 1300 ng H₂O₂/mL in the absence of methionine.

### Methionine Oxidation by Liquid Chromatography Coupled Mass Spectrometry

### Protein D

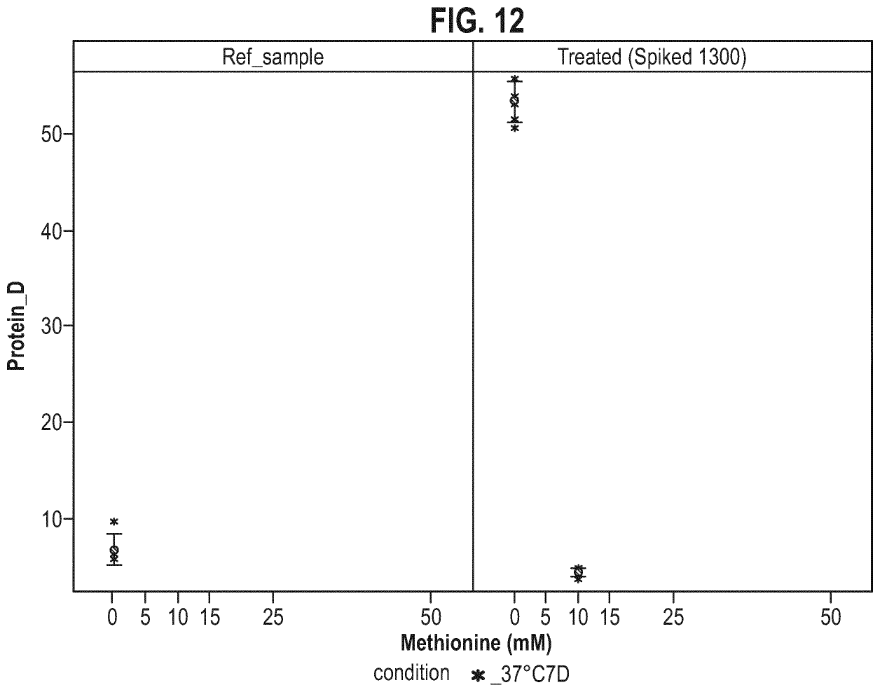

FIG. 12 shows liquid chromatography coupled mass spectrometry for protein D M192 oxidation in % after 1 month at 37° C. The left panel contains samples not spiked with H₂O₂, in the right panel samples received 1300 ng of H₂O₂ per mL before freeze drying. The error bars indicate the 95% confidence intervals.

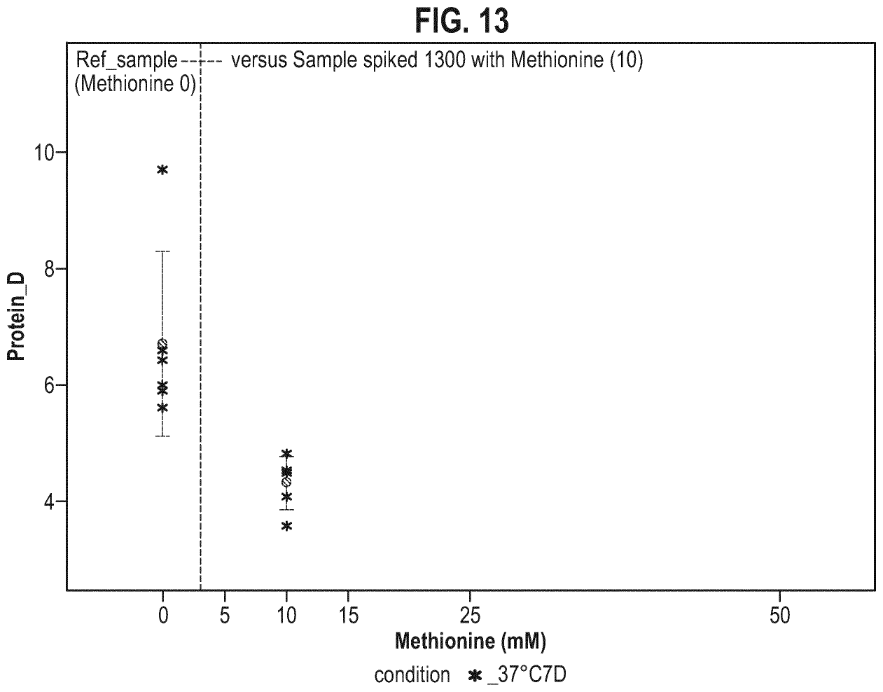

FIG. 13 shows liquid chromatography coupled mass spectrometry for protein D M192 oxidation in % after 1 month at 37° C. The left panel contains samples not spiked with H₂O₂, in the right panel samples received 1300 ng of H₂O₂ per mL before freeze drying and contain 10 mM of Methionine. The error bars indicate the 95% confidence intervals.

Mass spectrometry data for protein D Methionine 192 (M192) are depicted in FIG. 12. The sample that was not spiked with H₂O₂ and contained no Methionine showed very limited levels of M192 oxidation, whereas the sample spiked with H₂O₂ and containing no Methionine, clearly showed a high level of M192 oxidation—around 50%, and did not meet the statistical noninferiority criterion. The sample containing 10 mM of L-Met and spiked with H₂O₂ had an oxidation level lower or equal to the non-spiked reference. This sample met the statistical non-inferiority criterion, since the upper limit of the 2-sided standardized asymptotic 90% Cl for the group difference [treated minus control] was below 6% [limit for non-inferiority]. As for the hydrophobic variants RP-HPLC, the oxidation seemed slightly less for samples containing methionine compared to the non-spiked non-methionine containing samples (FIG. 13). A possible explanation for this observation is given above in the discussion of the RP-HPLC results.

### PE-PilA

For PE-PilA M215 oxidation, the levels of oxidation observed after 30 days at 37° C. were in the same range for all the tested samples (data not shown). No difference between the non H₂O₂ spiked reference and the H₂O₂ spiked sample containing 10 mM Methionine could be found.

### UspA2

For UspA2 M530 oxidation, the sample that was not spiked with H₂O₂ and contained no Methionine showed very limited levels of M530 oxidation (around 2%). The sample spiked with H₂O₂ and containing no Methionine, clearly showed a higher level of M530 oxidation; around 8% and did meet the statistical non-inferiority criterion. The sample containing 10 mM of L-Met and spiked with H₂O₂ had an oxidation level lower than the non-spiked reference (data not shown).

### Molar Considerations

Since oxidation is a chemical reaction it is interesting to express the quantities of oxidants and antioxidants in moles to get an idea of the molar ratios.

Molar wise the quantities of reactant and reagent are as shown in Table 4.

TABLE 4

| Quantity | Molar Quantity |
|---|---|
| 1300 ng/mL H₂O₂ spiked | 0.038 mM |
| Protein D concentration (25 μg/mL in drug | 0.0006 mM |
| product, 40 kDa per Protein D molecule) | |

It can be seen there is a 63-fold surplus of H₂O₂ molecules compared to Protein D. However, if 10 mM of Methionine is added to the drug product, there are 263 molecules of Methionine for each molecule of H₂O₂ spiked at 1300 ng/mL. Therefore, the addition of methionine greatly decreases the chances of H₂O₂ reacting with protein D.

### Conclusions

We showed that oxidation of protein D was observed for an equivalent manufacturing process executed at 0.1 ppm v/v or 1 ppm v/v H₂O₂ exposure in the gas phase. We demonstrated the addition of an antioxidant, specifically L-Methionine or cysteine, could prevent such oxidation.

The following points were taken into consideration when deciding on the Methionine concentration to be added to the drug product;
[Met] should protect for a 1 ppm v/v H₂O₂ process in the isolator to ensure manufacturing flexibility
10 mM of Met gives a sufficient safety margin and a data point at a lower concentration (5 mM) for which the RP-HPLC peak 3 area remains below the non-oxidized reference (no H₂O₂ spiked)
10 mM of Methionine has demonstrated good protection from oxidation based on mass spectrometry results for sensitive methionines on the 3 antigens present in the composition containing Protein D, PE-PilA and UspA2.
For these reasons a concentration of 10 mM L-Met was selected in this example for this vaccine.

### Example 4: Replacement of Polysorbate 80 by Poloxamer 188 as the Surfactant in the Drug Product

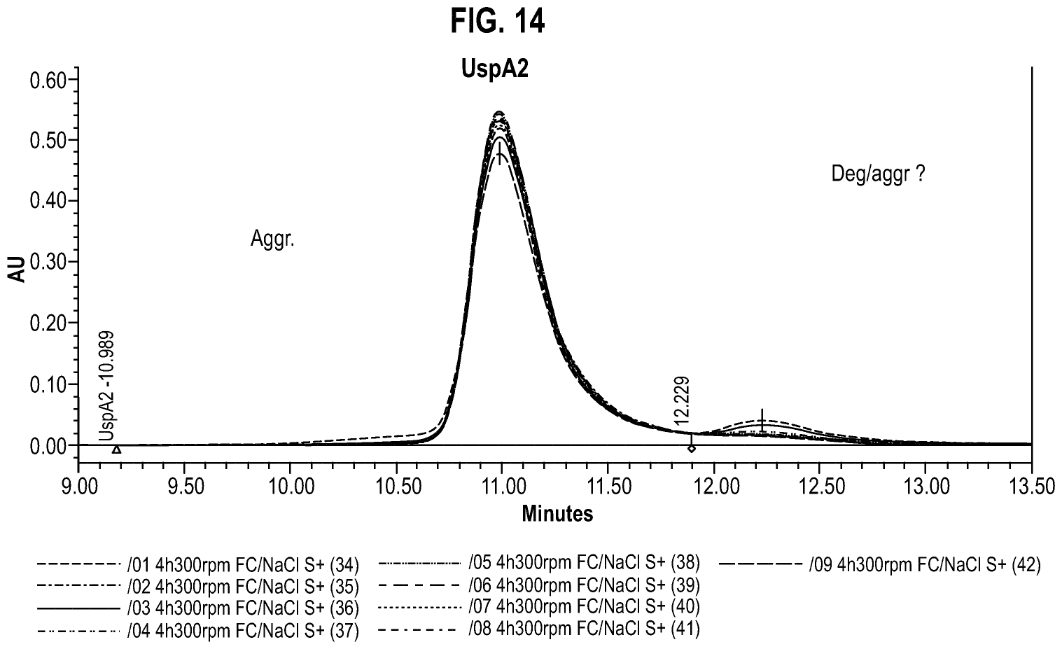

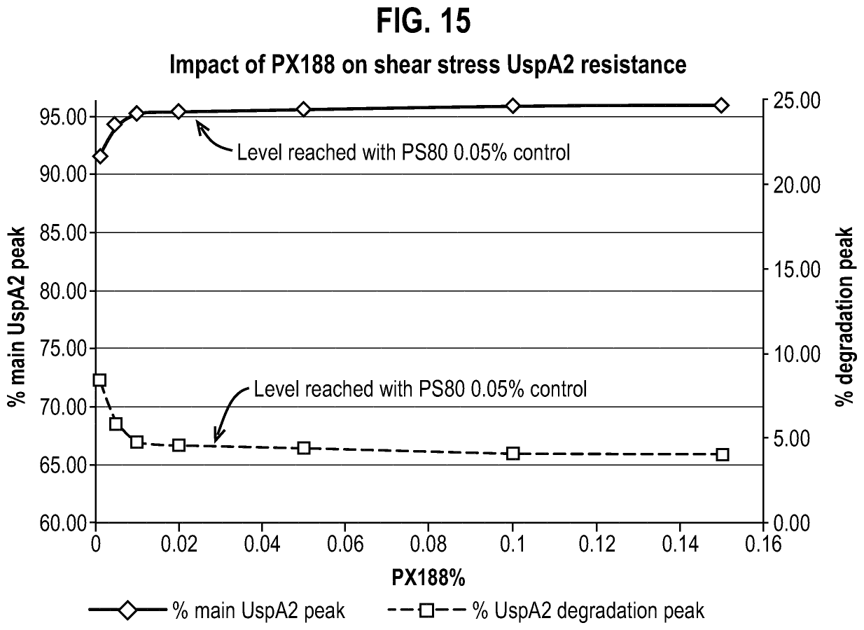

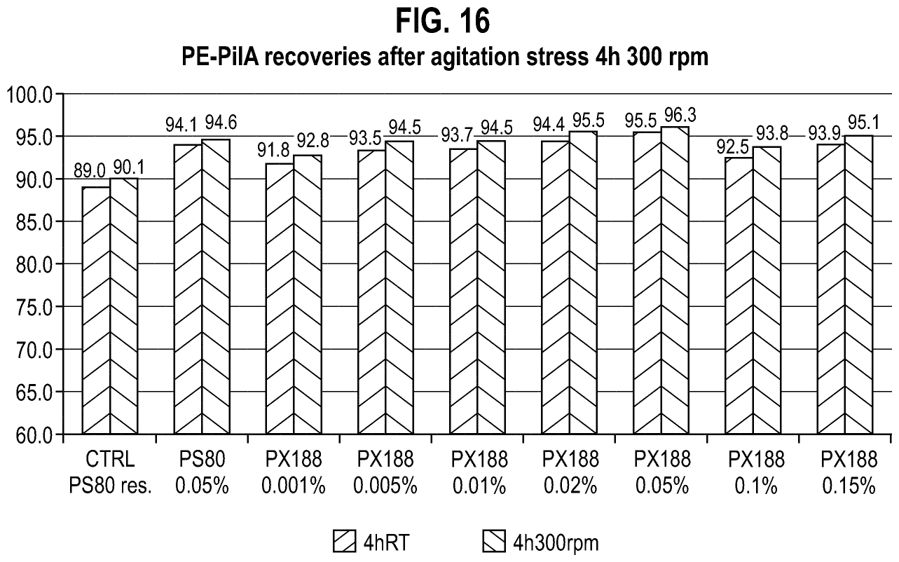

The surfactant polysorbate 80 (PS80) was replaced by poloxamer 188 (PX188) in the final formulation of the protein antigens: PE-PilA, Protein D and UspA2 (drug product). Trivalent formulations (PE-PilA, Protein D, UspA2 antigens as described above in Example 1) at 3×75 μg/ml with PX188 concentrations from 0.001% up to 0.15% along with PS80 at 0.05% were prepared and stirred for 4h at RT (2.3 mL in non-siliconized 3 mL vials). The formulations were further freeze-dried and then analyzed after reconstitution with NaCl. Turbidity, content and aggregation levels were analyzed for all samples. No or very slight difference could be observed in turbidity and content by HPLC RP measurement. Based on HPLC SEC, however differences could be seen for the aggregation level of one protein. The protein UspA2, was impacted by the magnetic agitation (FIG. 14, Table 5 and FIG. 15). UspA2 aggregation (Aggr.) and degradation (Deg.) were observed for the samples containing low PX188 content, but as of 0.02% w/v the HPLC SEC profile was similar to the one of non-agitated formulation. All PX188 amounts tested (0.001% to 0.15%) provided a % main UspA2 peak above that observed with residual PS80 alone, i.e. 91.56% to 95.97% compared to 90.12%. All PX188 amounts tested (0.001% to 0.15%) provided a % UspA2 degradation peak below that observed with residual PS80 alone, i.e. 4.03% to 8.44% compared to 9.88%. The 0.05% PX188 concentration was selected as the values for the % main UspA2 peak and % UspA2 degradation peak were closest to that observed with 0.05% PS80. Aspecific adsorption was also noticed for PE-PilA when formulated without any additional surfactant. For formulations containing 0.02% w/v of poloxamer 188 or more, the aspecific adsorption was reduced to the minimum. (see FIG. 16).

TABLE 5
Impact of PX188 on UspA2 resistance to shear stress

| PX188 content | % main | % UspA2 |
|---|---|---|
| (% w/v) | UspA2 peak | degradation peak |
| Res. PS80 | 90.12 | 9.88 |
| PS80 0.05% | 95.56 | 4.44 |
| PX188 0.001% | 91.56 | 8.44 |
| PX188 0.005% | 94.22 | 5.78 |
| PX188 0.01% | 95.24 | 4.76 |
| PX188 0.02% | 95.42 | 4.58 |
| PX188 0.05% | 95.60 | 4.40 |
| PX188 0.1% | 95.94 | 4.06 |
| PX188 0.15% | 95.97 | 4.03 |

### Example 5: Addition of Residual Polysorbate 80 (PS80) to Formulation with Poloxamer 188

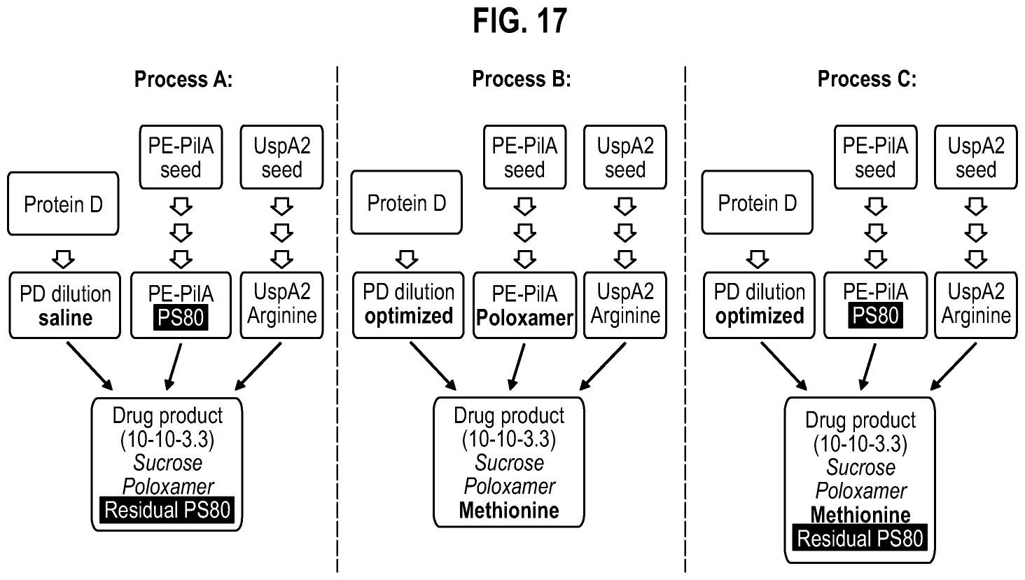

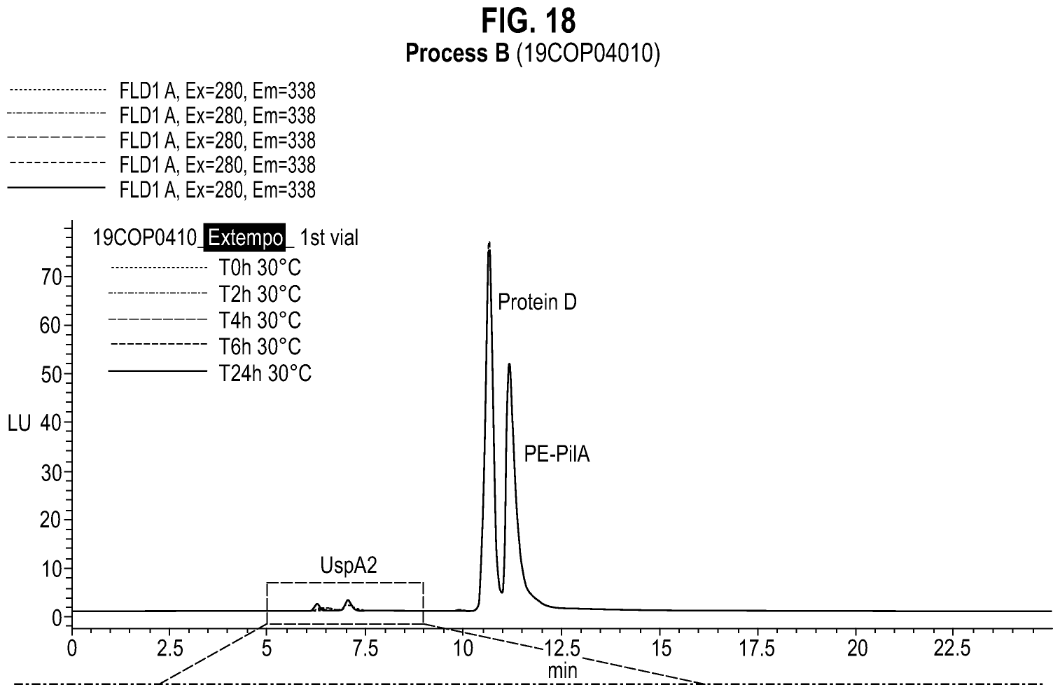

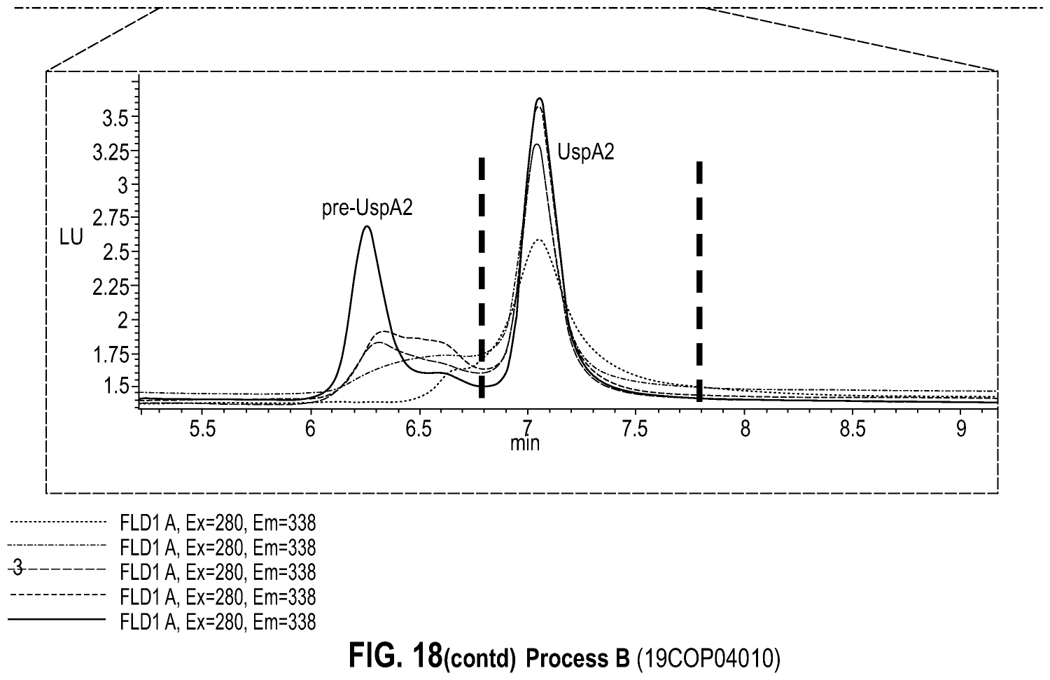

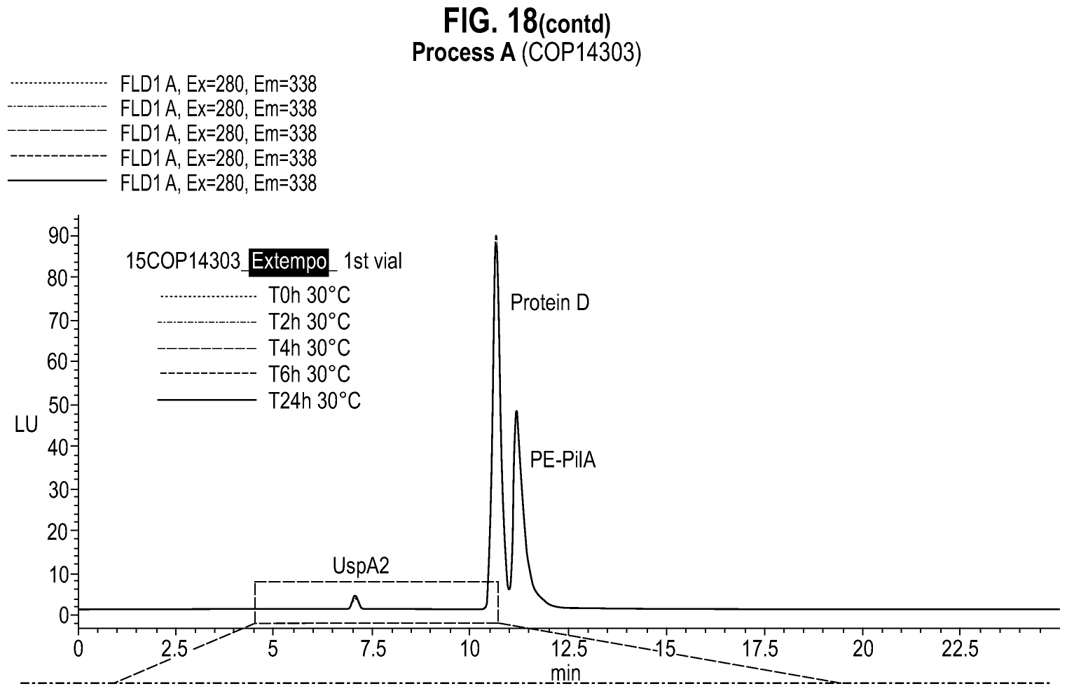

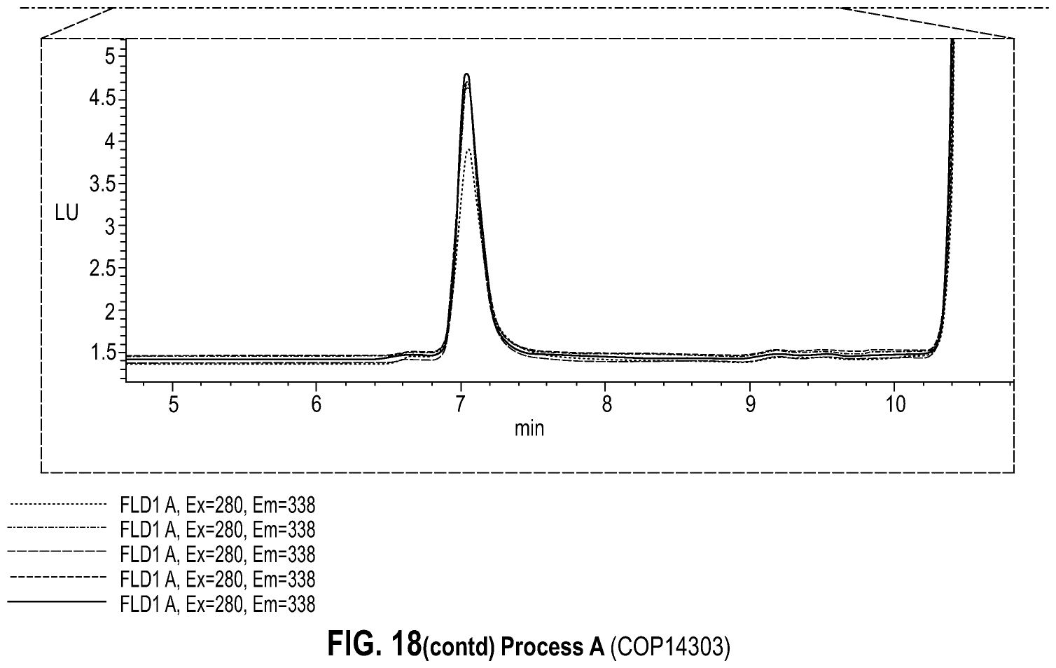

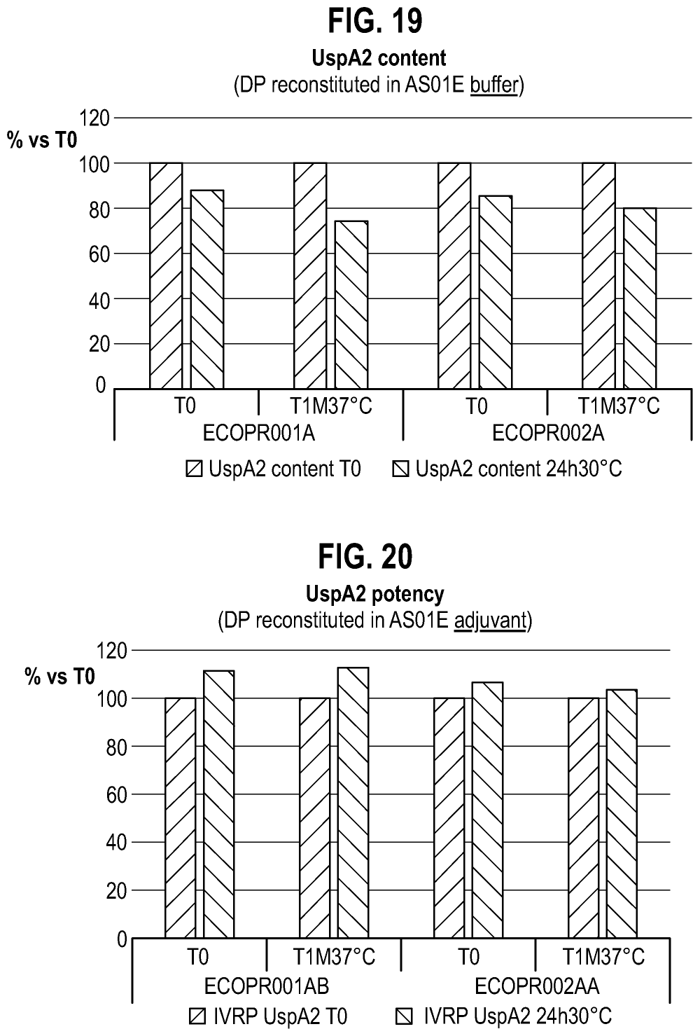

The stability of the reconstituted vaccine described in Example 4 above (using poloxamer 188 as the surfactant in the drug product) was investigated. The drug product prepared using PE-PilA drug substance which had been prepared according to Process B (where poloxamer 188 replaced PS80 in the preparation of PE-PilA drug substance as well as in the final formulation of the drug product, see FIG. 17) was compared to drug product prepared using PE-PilA drug substance which had been prepared according to Process A (where PS80 was used the preparation of PE-PilA drug substance and poloxamer 188 was used as the surfactant in the final formulation of the drug product). When Process B drug product was reconstituted with the adjuvant buffer (phosphate buffer saline pH 6.1), an increase in a pre-UspA2 peak by High Performance Size Exclusion Chromatography with Fluorescent detection (HPSEC-Fluo, also referred to as “SEC-HPLC-Fluo”) was observed over time (FIG. 18). Similarly, a decrease in UspA2 content by Reverse Phase-Ultra High Performance Chromatography (RP-UPLC) (FIG. 19) and UspA2 antigenic activity by Enzyme Linked Immunosorbent Assay (ELISA) (FIG. 20) was observed for Process B drug product in in-use conditions when the reconstitution was done with the adjuvant buffer.

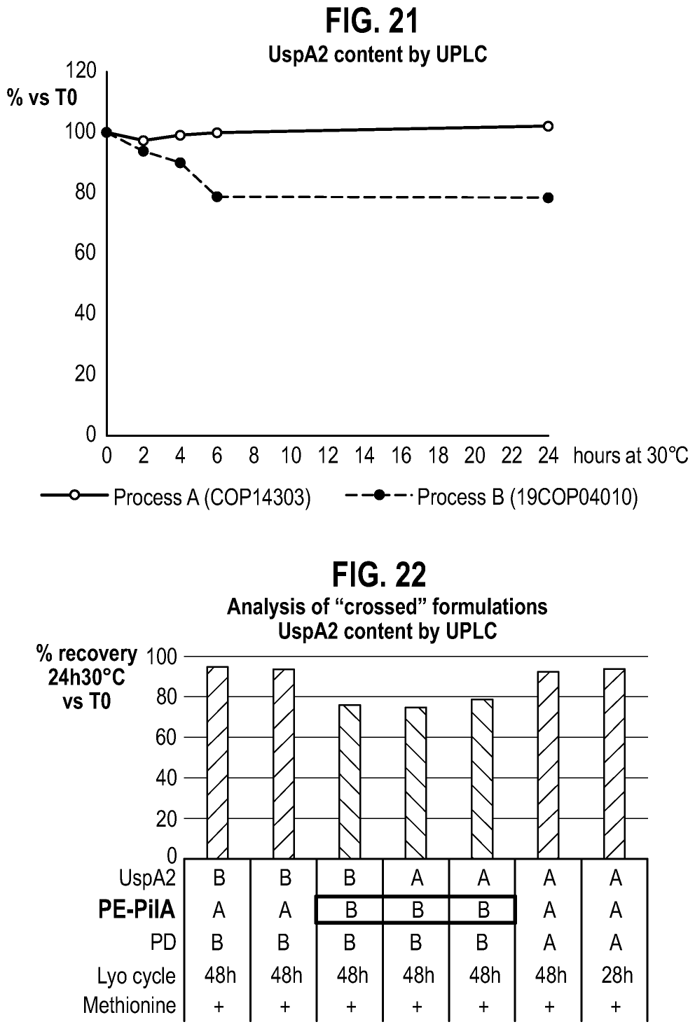

Several investigations were performed to understand the observations. A kinetics study was performed at +30° C. measuring UspA2 content by UPLC at timepoints between 0 and 24h following reconstitution with the adjuvant buffer, showing a difference of behaviour between Process A drug product and Process B drug product (FIG. 21). The Process A material was stable (decrease of UspA2 content and modification of HPSEC-FLuo profile was not observed).

Further investigations were performed with crossed formulations prepared with drug substances from different origins, and this confirmed that the difference of behaviour was mediated by the change of the process for preparing the PE-PilA drug substance from process A to process B (FIG. 22). The addition of L-Methionine was excluded as the cause of the effect because L-Methionine was present in all crossed formulations.

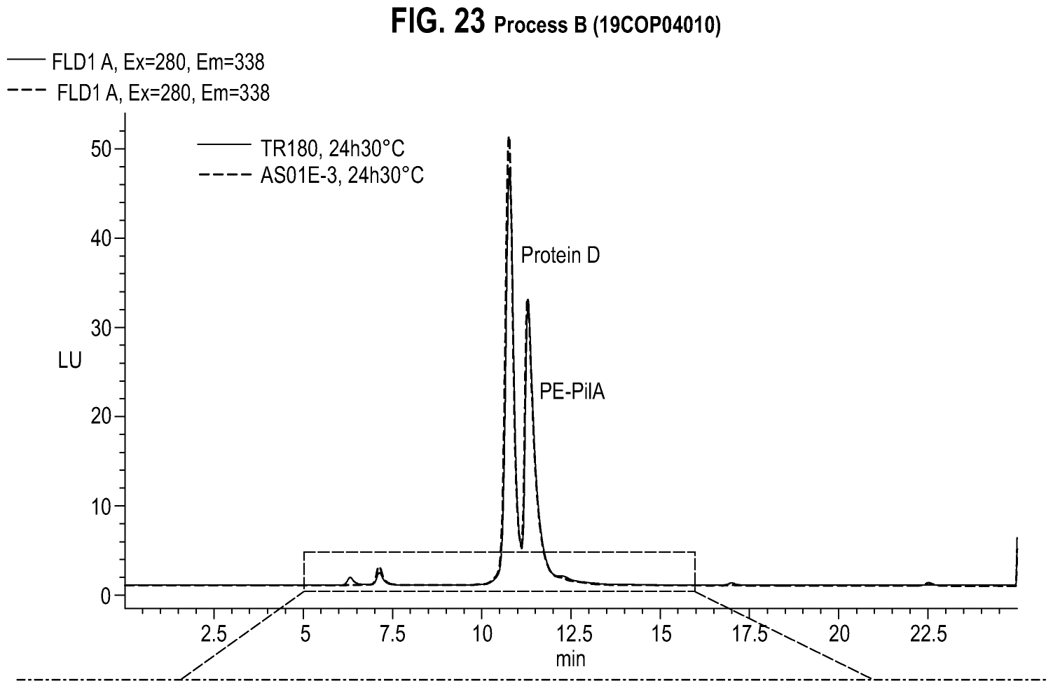

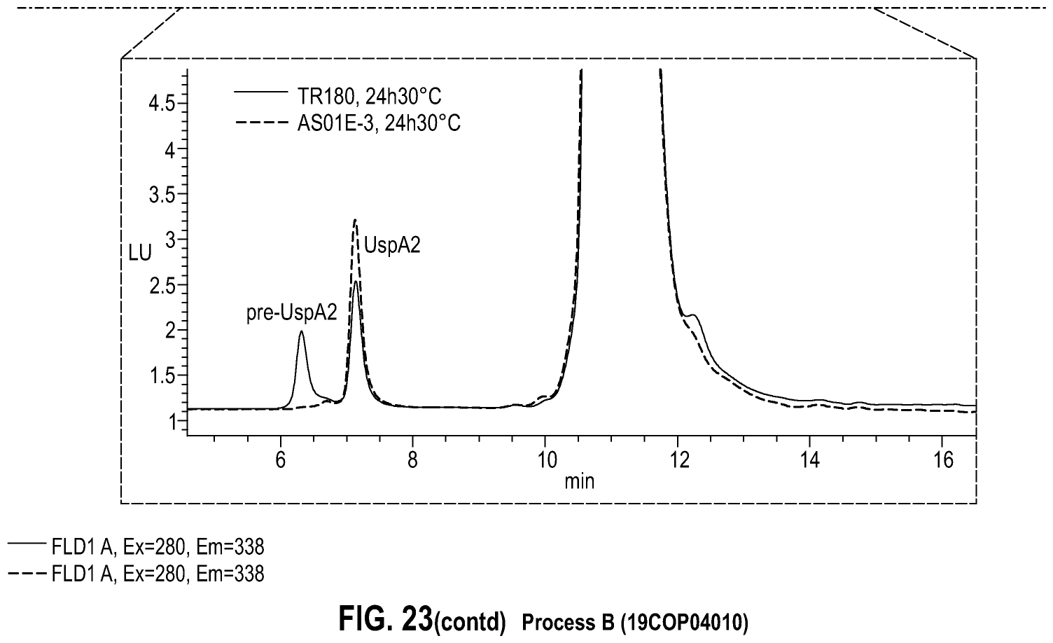

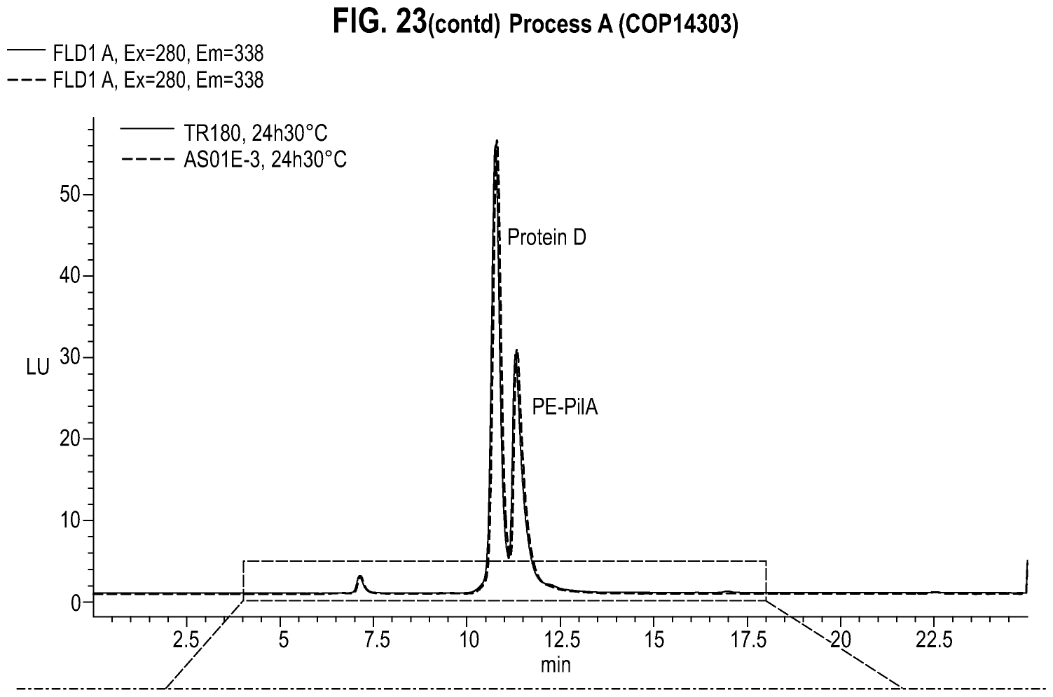

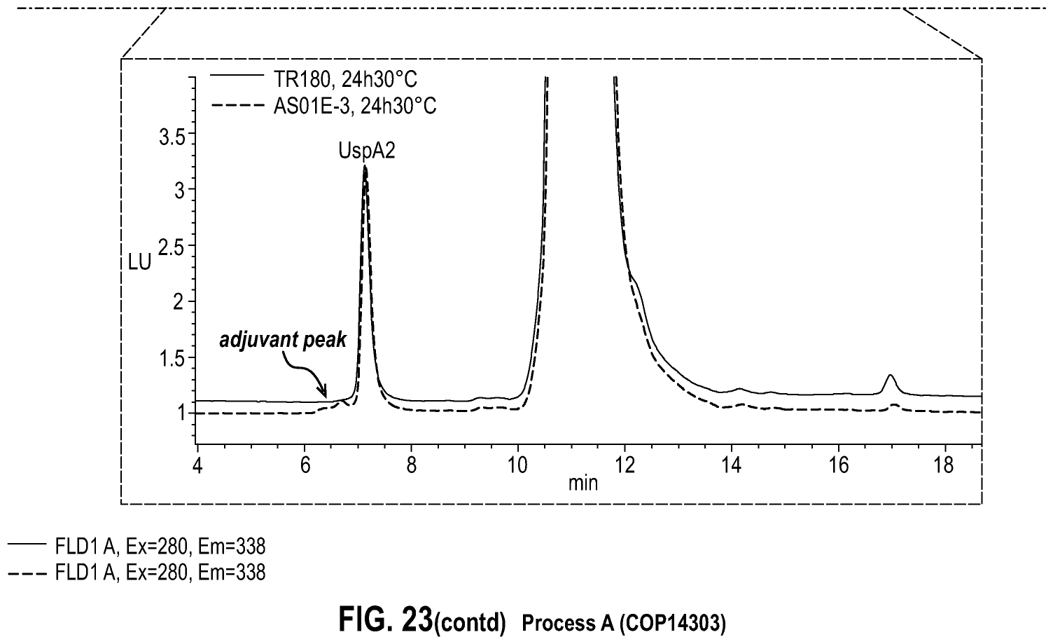

No modification of the profile by SEC-HPLC-Fluo was observed in in use conditions (24 hours+30° C.) when the drug product was reconstituted with the AS01E adjuvant (FIG. 23). No UspA2 pre-peak was observed when drug product was reconstituted with AS01E adjuvant.

When Process A PE-PilA drug substance was used, the residual amount of polysorbate 80 present in the drug product was estimated to be around 3 μg per dose (below the critical micellar concentration).

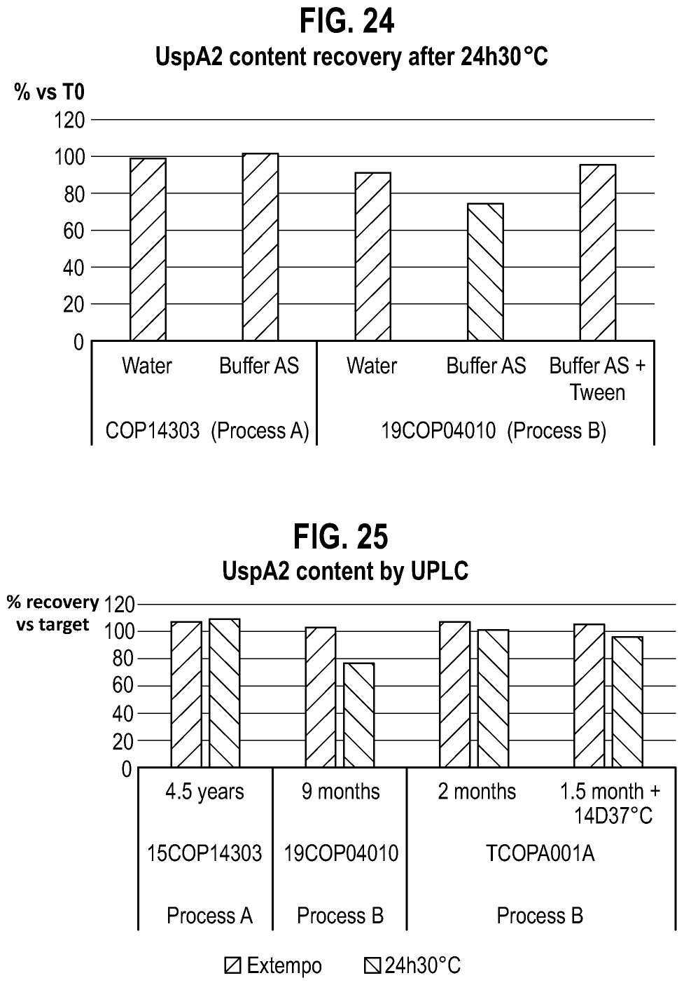

Investigations were also performed by reconstituting the drug product with the buffer of the adjuvant spiked with a residual amount of polysorbate 80 (6.66 μg/ml) equivalent to the amount present in Process A drug product. This was added directly after reconstitution by adding a concentrated solution of PS80. No UspA2 decrease by RP-UPLC was observed during in use conditions (24 hours+30° C.). It was also observed that when the drug product was reconstituted in water (conditions for the testing of the drug product as part of a release panel), and incubated 24 hours at +30° C., no decrease was observed with the Process A drug product, and a limited decrease (below 10%) for the Process B drug product, while the decrease with adjuvant buffer alone was stronger (−25%) (FIG. 24).

For Process B drug product, it was observed that the decrease of the UspA2 content was more pronounced (in use conditions in the buffer of the adjuvant) in older lots, while the decrease was more limited on recent lots 1.5 to 2 months (TCOPA001A), even after artificially aging (14 days+37° C.) (FIG. 25). UspA2 instability (drug product reconstituted in adjuvant buffer) appeared higher with older (or artificially aged) lots. A decrease of antigenic activity for older lots at 9 months (19COP04010) when they were reconstituted in adjuvant buffer and incubated 24h30° C.

In summary, UspA2 instability was not observed when PS80 was present:
when PS80 was spiked in the reconstituted vaccine
when PS80 was included in the formulation, or
when PE-PilA material was prepared using Polysorbate 80 (Process A)

These showed no loss of UspA2 content, no UspA2 pre-peak and a reduction in in vitro potency when the Drug Product was reconstituted in adjuvant.

The investigations performed confirmed that there was no antigen integrity issue when the drug product was reconstituted in the adjuvant (AS01E). However, this represents a risk for the long term stability of the Process B drug product, as the destabilization induced by the adjuvant buffer is more pronounced in older lots. Therefore, the following immunogenic composition is proposed in which residual Polysorbate 80 is present in the drug product formulation (Table 6):

TABLE 6
Immunogenic Composition

| Antigen/ | Concentration | | Concentration after | | Amount/ | |
|---|---|---|---|---|---|---|
| excipient | (Final Bulk) | | reconstitution (625 μL) | | dose | |
| Protein D | 25 | μg/ml | 20 | μg/ml | 10 | μg |
| PE-PilA | 25 | μg/ml | 20 | μg/ml | 10 | μg |
| UspA2 | 8.3 | μg/ml | 6.6 | μg/ml | 3.3 | μg |
| Sucrose | 5% (w/v) | | 4% (w/v) | | 20 | mg |
| Poloxamer 188 | 0.05% (w/v) | | 0.04% (w/v) | | 0.2 | mg |
| KH₂PO₄/ | 12.5 | mM | 10 | mM | | |
| K₂HPO₄ | | | | | | |
| L-Methionine | 10 | mM | 8 | mM | 0.6 | mg |
| NaCl (residual) | ~5 | mM | ~4 | mM | approximately | |
| | | | | | 0.1 | mg |
| | | | | | (4.5 mg when | |
| | | | | | reconstituted | |
| | | | | | with | |
| | | | | | adjuvant) | |
| Arginine | ~3 | mM | ~2 | mM | <0.2 | mg |

| (residual) | | | | |
|---|---|---|---|---|
| Polysorbate 80 | ~0.0008% (w/v) | ~0.00064% (w/v) | approximately | |
| (residual) | | | 3.2 | μg |

| | | | |
|---|---|---|---|
| Water for | — | — | — |
| injection | | | |
| pH | 7.4 | 7.0 (NaCl) | — |
| | (formulation) | 6.6(AS01E-3) | |

Protein D (SEQ ID NO: 2) may be prepared as described in EP0594610.

PE-PilA (LVL735, SEQ ID NO: 9) may be prepared as described in WO2012/139225A1.

UspA2 (MC009, SEQ ID NO: 19) may be prepared as described in WO2015/125118A1.

A new process (Process C, see FIG. 17) is proposed which will keep PS80 as surfactant in the PE-PilA drug substance rather than poloxamer 188. Note that the concentration of Poloxamer 188, which is added as an excipient in the drug product will not be changed and remains at 0.05% w/v. In Process C the PS80 surfactant is added at the end of the PE-PilA drug substance process, after ultrafiltration step and before bioburden control filtration. The PS80 concentration target in the PE-PilA drug substance is 0.04% w/v and 20% variation around this target is considered aligned with proposed specification range i.e. 0.032-0.048% w/v (to be confirmed according to the experiment described in Example 6 below). Variation of the polysorbate 80 and PE-PilA antigen concentration at the level of the PE-PilA drug substance results in a variation of the PS80 concentration at the level of the drug product of 0.00036 up to 0.00120% w/v. This will be further investigated according to the following Example.

### Example 6: Residual Polysorbate 80 (PS80) in Formulation with Poloxamer 188

The goal of this evaluation is to evaluate if changes in the residual polysorbate 80 (PS80) concentration could impact the aggregation profile (HPSEC-Fluo), content (UspA2 by RP-UPLC) and antigenic activity for UspA2 (ELISA).

Five different drug product batches will be produced with different levels of residual PS80 concentration and a fixed Poloxamer 188 concentration and subsequently filled at 0.5 mL and freeze dried:
1 group at the lower end (0.00036% w/v of PS80)
1 group at the higher end (0.00120% w/v of PS80)
1 group around the target (0.00079% w/v of PS80)
1 intermediate group (0.00065% w/v of PS80)
1 negative control group with no PS80 but only Poloxamer 188 at the level of the PE-PilA drug substance. This group is expected to confirm previously observed impact on the Critical Quality Attributes.

TABLE 7
residual polysorbate 80 in the Drug product in function of PE-PilA antigen
concentration and Polysorbate 80 concentration at the level of the Drug substance

| | Low | | | High | |
|---|---|---|---|---|---|
| | residual | Intermediate | | residual | PX 188 |
| | PS80 | low PS80 | Target PS80 | PS80 | control |
| Drug substance | | | | | |
| [PE-PilA] | 2233.0 | 1535.3 | 1269.7 | 1244.3 | 1497.7 |
| (μg/mL) | | | | | |
| PS80 (% w/v) | 0.032 | 0.040 | 0.040 | 0.048 | 0 |
| PX188 (% w/v) | 0 | 0 | 0 | 0 | 0.20 |
| Ratio PS80/Ag | 14.3 | 26.1 | 31.5 | 38.6 | 0 |
| *10\hat{ }6 | | | | | |
| Drug product | | | | | |
| [PE-PilA] | 25 | 25 | 25 | 25 | 25 |
| (μg/mL) | | | | | |
| PS80 (% w/V) | 0.00036 | 0.00065 | 0.00079 | 0.00120* | 0 |
| PX188 (% w/V) | 0.05 | 0.05 | 0.05 | 0.05 | 0.05 |
| Sample name | ECOPR012 | ECOPR013 | ECOPR014 | ECOPR015 | ECOPR016 |

**PS80 was added to the formulation in order to reach the residual value of 0.00120% w/V

The samples will be followed in real time (6 Months, 1, 2, 3, 4 and 5 years at 4° C.) and accelerated stability (1 M at 37° C.). After reconstitution of the freeze-dried samples with AS01_{E} buffer, the product will be held for 24h at 30° C. The purity by HPSEC-Fluo will also be tested at t0 to see the qualitative evolution of the profile over time after reconstitution. Analytical repeats will be performed on the accelerated stability samples; the real time stability samples will be tested n=1.

TABLE 8
List of analytical methods and performance

| CQA/PA | Test | Units | Method variability | Type |
|---|---|---|---|---|
| purity | HPSEC Fluo | Area % | 10.5% RSD for | Release |
| | | | UspA2 | |
| | | | 0.5% RSD for Protein | |
| | | | D | |
| | | | 1.0% RSD for PE-PilA | |
| Antigenic | ELISA for | % | +/− 6.7% (UspA2) | Release |
| activity | UspA2 | | repeatability | |
| Content | RP-UPLC | μg/mL | 2% | Release |
| | | | (repeatability) | |

Moreover the Tg′ of the different samples will be measured to assess if the introduction of PS80 as a residual could have an impact on the current freeze drying cycle.

SEQUENCES:

SEQ ID NO 1: Protein D (364 amino acids)
MetLysLeuLysThrLeuAlaLeuSerLeuLeuAlaAlaGlyValLeuAlaGly
CysSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
SEQ ID NO: 2: Protein D fragment with MDP tripeptide from NS1
(348 amino acids)
MetAspProSerSerHisSerSerAsnMetAlaAsnThrGlnMetLysSerAspLysIle
IleIleAlaHisArgGlyAlaSerGlyTyrLeuProGluHisThrLeuGluSerLysAla
LeuAlaPheAlaGlnGlnAlaAspTyrLeuGluGlnAspLeuAlaMetThrLysAspGly
ArgLeuValValIleHisAspHisPheLeuAspGlyLeuThrAspValAlaLysLysPhe
ProHisArgHisArgLysAspGlyArgTyrTyrValIleAspPheThrLeuLysGluIle
GlnSerLeuGluMetThrGluAsnPheGluThrLysAspGlyLysGlnAlaGlnValTyr
ProAsnArgPheProLeuTrpLysSerHisPheArgIleHisThrPheGluAspGluIle
GluPheIleGlnGlyLeuGluLysSerThrGlyLysLysValGlyIleTyrProGluIle
LysAlaProTrpPheHisHisGlnAsnGlyLysAspIleAlaAlaGluThrLeuLysVal
LeuLysLysTyrGlyTyrAspLysLysThrAspMetValTyrLeuGlnThrPheAspPhe
AsnGluLeuLysArgIleLysThrGluLeuLeuProGlnMetGlyMetAspLeuLysLeu
ValGlnLeuIleAlaTyrThrAspTrpLysGluThrGlnGluLysAspProLysGlyTyr
TrpValAsnTyrAsnTyrAspTrpMetPheLysProGlyAlaMetAlaGluValValLys
TyrAlaAspGlyValGlyProGlyTrpTyrMetLeuValAsnLysGluGluSerLysPro
AspAsnIleValTyrThrProLeuValLysGluLeuAlaGlnTyrAsnValGluValHis
ProTyrThrValArgLysAspAlaLeuProGluPhePheThrAspValAsnGlnMetTyr
AspAlaLeuLeuAsnLysSerGlyAlaThrGlyValPheThrAspPheProAspThrGly
ValGluPheLeuLysGlyIleLys
SEQ ID NO: 3:
SerSerHisSerSerAsnMetAlaAsnThr
SEQ ID NO: 4: Protein E from H. influenzae
MKKIILTLSL GLLTACSAQI QKAEQNDVKL APPTDVRSGY IRLVKNVNYY IDSESIWWDN
QEPQIVHFDA VVNLDKGLYV YPEPKRYARS VRQYKILNCA NYHLTQVRTD FYDEFWGQGL
RAAPKKQKKH TLSLTPDTTL YNAAQIICAN YGEAFSVDKK
SEQ ID NO: 5: Amino acids 20-160 of Protein E
I QKAEQNDVKL APPTDVRSGY IRLVKNVNYY IDSESIWWDN QEPQIVHFDA VVNLDKGLYV
YPEPKRYARS VRQYKILNCA NYHLTQVRTD FYDEFWGQGL RAAPKKQKKH TLSLTPDTTL
YNAAQIICAN YGEAFSVDKK
SEQ ID NO: 6 PilA from H. influenzae
MKLTTQQTLK KGFTLIELMI VIAIIAILAT IAIPSYQNYT KKAAVSELLQ ASAPYKADVE LCVYSTNETT
NCTGGKNGIA ADITTAKGYV KSVTTSNGAI TVKGDGTLAN MEYILQATGN AATGVTWTTT
CKGTDASLFP ANFCGSVTQ
SEQ ID NO: 7 Amino acids 40-149 of PilA from H. influenzae strain 86-028NP
T KKAAVSELLQ ASAPYKADVE LCVYSTNETT NCTGGKNGIA ADITTAKGYV KSVTTSNGAI
TVKGDGTLAN MEYILQATGN AATGVTWTTT CKGTDASLFP ANFCGSVTQ
SEQ ID NO: 8: LVL735 (protein): (pelB sp)(ProtE aa 20-160)(GG)(PilA aa40-149)
MKYLLPTAAA GLLLLAAQPA MAIQKAEQND VKLAPPTDVR SGYIRLVKNV NYYIDSESIW
VDNQEPQIVH FDAVVNLDKG LYVYPEPKRY ARSVRQYKIL NCANYHLTQV RTDFYDEFWG
QGLRAAPKKQ KKHTLSLTPD TTLYNAAQII CANYGEAFSV DKKGGTKKAA VSELLQASAP
YKADVELCVY STNETTNCTG GKNGIAADIT TAKGYVKSVT TSNGAITVKG DGTLANMEYI
LQATGNAATG VTWTTTCKGT DASLFPANFC GSVTQ
SEQ ID NO: 9: PE-PilA fusion protein without signal peptide
IQKAEQND VKLAPPTDVR SGYIRLVKNV NYYIDSESIW VDNQEPQIVH FDAVVNLDKG
LYVYPEPKRY ARSVRQYKIL NCANYHLTQV RTDFYDEFWG QGLRAAPKKQ KKHTLSLTPD
TTLYNAAQII CANYGEAFSV DKKGGTKKAA VSELLQASAP YKADVELCVY STNETTNCTG
GKNGIAADIT TAKGYVKSVT TSNGAITVKG DGTLANMEYI LQATGNAATG VTWTTTCKGT
DASLFPANFC GSVTQ
SEQ ID NO: 10: UspA2 from ATCC 25238
MKTMKLLPLKIAVTSAMIIGLGAASTANAQAKNDITLEDLPYLIKKIDQNELEADIGDIT
ALEKYLALSQYGNILALEELNKALEELDEDVGWNQNDIANLEDDVETLTKNQNALAEQGE
AIKEDLQGLADFVEGQEGKILQNETSIKKNTQRNLVNGFEIEKNKDAIAKNNESIEDLYD
FGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQA
NIQDLATYNELQDQYAQKQTEAIDALNKASSENTQNIEDLAAYNELQDAYAKQQTEAIDA
LNKASSENTQNIEDLAAYNELQDAYAKQQTEAIDALNKASSENTQNIAKNQADIANNINN
IYELAQQQDQHSSDIKTLAKASAANTDRIAKNKADADASFETLTKNQNTLIEKDKEHDKL
ITANKTAIDANKASADTKFAATADAITKNGNAITKNAKSITDLGTKVDGFDSRVTALDTK
VNAFDGRITALDSKVENGMAAQAALSGLFQPYSVGKFNATAALGGYGSKSAVAIGAGYRV
NPNLAFKAGAAINTSGNKKGSYNIGVNYEF
SEQ ID NO: 11: MC-001 (protein)-(M)(UspA2 amino acids 30-540)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKASHHHHHH
SEQ ID NO: 12 MC-002 (Protein)-(M)(UspA2 amino acids 30-540)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQD
AYAKQQTEAIDALNKASSENTQNIEDLAAYNELQDAYAKQQTEAIDALNKASSENTQNIAKNQADIANN
INNIYELAQQQDQHSSDIKTLAKASAANTDRIAKNKADADASFETLTKNQNTLIEKDKEHDKLITANKTAI
DANKASADTKFAATADAITKNGNAITKNAKSITDLGTKVDGFDSRVTALDTK
SEQ ID NO: 13 MC-003 (Protein)-(M)(UspA2 amino acids 30-540)(H)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKH
SEQ ID NO: 14 MC-004 (Protein)-(M)(UspA2 amino acids 30-540)(HH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKHH
SEQ ID NO: 15 MC-005 (Protein)-(M)(UspA2 amino acids 30-519)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSASHHHHHH
SEQ ID NO: 16 MC-006 (Protein)-(M)(UspA2 amino acids 30-519)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KS
SEQ ID NO: 17 MC-007 (Protein)-(M)(UspA2 amino acids 30-564)(ASHHHHHH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAAASHHHHHH
SEQ ID NO: 18 MC-008 (Protein)-(M)(UspA2 30-564)(HH)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAAHH
SEQ ID NO: 19 MC-009 (Protein)-(M)(UspA2 31-564)(HH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADFVEGQEGKILQNETSIKKNTQRNLVNGFEIEKNKDAIA
KNNESIEDLYDFGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQANIQDLATYNE
LQDQYAQKQTEAIDALNKASSENTQNIEDLAAYNELQDAYAKQQTEAIDALNKASSENTQNIEDLAAY
NELQDAYAKQQTEAIDALNKASSENTQNIAKNQADIANNINNIYELAQQQDQHSSDIKTLAKASAANTD
RIAKNKADADASFETLTKNQNTLIEKDKEHDKLITANKTAIDANKASADTKFAATADAITKNGNAITKNAK
SITDLGTKVDGFDSRVTALDTKVNAFDGRITALDSKVENGMAAQAAHH
SEQ ID NO: 20 MC-010 (Protein)-(M)(UspA2 amino acids 30-564)
MQAKNDITLEDLPYLIKKIDQNELEADIGDITALEKYLALSQYGNILALEELNKALEELDEDVGWNQNDIA
NLEDDVETLTKNQNALAEQGEAIKEDLQGLADFVEGQEGKILQNETSIKKNTQRNLVNGFEIEKNKDAI
AKNNESIEDLYDFGHEVAESIGEIHAHNEAQNETLKGLITNSIENTNNITKNKADIQALENNVVEELFNLS
GRLIDQKADIDNNINNIYELAQQQDQHSSDIKTLKKNVEEGLLELSGHLIDQKTDIAQNQANIQDLATYN
ELQDQYAQKQTEAIDALNKASSENTQNIEDLAAYNELQDAYAKQQTEAIDALNKASSENTQNIEDLAA
YNELQDAYAKQQTEAIDALNKASSENTQNIAKNQADIANNINNIYELAQQQDQHSSDIKTLAKASAANT
DRIAKNKADADASFETLTKNQNTLIEKDKEHDKLITANKTAIDANKASADTKFAATADAITKNGNAITKNA
KSITDLGTKVDGFDSRVTALDTKVNAFDGRITALDSKVENGMAAQAA
SEQ ID NO: 21 MC-011 (Protein)-(M)(UspA2 amino acids 31-540)(ASHHHHHH)
MAKNDITLEDLPYLIKKIDQNELEADIGDITALEKYLALSQYGNILALEELNKALEELDEDVGWNQNDIAN
LEDDVETLTKNQNALAEQGEAIKEDLQGLADFVEGQEGKILQNETSIKKNTQRNLVNGFEIEKNKDAIA
KNNESIEDLYDFGHEVAESIGEIHAHNEAQNETLKGLITNSIENTNNITKNKADIQALENNVVEELFNLSG
RLIDQKADIDNNINNIYELAQQQDQHSSDIKTLKKNVEEGLLELSGHLIDQKTDIAQNQANIQDLATYNE
LQDQYAQKQTEAIDALNKASSENTQNIEDLAAYNELQDAYAKQQTEAIDALNKASSENTQNIEDLAAY
NELQDAYAKQQTEAIDALNKASSENTQNIAKNQADIANNINNIYELAQQQDQHSSDIKTLAKASAANTD
RIAKNKADADASFETLTKNQNTLIEKDKEHDKLITANKTAIDANKASADTKFAATADAITKNGNAITKNAK
SITDLGTKVDGFDSRVTALDTKASHHHHHH
SEQ ID NO: 22 UspA2 American 2933 (613 aa)
MKTMKLLPLKIAVTSAMIIGLGAASTANAQSRDRSLEDIQDSISKLVQDDINTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAENKKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 23 UspA2 American 2912 (644 aa)
MKTMKLLPLKIAVTSALIIGLGAASTANAQQQLQTETFLPNFLSNDNYDLTDPFYHNMILGDTALLDKQD
GSQPQLKFYSNDKDSVPDSLLFSKLLHEQQLNGFKKGDTIIPLDKDGKPVYQVDYKLDGKGKKQKRR
QVYSVTTKTATDDDVNSAYSRGILGKVDDLDDEMNFLNHDITSLYDVTANQQDAIKDLKKGVKGLNKE
LKELDKEVGVLSRDIGSLNDDVAQNNESIEDLYDFSQEVADSIGEIHAHNKAQNETLQDLITNSVENTN
NITKNKADIQALENNVVEELFNLSGRLIDQKADLTKDIKTLESNVEEGLLELSGHLIDQKADIAKNQADIA
QNQANIQDLAAYNELQDAYAKQQTEAIDALNKASSENTQNIEDLAAYNELQDAYAKQQTEAIDALNKA
SSENTQNIAKNQADIANNINNIYELAQQQDQHSSDIKTLAKASAANTDRIAKNKADADASFETLTKNQN
TLIEKDKEHDKLITANKTAIDENKASADTKFAATADAITKNGNAITKNAKSITDLGTKVDGFDSRVTALDT
KVNAFDGRITALDSKVENGMAAQAALSGLFQPYSVGKFNATAALGGYGSKSAVAIGAGYRVNPNLAF
KAGAAINTSGNKKGSYNIGVNYEF
SEQ ID NO: 24 UspA2 American 2908 (591 aa)
MKTMKLLPLKIAVTSALIVGLGAASTANAQLVERFFPNIFLDKPLAKQHYHNVVVGDTSIVSDLQSNSD
QLKFYSDDEGLVPDSLLFNKMLHEQLLNGFKEGDTIIPLDENGKPVYKVDYKLDGKEPRKVYSVTTKIA
TAEDVATSSYANGIQKDIDDLYDFDHQVTERLTQHGKTIYRNGERILANEESVQYLNKEVQNNIEHIYE
LAQQQDQHSSDIKTLESNVEKGLLELSGHLIDQKADLTKDIKTLESNVEEGLLDLSGRLIDQKADLTKDI
KTLESNVEEGLLDLSGRLIDQKADIAQNQANIQDLAAYNELQDQYAQKQTEAIDALNKASSENTQNIED
LAAYNELQDAYAKQQTEAIDALNKASSENTQNIAKNQADIANNINNIYELAQQQDQHSSDIKTLAKASA
ANTNRIATAELGIAENKKDAQIAKAQANANKTAIDENKASADTKFAATADAITKNGNAITKNAKSITDLGT
KVDGFDSRVTALDTKVNAFDGRITALDSKVENGMAAQAALSGLFQPYSVGKFNATAALGGYGSKSAV
AIGAGYRVNPNLAFKAGAAINTSGNKKGSYNIGVNYEF
SEQ ID NO: 25 UspA2 Finnish 307 (687 aa)
MKTMKLLPLKIAVTSAMIIGLGAASTANAQQQQQQQQQQQSRTEIFFPNIFFNENHDELDDAYHNIILG
DTALLDKQDGSQPQLKFYSNDKDSVPDSLLFSKLLHEQQLNGFKKGDTIIPLDKDGKPVYQVDYKLDG
KGKKQKRRQVYSVTTKTATDDDVNSAYSRGILGKVDDLDDEMNFLNHDITSLYDVTANQQDAIKGLKK
GVKGLNKELKELDKEVGVLSRDIGSLNDDVAQNNESIEDLYDFSQEVADSIGEIHAHNKAQNETLQDLI
TNSVENTNNITKNKADIQALENNVVEELFNLSGRLIDQKADLTKDIKTLESNVEEGLLELSGHLIDQKADI
AKNQADIAQNQANIQDLAAYNELQDAYAKQQTEAIDALNKASSENTQNIEDLAAYNELQDAYAKQQTE
AIDALNKASSENTQNIEDLAAYNELQDAYAKQQTEAIDALNKASSENTQNIAKNQADIANNINNIYELAQ
QQDQHSSDIKTLAKASAANTDRIAKNKADADASFETLTKNQNTLIEKDKEHDKLITANKTAIDENKASAD
TKFAATADAITKNGNAITKNAKSITDLGTKVDAFDGRVTALDTKVNAFDGRITALDSKVENGMAAQAAL
SGLFQPYSVGKFNATAALGGYGSKSAVAIGAGYRVNPNLAFKAGAAINTSGNKKGSYNIGVNYEF
SEQ ID NO: 26 UspA2 Finnish 353 (683 amino acids)
MKTMKLLPLKIAVTSAMIVGLGMASTANAQQQKSPKTETFLPNIFFNEYADDLDTLYHNMILGDTAITH
DDQYKFYADDATEVPDSLFFNKILHDQLLYGFKEGDKIIPLDENGKPVYKLDKRLENGVQKTVYSVTTK
TATADDVNSAYSRGIQGDIDDLYEANKENVNRLIEHGDKIFANEESVQYLNREVQNNIENIHELAQQQD
QHSSDIKTLKKNVEKDLLDLSGRLIAQKEDIAQNQTDIQDLATYNELQDQYAQKQTEAIDALNKASSEN
TQNIAKNSNHIKTLENNIEEGLLELSGHLIDQKADLTKDIKALESNVEEGLLDLSGRLIDQKADIAQNQAN
IQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 27 UspA2 Finnish 358 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGGTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 28 UspA2 Finnish 216 (684 amino acids)
MKTMKLLPLKIAVTSAMIIGLGAASTANAQQQQKTKTEVFLPNLFDNDYYDLTDPLYHSMILGDTATLF
DQQDNSKSQLKFYSNDKDSVPDSLLFSKLLHEQQLNGFKAGDTIIPLDKDGKPVYTQDTRTKDGKVET
VYSVTTKIATQDDVEQSAYSRGIQGDIDDLYDINREVNEYLKATHDYNERQTEAIDALNKASSANTDRI
DTAEERIDKNEYDIKALESNVGKDLLDLSGRLIAQKEDIDNNINHIYELAQQQDQHSSDIKTLKNNVEEG
LLELSGHLIDQKADLTKDIKTLENNIEEGLLELSGHLIDQKADLTKDIKTLENNIEEGLLELSGHLIDQKAD
IAQNQANIQDLAAYNELQDQYAQKQTEAIDALNKASSENTQNIEDLAAYNELQDAYAKQQTEAIDALNK
ASSENTQNIEDLAAYNELQDAYAKQQTEAIDALNKASSENTQNIAKNQADIANNINNIYELAQQQDQHS
SDIKTLAKVSAANTDRIAKNKADADASFETLTKNQNTLIEKDKEHDKLITANKTAIDANKASADTKFAAT
ADAITKNGNAITKNAKSITDLGTKVDGFDGRVTALDTKVNAFDGRITALDSKVENGMAAQAALSGLFQP
YSVGKFNATAALGGYGSKSAVAIGAGYRVNPNLAFKAGAAINTSGNKKGSYNIGVNYEF
SEQ ID NO: 29 UspA2 Dutch H2 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKKGDTIIPLDENGKPVYKLDSIVEQGKTKTVYSVTT
KTATADDVNSAYSRGIQGDIDDLYEANKENVNRLIEHGDKIFANEESVQYLNREVQNNIENIYELVQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 30 UspA2 Dutch F10 (574 amino acids)
MKTMKLLPLKIAVTSAMIIGLGAASTANAQLAEQFFPNIFSNHAPVKQHYHNVVVGDTSIVENLQDSDD
TQLKFYSNDEYSVPDSLLFNKMLHEQQLNGFKKGDTIIPLDENGKPVYKVDYKLDGQEPRRVYSVTTK
IATQDDVDNSPYSRGIQGDIDDLYEANKENVNRLIEHGDKIFANEESVQYLNKEVQNNIENIYELAQQQ
DQHSSDIKTLKKNVEEGLLELSGHLIDQKADLTKDIKTLESNVEEGLLELSGHLIDQKADIAKNQADIAQ
NQANIQDLAAYNELQDAYAKQQTEAIDALNKASSENTQNIEDLAAYNELQDAYAKQQTEAIDALNKAS
SENTQNIAKNQADIANNINNIYELAQQQDQHSSDIKTLAKASAANTDRIAKNKADADASFETLTKNQNT
LIEKDKEHDKLITANKTAIDANKASADTKFAATADAITKNGNAITKNAKSITDLGTKVDAFDGRVTALDTK
VNAFDGRITALDSKVENGMAAQAALSGLFQPYSVGKFNATAALGGYGSKSAVAIGAGYRVNPNLAFK
AGAAINTSGNKKGSYNIGVNYEF
SEQ ID NO: 31 UspA2 Norwegian 1 (678 amino acids)
MKTMKLLPLKIAVTSALIVGLGAASTANAQQQPQTETFFPNIFFNENHDALDDVYHNMILGDTAITQDN
QYKFYADAISEVPDSLLFNKILHDQQLNGFKEGDTIIPLDENGKPVYKLDEKVENGVKKSVYSVTTKTA
TRADVEQSAYSRGIQGDIDDLYEANKENVNRLIEHGDKIFANEESVQYLNKEVQNNIENIHELAQQQD
QHSSDIKTLKKNVEEGLLELSGHLIDQKADLTKDIKTLESNVEEGLLDLSGRLLDQKADIAQNQANIQDL
AAYNELQDAYAKQQTEAIDALNKASSENTQNIEDLAAYNELQDAYAKQQTEAIDALNKASSENTQNIE
DLAAYNELQDAYAKQQTEAIDALNKASSENTQNIEDLAAYNELQDAYAKQQTEAIDALNKASSENTQNI
EDLAAYNELQDAYAKQQTEAIDALNKASSENTQNIAKNQADIANNINNIYELAQQQDQHSSDIKTLAKA
SAANTDRIAKNKADADASFETLTKNQNTLIEKDKEHDKLITANKTAIDANKASADTKFAATADAITKNGN
AITKNAKSITDLGTKVDAFDGRVTALDTKVNALDTKVNAFDGRITALDSKVENGMAAQAALSGLFQPYS
VGKFNATAALGGYGSKSAVAIGAGYRVNPNLAFKAGAAINTSGNKKGSYNIGVNYEF
SEQ ID NO: 32 UspA2 Norwegian 13 (678 amino acids)
MKTMKLLPLKIAVTSAMIVGLGAASTANAQQQQQPRTETFFPNIFFNENHDALDDVYHNMILGDTAITQ
DNQYKFYADAISEVPDSLLFNKILHDQQLNGFKEGDTIIPLDENGKPVYKLDEKVENGVKKSVYSVTTK
TATRADVEQSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKTLENNIEEGLLELSGHLIDQKADLTKDIKA
LESNVEEGLLDLSGRLLDQKADIAQNQANIQDLAAYNELQDQYAQKQTEAIDALNKASSENTQNIEDL
AAYNELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDTNKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 33 UspA2 Norwegian 33 (587 amino acids)
MKTMKLLPLKIAVTSALIVGLGAASTANAQLVERFFPNIFLDKPLAKQHYHNVVVGDTSIVSDLQSNSD
QLKFYSDDEGLVPDSLLFNKMLHEQLLNGFKEGDTIIPLDENGKPVYKVDYKLDGKEPRKVYSVTTKIA
TAEDVATSSYANGIQKDIDDLYDFDHQVTERLTQHGKTIYRNGERILANEESVQYLNKEVQNNIEHIYE
LAQQQDQHSSDIKTLESNVEKGLLELSGHLIDQKADLTKDIKTLENNVEEGLLDLSGRLIDQKADIAQN
QANIQDLAAYNELQDQYAQKQTEAIDALNKASSENTQNIEDLAAYNELQDAYAKQQTEAIDALNKASS
ENTQNIAKNQADIANNINNIYELAQQQDQHSSDIKTLAKASAANTDRIAKNKADADASFETLTKNQNTLI
EKDKEHDKLITANKTAIDTNKASADTKFAATADAITKNGNAITKNAKSITDLGTKVDGFDSRVTALDTKV
NALDTKVNALDTKVNAFDGRITALDSKVENGMAAQAALSGLFQPYSVGKFNATAALGGYGSKSAVAI
GAGYRVNPNLAFKAGAAINTSGNKKGSYNIGVNYEF
SEQ ID NO: 34 UspA2 Norwegian 25 (678 amino acids)
MKTMKLLPLKIAVTSAMIVGLGAASTANAQQQQQPRTETFFPNIFFNENHDALDDVYHNMILGDTAITQ
DNQYKFYADAISEVPDSLLFNKILHDQQLNGFKEGDTIIPLDENGKPVYKLDEKVENGVKKSVYSVTTK
TATRADVEQSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKTLENNIEEGLLELSGHLIDQKADLTKDIKA
LESNVEEGLLDLSGRLLDQKADIAQNQANIQDLAAYNELQDQYAQKQTEAIDALNKASSENTQNIEDL
AAYNELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDTNKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 35 UspA2 Norwegian 27 (616 amino acids)
MKTMKLLPLKIAVTSALIVGLGAASTANAQVRDKSLEDIEALLGKIDISKLEKEKKQQTELQKYLLLSQYA
NVLTMEELNKNVEKNTNSIEALGYEIGWLENDIADLEEGVEELTKNQNTLIEKDEEHDRLIAQNQADIKT
LENNVVEELFNLSDRLIDQEADIAKNNASIEELYDFDNEVAERIGEIHAYTEEVNKTLEKLITNSVKNTDN
IDKNKADIQALENNVEEGLLELSGHLIDQKADLTKDIKALESNVEEGLLDLSGRLLDQKADIAKNQADIA
QNQTDIQDLAAYNELQDQYAQKQTEAIDALNKASSENTQNIEDLAAYNELQDAYAKQQTEAIDALNKA
SSENTQNIEDLAAYNELQDAYAKQQTEAIDALNKASSENTQNIAKNQADIANNINNIYELAQQQDQHSS
DIKTLAKVSAANTDRIAKNKADADASFETLTKNQNTLIEKDKEHDKLITANKTAIDANKASADTKFAATA
DAITKNGNAITKNAKSITDLGTKVDGFDSRVTALDTKVNAFDGRITALDSKVENGMAAQAALSGLFQPY
SVGKFNATAALGGYGSKSAVAIGAGYRVNPNLAFKAGAAINTSGNKKGSYNIGVNYEF
SEQ ID NO: 36 UspA2 Norwegian 36 (676 amino acids)
MKTMKLLPLKIAVTSALIVGLGAASTANAQATETFLPNLFDNDYTETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGHLIDQKADLTKDIKALESNVEEGLLDLSGHLIDQKADLTKDIKALESNVEEGL
LDLSGRLLDQKADIAKNQADIAQNQTDIQDLAAYNELQDQYAQKQTEAIDALNKASSENTQNIEDLAAY
NELQDQYAQKQTEAIDALNKASSENTQNIEDLAAYNELQDQYAQKQTEAIDALNKASSENTQNIEDLA
AYNELQDQYAQKQTEAIDALNKASSENTQNIAKNQADIANNINNIYELAQQQDQHSSDIKTLAKASAAN
TDRIAKNKADADASFETLTKNQNTLIEKDKEHDKLITANKTAIDANKASADTKFAATADAITKNGNAITKN
AKSITDLGTKVDGFDGRVTALDTKVNALDTKVNAFDGRITALDSKVENGMAAQAALSGLFQPYSVGKF
NATAALGGYGSKSAVAIGAGYRVNPNLAFKAGAAINTSGNKKGSYNIGVNYEF
SEQ ID NO: 37 UspA2 BC5SV (629 amino acids)
MKTMKLLPLKIAVTSALIVGLGAASTANAQNGTSTKLKNLKEYAQYLDNYAQYLDDDIDDLDKEVGELS
QNIAKNQANIKDLNKKLSRDIDSLREDVYDNQYEIVNNQADIEKNQDDIKELENNVGKELLNLSGRLLD
QKADIDNNINNIYELAQQQDQHSSDIKTLKKNVEEGLLELSGHLIDQKSDIAQNQTDIQDLATYNELQD
QYAQKQTEAIDALNKASSENTQNIEDLAAYNELQDAYAKQQTEAIDALNKASSENTQNIQDLAAYNEL
QDAYAKQQTEAIDALNKASSENTQNIEDLAAYNELQDAYAKQQTEAIDALNKASSENTQNIEDLAAYN
ELQDAYAKQQTEAIDALNKASSENTQNIEDLAAYNELQDAYAKQQTEAIDALNKASSENTQNIAKNQA
DIANNINNIYELAQQQDQHSSDIKTLAKASAANTDRIAKNKADADASFETLTKNQNTLIEKDKEHDKLITA
NKTAIDANKASADTKFAATADAITKNGNAITKNAKSITDLGTKVDAFDGRVTALDTKVNAFDGRITALDS
KVENGMAAQAALSGLFQPYSVGKFNATAALGGYGSKSAVAIGAGYRVNPNLAFKAGAAINTSGNKKG
SYNIGVNYEF
SEQ ID NO: 38 UspA2 Norwegian 14 (683 amino acids)
MKTMKLLPLKIAVTSAMIVGLGMASTANAQQQRSPKTETFLPNIFFNEYADDLDTLYHNMILGDTAITH
DDQYKFYADDATEVPDSLFFNKILHDQLLYGFKEGDKIIPLDENGKPVYKLDKRLDNGVQKTVYSVTTK
TATADDVNSAYSRGIQGDIDDLYEANKENVNRLIEHGDKIFANEESVQYLNKEVQNNIENIHELAQQQD
QHSSDIKTLKKNVEEGLLELSGHLIDQKTDIAQNQTDIQDLATYNELQDQYAQKQTEAIDALNKASSEN
TQNIAKNSNRIKALENNIEEGLLELSGHLIDQKADLTKDIKALESNVEEGLLDLSGRLIDQKADIAQNQAN
IQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 39 UspA2 Norwegian 3 (700 amino acids)
MKTMKLLPLKIAVTSAMIVGLGAASTANAQAQSNRSLDQVQALLRGIDETKIKKEIQQSQQPELNKYLT
FNQLANALNIEELNNNVQKNTQRLDSAATLYGDLSKTVPKSIKENKESIKENKESIKENKESIKENKESI
KENKESIKENKESITTLTRKSFQNQVDIVRNNASIEDLYAYGQEVAKSIGEIHAYTEEVNKTLENLITNSV
ENTNNITKNKADIQALENNVVEELFNLSGRLIDQKADIDNNINNIYELAQQQDQHSSDIKTLKKNVEEGL
LELSGHLIDQKADLTKDIKTLESNVEEGLLDLSGRLLDQKADIAQNQANIQDLAAYNELQDAYAKQQTE
AIDALNKASSENTQNIEDLAAYNELQDAYAKQQTEAIDALNKASSENTQNIEDLAAYNELQDAYAKQQT
EAIDALNKASSENTQNIEDLAAYNELQDAYAKQQTEAIDALNKASSENTQNIAKNQADIANNINNIYELA
QQQDQHSSDIKTLAKASAANTDRIAKNKADADASFETLTKNQNTLIEKDKEHDKLITANKTVIDANKAS
ADTKFAATADAITKNGNAITKNAKSITDLGTKVDGFDGRVTALDTKVNALDTKVNAFDGRITALDSKVE
NGMAAQAALSGLFQPYSVGKFNATAALGGYGSKSAVAIGAGYRVNPNLAFKAGAAINTSGNKKGSYN
IGVNYEF
SEQ ID NO: 40 UspA2 Finnish 414 (676 amino acids)
MKTMKLLPLKIAVTSALIVGLGAASTANAQATETFLPNLFDNDYIETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGHLIDQKADLTKDIKTLENNVEEGLLELSGHLIDQKADLTKDIKALESNVEEGL
LDLSGRLLDQKADIAKNQADIAQNQTDIQDLAAYNELQDQYAQKQTEAIDALNKASSENTQNIEDLAAY
NELQDQYAQKQTEAIDALNKASSENTQNIEDLAAYNELQDQYAQKQTEAIDALNKASSENTQNIEDLA
AYNELQDQYAQKQTEAIDALNKASSENTQNIAKNQADIANNINNIYELAQQQDQHSSDIKTLAKASAAN
TDRIAKNKADADASFETLTKNQNTLIEKDKEHDKLITANKTAIDANKASADTKFAATADAITKNGNAITKN
AKSITDLGTKVDGFDGRVTALDTKVNALDTKVNAFDGRITALDSKVENGMAAQAALSGLFQPYSVGKF
NATAALGGYGSKSAVAIGAGYRVNPNLAFKAGAAINTSGNKKGSYNIGVNYEF
SEQ ID NO: 41 UspA2 Japanese Z7476 (678 amino acids)
MKTMKLLPLKIAVTSAMIIGLGAASTANAQLAEQFFPNIFSNHAPVKQHYHNVVVGDTSIVENLQDSDD
TQLKFYSNDEYSVPDSLLFNKMLHEQQLNGFKKGDTIIPLDENGKPVYKVDYKLDGQEPRRVYSVTTK
IATQDDVDNSPYSRGIQGDIDDLYEANKENVNRLIEHGDKIFANEESVQYLNKEVQNNIENIYELAQQQ
DQHSSDIKTLKKNVEEGLLELSGRLIDQKADIAQNQANIQDLAAYNELQDQYAQKQTEAIDALNKASSE
NTQNIEDLAAYNELQDAYAKQQTEAIDALNKASSENTQNIEDLAAYNELQDAYAKQQTEAIDALNKASS
ENTQNIEDLAAYNELQDAYAKQQTEAIDALNKASSENTQNIEDLAAYNELQDAYAKQQTEAIDALNKAS
SENTQNIEDLAAYNELQDAYAKQQTEAIDALNKASSENTQNIAKNQADIANNINNIYELAQQQDQHSSD
IKTLAKVSAANTDRIAKNKADADASFETLTKNQNTLIEKDKEHDKLITANKTAIDANKASADTKFAATAD
AITKNGNAITKNAKSITDLGTKVDGFDGRVTALDTKVNAFDGRITALDSKVENGMAAQAALSGLFQPYS
VGKFNATAALGGYGSKSAVAIGAGYRVNPNLAFKAGAAINTSGNKKGSYNIGVNYEF
SEQ ID NO: 42 UspA2 Belgian Z7530 (613 amino acids)
MKTMKLLPLKIAVTSAMIIGLGAASTANAQSRDRSLEDIQDSISKLVQDDINTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAENKKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 43 German Z8063 (589 amino acids)
MKTMKLLPLKIAVTSALIVGLGAASTANAQATNKDITLEDVLKSIEEIDPYELRDYIEYPTAIERFLLLSQY
GNTLTLEEFDNDIELLDQDVEDLEESVTELAKNQNSLIEQGEAIKEDLQGLADFVERQEDKILQNETSIK
KNTQRNLVNGFEIEKNKDAIAKNNESIEDLYDFGHEVAKSIGEIHAHNEAQNETLKDLITNSVKNTDNIT
KNKADIQALESNVEKGLLELSGHLIDQKADIDNNINNIHELAQQQDQHSSDIKTLKKNVEEGLLELSGHL
IDQKSDIAQNQANIQDLATYNELQDQYAQKQTEAIDALNKASSENTQNIEDLAAYNELQDAYAKQQTE
AIDALNKASSENTQNIAKNQADIANNINNIYELAQQQDQHSSDIKTLAKASAANTDRIAKNKADADASFE
TLTKNQNTLIEKDKEHDKLITANKTAIDANKASADTKFAATADAITKNGNAITKNAKSITDLGTKVDGFDS
RVTALDTKVNAFDGRITALDSKVENGMAAQAALSGLFQPYSVGKFNATAALGGYGSKSAVAIGAGYR
VNPNLAFKAGAAINTSGNKKGSYNIGVNYEF
SEQ ID NO: 44 UspA2 American O12E (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGDTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 45 UspA2 Greek MC317 (650 amino acids)
MKTMKLLPLKIAVTSALIVGLGAASTANAQQQQKTKTEVFLPNLFYNDYIEETDLLYHNMILGDTAALVD
RQNYSNSQLKFYSNDEESVPDSLLFSKMLNNQQLNGFKAGDIIIPVDANGQVIYQKDTRVEGGKTRTV
LSVTTKIATQQDVDSAYSRGIQGKVNDLDDEMNFLNHDITSLYDVTANQQDDIKGLKKGVKDLKKGVK
GLNKELKELDKEVGVLSRDIGSLNDDVAQNNESIEDLYDFSQEVADSIGEIHAHNKAQNETLQDLITNS
VENTNNITKNKADIQALENNVVEELFNLSGRLIDQKADLTKDIKTLESNVEEGLLELSGHLIDQKADIAKN
QADIAQNQANIQDLAAYNELQDAYAKQQTEAIDALNKASSENTQNIEDLAAYNELQDAYAKQQTEAID
ALNKASSENTQNIAKNQADIANNINNIYELAQQQDQHSSDIKTLAKASAANTDRIAKNKADADASFETLT
KNQNTLIEKDKEHDKLITANKTAIDENKASADTKFAATADAITKNGNAITKNAKSITDLGTKVDGFDGRV
TALDTKVNAFDGRITALDSKVENGMAAQAALSGLFQPYSVGKFNATAALGGYGSKSAVAIGAGYRVN
PNLAFKAGAAINTSGNKKGSYNIGVNYEF
SEQ ID NO: 46 UspA2 American V1122 (616 amino acids)
MKTMKLLPLKIAVTSALIVGLGAVSTTNAQAQSRSLDQIQTKLADLAGKIAAGKNGGGQNNQNNQNDI
NKYLFLSQYANILTMEELNNNVVKNSSSIETLETDFGWLENDVADLEDGVEELTKNQNTLIEKDEEHDR
LIAQNQADIQTLENNVVEELFNLSDRLIDQKADIAKNQADIAQNNESIEELYDFDNEVAEKIGEIHAYTEE
VNKTLQDLITNSVKNTDNIDKNKADIDNNINHIYELAQQQDQHSSDIKTLKNNVEEGLLELSGHLIDQKA
DLTKDIKTLENNVEEGLLDLSGRLIDQKADIAKNQADIAQNQTDIQDLAAYNELQDQYAQKQTEAIDALN
KASSENTQNIEDLAAYNELQDAYAKQQTEAIDALNKASSENTQNIAKNQADIANNINNIYELAQQQDQH
SSDIKTLAKASAANTDRIAKNKADADASFETLTKNQNTLIEKDKEHDKLITANKTAIDENKASADTKFAA
TADAITKNGNAITKNAKSITDLGTKVDGFDGRVTALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 47 UspA2 American P44 (668 amino acids)
MKTMKLLPLKIAVTSALIVGLGTASTANAQVASPANQKIQQKIKKVRKELRQDIKSLRNDIDSNTADIGS
LNDDVADNQDDILDNQADIAKNQDDIEKNQADIKELDKEVGVLSREIGSLNDDIADNYTDIIDNYTDIIDN
QANIAKNQDDIEKNQADIKELDKEVGVLSREIGSLNDDVADNQDDIAKNQADIQTLENNVEEGLLELSG
HLLDQKADIDNNINNIYELAQQQDQHSSDIKTLKKNVEEGLLELSGHLIDQKTDIAQNQANIQDLATYNE
LQDQYAQEQTEAIDALNKASSENTQNIAKNSNRIKALESNVEEGLLELSGHLIDQKADLTKDIKALESNV
EEGLLELSGHLIDQKADIAQNQANIQDLAAYNELQDQYAQKQTEAIDALNKASSENTQNIEDLAAYNEL
QDAYAKQQTEAIDALNKASSENTQNIAKNQADIANNINNIYELAQQQDQHSSDIKTLAKASAANTDRIA
KNKADADASFETLTKNQNTLIEKDKEHDKLITANKTAIDANKVSADTKFAATADAITKNGNAITKNAKSIT
DLGTKVDAFDSRVTALDTKVNAFDGRITALDSKVENGMAAQAALSGLFQPYSVGKFNATAALGGYGS
KSAVAIGAGYRVNPNLAFKAGAAINTSGNKKGSYNIGVNYEF
SEQ ID NO: 48 UspA2 American V1171 (674 amino acids)
MKTMKLLPLKIAVTSAMIVGLGATSTVNAQVVEQFFPNIFFNENHDELDDAYHNMILGDTAIVSNSQDN
STQLKFYSNDEDSVPDSLLFSKLLHEQQLNGFKAGDTIIPLDKDGKPVYTKDTRTKDGKVETVYSVTTK
IATQDDVEQSAYSRGIQGDIDDLYDINREVNEYLKATHDYNERQTEAIDALNKASSANTDRIDTAEERID
KNEYDIKALESNVEEGLLELSGHLIDQKADLTKDIKALESNVEEGLLELSGHLIDQKADLTKDIKALESNV
EEGLLDLSGRLIDQKADIAQNQANIQDLAAYNELQDAYAKQQTEAIDALNKASSENTQNIEDLAAYNEL
QDAYAKQQTEAIDALNKASSENTQNIEDLAAYNELQDAYAKQQTEAIDALNKASSENTQNIEDLAAYN
ELQDAYAKQQTEAIDALNKASSENTQNIAKNQADIANNINNIYELAQQQDQHSSDIKTLAKASAANTDRI
AKNKADADASFETLTKNQNTLIEKDKEHDKLITANKTAIDANKASADTKFAATADAITKNGNAITKNAKSI
TDLGTKVDGFDGRVTALDTKVNALDTKVNAFDGRITALDSKVENGMAAQAALSGLFQPYSVGKFNAT
AALGGYGSKSAVAIGAGYRVNPNLAFKAGAAINTSGNKKGSYNIGVNYEF
SEQ ID NO: 49 UspA2 American TTA24 (613 amino acids)
MKTMKLLPLKIAVTSAMIIGLGAASTANAQSRDRSLEDIQDSISKLVQDDIDTLKQDQQKMNKYLLLNQL
ANTLITDELNNNVIKNTNSIEALGDEIGWLENDIADLEEGVEELTKNQNTLIEKDEEHDRLIAQNQADIQT
LENNVVEELFNLSGRLIDQEADIAKNNASIEELYDFDNEVAERIGEIHAYTEEVNKTLENLITNSVKNTDN
IDKNKADIDNNINHIYELAQQQDQHSSDIKTLKNNVEEGLLELSGHLIDQKADLTKDIKALESNVEEGLL
DLSGRLLDQKADLTKDIKALESNVEEGLLDLSGRLLDQKADIAQNQTDIQDLAAYNELQDQYAQKQTE
AIDALNKASSENTQNIEDLAAYNELQDAYAKQQTEAIDALNKASSENTQNIAKNQADIANNINNIYELAQ
QQDQHSSDIKTLAKASAANTNRIATAELGIAENKKDAQIAKAQANANKTAIDENKASADTKFAATADAIT
KNGNAITKNAKSITDLGTKVDGFDGRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
SEQ ID NO: 50 UspA2 American O35E (576 amino acids)
MKTMKLLPLKIAVTSAMIVGLGATSTVNAQVVEQFFPNIFFNENHDELDDAYHNMILGDTAIVSNSQDN
STQLKFYSNDEDSVPDSLLFSKLLHEQQLNGFKAGDTIIPLDKDGKPVYTKDTRTKDGKVETVYSVTTK
IATQDDVEQSAYSRGIQGDIDDLYDINREVNEYLKATHDYNERQTEAIDALNKASSANTDRIDTAEERID
KNEYDIKALESNVEEGLLELSGHLIDQKADLTKDIKALESNVEEGLLELSGHLIDQKADLTKDIKALESNV
EEGLLDLSGRLLDQKADIAKNQADIAQNQTDIQDLAAYNELQDAYAKQQTEAIDALNKASSENTQNIAK
NQADIANNINNIYELAQQQDQHSSDIKTLAKASAANTDRIAKNKADADASFETLTKNQNTLIEKDKEHD
KLITANKTAIDANKASADTKFAATADAITKNGNAITKNAKSITDLGTKVDGFDGRVTALDTKVNALDTKV
NAFDGRITALDSKVENGMAAQAALSGLFQPYSVGKFNATAALGGYGSKSAVAIGAGYRVNPNLAFKA
GAAINTSGNKKGSYNIGVNYEF
SEQ ID NO: 51 UspA2 American SP12-6 (684 amino acids)
MKTMKLLPLKIAVTSAMMVGLGMASTANAQQQKSPKTEIFLPNLFDNDNTELTDPLYHNMILGNTALLT
QENQYKFYADDGNGVPDSLLFNKILHDQLLHGFKEGDTIIPLDENGKPVYKLDSIVEQGKTKTVYSVTT
KTATADDVNSAYSRGIQGDIDDLYEANKENVNRLIEHGDKIFANEESVQYLNREVQNNIENIHELAQQQ
DQHSSDIKTLKKNVEKDLLDLSGRLIAQKEDIAQNQTDIQDLATYNELQDQYAQKQTEAIDALNKASSE
NTQNIAKNSNHIKTLENNIEEGLLELSGHLIDQKADLTKDIKALESNVEEGLLDLSGRLIDQKADIAQNQA
NIQDLAAYNELQDAYAKQQTEAIDALNKASSENTQNIEDLAAYNELQDAYAKQQTEAIDALNKASSENT
QNIEDLAAYNELQDAYAKQQTEAIDALNKASSENTQNIAKNQADIANNINNIYELAQQQDQHSSDIKTLA
KASAANTDRIAKNKADADASFETLTKNQNTLIEKDKEHDKLITANKTAIDANKASADTKFAATADAITKN
GNAITKNAKSITDLGTKVDGFDGRVTALDTKVNALDTKVNAFDGRITALDSKVENGMAAQAALSGLFQ
PYSVGKFNATAALGGYGSKSAVAIGAGYRVNPNLAFKAGAAINTSGNKKGSYNIGVNYEF
SEQ ID NO: 52 UspA2 American SP12-5 (686 amino acids)
MKTMKLLPLKIAVTSAMIIGLGAASTANAQATETFLPNLFDNDYTETTDPLYHGMILGNTAITQDTQYKF
YAENGNEVPDSLFFNKILHDQQLNGFKEGDTIIPLDENGKPVYKLDEITENGVKRKVYSVTTKTATRED
VEQSAYSRGIQGDIDDLYEANKENVNRLIEHGDKIFANEESVQYLNKEVQNNIENIHELAQQQDQHSS
DIKTLKKNVEEGLLELSGRLIAQKEDIAQNQTDIQDLATYNELQDQYAQKQTEAIDALNKASSENTQNIA
KNSNHIKTLENNIEEGLLELSGHLIDQKADLTKDIKALESNVEEGLLDLSGRLLDQKADIAKNQADIAQN
QTDIQDLAAYNELQDQYAQKQTEAIDALNKASSENTQNIEDLAAYNELQDAYAKQQTEAIDALNKASS
ENTQNIEDLAAYNELQDAYAKQQTEAIDALNKASSENTQNIAKNQADIANNINNIYELAQQQDQHSSDI
KTLAKASAANTDRIAKNKADADASFETLTKNQNTLIEKDKEHDKLITANKTAIDANKASADTKFAATADA
ITKNGNAITKNAKSITDLGTKVDGFDGRVTALDTKVNALDTKVNAFDGRITALDSKVENGMAAQAALSG
LFQPYSVGKFNATAALGGYGSKSAVAIGAGYRVNPNLAFKAGAAINTSGNKKGSYNIGVNYEF
SEQ ID NO: 53 UspA2 Swedish BC5 (630 amino acids)
MKTMKLLPLKIAVTSAMIIGLGAASTANAQAKNDITLEDLPYLIKKIDQNELEADIGDITALEKYLALSQYG
NILALEELNKALEELDEDVGWNQNDIANLEDDVETLTKNQNALAEQGEAIKEDLQGLADFVEGQEGKIL
QNETSIKKNTQRNLVNGFEIEKNKDAIAKNNESIEDLYDFGHEVAESIGEIHAHNEAQNETLKGLITNSIE
NTNNITKNKADIQALENNVVEELFNLSGRLIDQKADIDNNINNIYELAQQQDQHSSDIKTLKKNVEEGLL
ELSGHLIDQKTDIAQNQANIQDLATYNELQDQYAQKQTEAIDALNKASSENTQNIEDLAAYNELQDAYA
KQQTEAIDALNKASSENTQNIEDLAAYNELQDAYAKQQTEAIDALNKASSENTQNIAKNQADIANNINNI
YELAQQQDQHSSDIKTLAKASAANTDRIAKNKADADASFETLTKNQNTLIEKDKEHDKLITANKTAIDAN
KASADTKFAATADAITKNGNAITKNAKSITDLGTKVDGFDSRVTALDTKVNAFDGRITALDSKVENGMA
AQAALSGLFQPYSVGKFNATAALGGYGSKSAVAIGAGYRVNPNLAFKAGAAINTSGNKKGSYNIGVN
YEF
SEQ ID NO: 54 UspA2 American 7169 (616 amino acids)
MKTMKLLPLKIAVTSALIVGLGAASTANAQAQDRSLEQIQDKLANLVEKIEQAKSQNGQSQKDINQYLL
LSQYANVLTMEELNNNVVKNSSSIETLDNDIAWLNDDLIDLDKEVGVLSRDIGSLHDDVAQNQADIKTL
KNNVVEELFNLSDRLIDQEADIAQNNESIEDLYDFGREVAESIGEIHAHNEAQNETLKDLITNSVKNTDN
ITKNKADIQALENDVGKELLNLSGRLIDQKADIDNNINHIYELAQQQDQHSSDIKTLKNNVEEGLLELSG
HLIDQKADLTKDIKALESNVEEGLLDLSGRLLDQKADIAQNQANIQDLAAYNELQDAYAKQQTEAIDAL
NKASSENTQNIEDLAAYNELQDAYAKQQTEAIDALNKASSENTQNIAKNQADIANNINNIYELAQQQDQ
HSSDIKTLAKASAANTDRIAKNKADADASFETLTKNQNTLIEKDKEHDKLITANKTAIDANKASADTKFA
ATADAITKNGNAITKNAKSITDLGTKVDGFDSRVTALDTKVNAFDGRITALDSKVENGMAAQAALSGLF
QPYSVGKFNATAALGGYGSKSAVAIGAGYRVNPNLAFKAGAAINTSGNKKGSYNIGVNYEF
SEQ ID NO: 55 UspA2 Finnish FIN2344 (614 amino acids)
MKTMKLLPLKIAVTSAMIIGLGATSTVNAQVVEQFFPNIFFNENHDELDDAYHNMILGDTAIVSNSQDNS
TQLKFYSNDEDSVPDSLLFSKLLHEQQLNGFKAGDTIIPLDKDGKPVYTKDTRTKDGKVETVYSVTTKI
ATQDDVEQSAYSRGIQGDIDDLYDINREVNEYLKATHDYNERQTEAIDALNKASSANTDRIDTAEERID
KNEYDIKALESNVGKDLLDLSGRLIAQKEDIDNNINHIYELAQQQDQHSSDIKTLKNNVEEGLLELSGHL
IDQKADLTKDIKTLESNVEEGLLDLSGRLIDQKADIAQNQANIQDLAAYNELQDQYAQKQTEAIDALNKA
SSENTQNIEDLAAYNELQDAYAKQQTEAIDALNKASSENTQNIAKNQADIANNINNIYELAQQQDQHSS
DIKTLAKVSAANTDRIAKNKADADASFETLTKNQNTLIEKDKEHDKLITANKTAIDANKASADTKFAATA
DAITKNGNAITKNAKSITDLGTKVDGFDGRVTALDTKVNAFDGRITALDSKVENGMAAQAALSGLFQPY
SVGKFNATAALGGYGSKSAVAIGAGYRVNPNLAFKAGAAINTSGNKKGSYNIGVNYEF
SEQ ID NO: 56 UspA2 American V1118 (679 amino acids)
MKTMKLPPLKIAVTSAMIIGLGAASTANAQTTETFLPNLFDNDYTETTDPLYHGMILGDTAITQDTQYKF
YAENGNEVPDSLFFNKILHDQLLNGFKAGDTIIPLDENGKPVYKLDERTENGVKRKVYSVTTKTATQAD
VEQSAYSRGIQGDIDDLYEANKENVNRLIEHGDKIFANEESVQYLNREVQNNIENIHELAQQQDQHSS
DIKTLKKNVEKDLLDLSGRLIAQKEDIAQNQTDIQDLATYNELQDQYAQKQTEAIDALNKASSENTQNIA
KNSNHIKTLENNIEECLLELSGHLIDQKADLTKDIKALESNVEEGLLDLSGRLIDQKADIAQNQANIQDLA
AYNELQDAYAKQQTEAIDALNKASSENTQNIEDLAAYNELQDAYAKQQTEAIDALNKASSENTQNIEDL
AAYNELQDAYAKQQTEAIDALNKASSENTQNIAKNQADIANNINNIYELAQQQDQHSSDIKTLAKASAA
NTDRIAKNKADADASFETLTKNQNTLIEKDKEHDKLITANKTAIDANKASADTKFAATADAITKNGNAITK
NAKSITDLGTKVDGFDGRVTALDTKVNALDTKVNAFDGRITALDSKVENGMAAQAALSGLFQPYSVGK
FNATAALGGYGSKSAVAIGAGYRVNPNLAFKAGAAINTSGNKKGSYNIGVNYEF
SEQ ID NO: 57 UspA2 American V1145 (724 amino acids)
MKTMKLLPLKIAVTSALIVGLGAASTANAQETLEEVLESIKQINEQDLQDDIGYNSALDRYLVLSQYGNL
LIAKELNENVEKNSNSIAKNSNSIADLEADVGYLAENQNTLIEQNETINQELEGITHELESFIAYAHAQDQ
KNLVNEFEIEKNKDAIAKNNESIEDLYDFGHEVAESIGEIHAYTEEVNKTLENLITNSVKNTDNITKNKADI
QALESNVEKELLNLSGRLIDQKADIDNNINHIYELAQQQDQHSSDIKTLKKNVEEGLLELSGHLIDQKSD
IAQNQTDIQDLATYNELQDQYAQKQTEAIDALNKASSENTQNIEDLAAYNELQDAYAKQQTEAIDALNK
ASSENTQNIEDLAAYNELQDAYAKQQTEAIDALNKASSENTQNIEDLAAYNELQDAYAKQQTEAIDALN
KASSENTQNIEDLAAYNELQDAYAKQQTEAIDALNKASSENTQNIEDLAAYNELQDAYAKQQTEAIDAL
NKASSENTQNIAKNQADIANNINNIYELAQQQDQHSSDIKTLAKASAANTDRIAKNKADADASFETLTK
NQNTLIEKDKEHDKLITANKTAIDANKASADTKFAATADAITKNGNAITKNAKSITDLGTKVDGFDSRVT
ALDTKVNAFDGRITALDSKVENGMAAQAALSGLFQPYSVGKFNATAALGGYGSKSAVAIGAGYRVNP
NLAFKAGAAINTSGNKKGSYNIGVNYEF
SEQ ID NO: 58 UspA2 American V1156 (611 amino acids)
MKTMKLLPLKIAVTSALIVGLGAASTANAQAQARDRSLEDIQALIGNIDVDKIRSQKQKNPEIFQYLLLN
QLSNTLITDELNNNVIKNTNSIETLDNDIAWLNDDLIDLDKEVGVLSRDIGSLHDDVAQNQADIKTLENN
VVEELFNLSDRLIDQEAEIAQNNESIEDLYDFGREVAESIGEIHAHNEAQNETLKDLITNSVKNTDNIDK
NKADIQALENNVEEGLLELSGHLIDQKADLTKDIKALESNVEEGLLDLSGRLLDQKADIAKNQADIAQN
QTDIQDLAAYNELQDQYAQKQTEAIDALNKASSENTQNIEDLAAYNELQDAYAKQQTEAIDALNKASS
ENTQNIEDLAAYNELQDAYAKQQTEAIDALNKASSENTQNIAKNQADIANNINNIYELAQQQDQHSSDI
KTLAKVSAANTDRIAKNKADADASFETLTKNQNTLIEKDKEHDKLITANKTAIDANKASADTKFAATADA
ITKNGNAITKNAKSITDLGTKVDGFDSRVTALDTKVNAFDGRITALDSKVENGMAAQAALSGLFQPYSV
GKFNATAALGGYGSKSAVAIGAGYRVNPNLAFKAGAAINTSGNKKGSYNIGVNYEF

1. An immunogenic composition comprising Protein E from Haemophilus influenzae or an immunogenic fragment thereof and PilA from Haemophilus influenzae or an immunogenic fragment thereof; a Protein D polypeptide; an UspA2 polypeptide; an anti-oxidant; and poloxamer.

2. The immunogenic composition according to claim 1, comprising 0.02 to 0.15%, 0.03 to 0.15%, 0.03 to 0.09%, 0.04 to 0.15%, 0.04 to 0.1%, 0.04 to 0.09%, 0.04 to 0.06% or 0.04 to 0.05% (w/v) poloxamer.

3. The immunogenic composition according to claim 1, comprising 0.1 to 20 mM, 0.1 to 15 mM, 0.5 to 15 mM, 5 to 15 mM, 7 to 12 mM, 8 to 12 mM or 8 to 10 mM antioxidant.

4. The immunogenic composition according to claim 1, comprising 15 to 30 μg/ml PE-PilA fusion protein, 15 to 30 μg/ml Protein D polypeptide and 6 to 9 μg/ml UspA2 polypeptide.

5. The immunogenic composition according to claim 1, comprising 9 to 15 μg PE-PilA fusion protein, 9 to 15 μg Protein D polypeptide, 3 to 5 μg UspA2 polypeptide.

6. The immunogenic composition according to claim 1, further comprising polysorbate 80, comprising 0.0001 to 0.03%, 0.0001 to 0.02%, 0.0001 to 0.01%, 0.0001 to 0.005%, 0.0001 to 0.002%, 0.0002 to 0.002%, 0.0003 to 0.0015% or 0.0004 to 0.0012% (w/v) polysorbate 80.

7. The immunogenic composition according to claim 1, in a solid form.

8. The immunogenic composition according to claim 7, suitable for reconstitution in an aqueous solution comprising an adjuvant, wherein said immunogenic composition after reconstitution is capable of generating an immune response against Haemophilus influenzae and/or Moraxella catarrhalis.

9. The immunogenic composition according to claim 1, in a liquid form, reconstituted with an aqueous solution comprising an adjuvant.

10. The immunogenic composition according to claim 1, further comprising sucrose.

11. The immunogenic composition according to claim 1, comprising 20 μg/ml PE-PilA fusion protein; 20 μg/ml Protein D polypeptide; 6.6 μg/ml UspA2 polypeptide; 0.5 to 1.5 mg/ml antioxidant; 0.2 to 0.6 mg/ml poloxamer; 20 to 60 mg/ml sucrose.

12. The immunogenic composition according to claim 11, further comprising polysorbate 80 in an amount less than 0.03% (w/v) polysorbate 80.

13. A process for preparing an immunogenic composition according to claim 1 comprising combining (i) Protein E from Haemophilus influenzae or an immunogenic fragment thereof and PilA from Haemophilus influenzae or an immunogenic fragment thereof; a Protein D polypeptide; and an UspA2 polypeptide; with (ii) an anti-oxidant and (iii) poloxamer.

14. A kit comprising (i) a first container comprising an immunogenic composition according to claim 1, in solid form and (ii) a second container comprising an adjuvant that is AS01E.

15. (canceled)

16. (canceled)

17. (canceled)

18. The immunogenic composition of claim 1, wherein Protein E and PilA are present as a PE-PilA fusion protein, wherein the PE-PilA fusion protein has 90% identity to SEQ ID NO: 9; the Protein D polypeptide has 90% identity to SEQ ID NO: 2; and the UspA2 polypeptide has 90% identity to SEQ ID NO: 19.

19. The immunogenic composition of claim 18, wherein the anti-oxidant is L-methionine and the poloxamer is poloxamer 188.

20. The immunogenic composition according to claim 9, wherein the adjuvant comprises AS01E.

21. The immunogenic composition according to claim 11, wherein the antioxidant is L-methionine in an amount of 1.2 mg/ml; the poloxamer is poloxamer 188 in an amount of 0.2 to 0.6 mg/ml; and the sucrose is in an amount of 40 mg/ml.

22. The immunogenic composition according to claim 21, comprising AS01E in a 0.5 ml dose.

23. The process of claim 13, wherein Protein E and PilA are present as a PE-PilA fusion protein, the antioxidant is L-methionine, and the poloxamer is poloxamer 188.

24. A method of treating or preventing a disease caused by H. influenzae and/or M. catarrhalis by administering the composition of claim 1 to a subject.


SEQUENCE LISTING (58 sequences)

SEQ ID NO: 1 - protein, 364 aa, H. influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 2 - protein, 348 aa, Artificial
  Protein D fragment with MDP tripeptide from NS1
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 3 - protein, 10 aa, H. influenzae
SSHSSNMANT

SEQ ID NO: 4 - protein, 160 aa, H. influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 5 - protein, 141 aa, H. influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 6 - protein, 149 aa, H. influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 7 - protein, 110 aa, H. influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 8 - protein, 275 aa, Artificial
  LVL735 (protein) (pelB sp)(ProtE aa 20-160) (GG)(PilA aa40-149)
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 9 - protein, 253 aa, Artificial
  PE-PilA fusion protein without signal peptide
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 10 - protein, 630 aa, Moraxalla catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 11 - protein, 520 aa, Artificial
  MC-001 (protein) - (M)(UspA2 amino acids 30 - 540)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 12 - protein, 512 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 13 - protein, 513 aa, Artificial
  MC-003 (Protein) - (M)(UspA2 amino acids 30-540)(H)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKH

SEQ ID NO: 14 - protein, 514 aa, Artificial
  MC-004 (Protein) - (M)(UspA2 amino acids 30-540)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKHH

SEQ ID NO: 15 - protein, 499 aa, Artificial
  MC-005 (Protein) - (M)(UspA2 amino acids 30-519)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSASHHHHHH

SEQ ID NO: 16 - protein, 491 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 17 - protein, 544 aa, Artificial
  MC-007 (Protein) - (M)(UspA2 amino acids 30-564)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAASHHHHHH

SEQ ID NO: 18 - protein, 538 aa, Artificial
  MC-008 (Protein) - (M)(UspA2 30-564)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 19 - protein, 537 aa, Artificial
  MC-009 (Protein) - (M)(UspA2 31-564)(HH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 20 - protein, 536 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 21 - protein, 519 aa, Artificial
  MC-011 (Protein) - (M)(UspA2 amino acids 31-540)(ASHHHHHH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 22 - protein, 613 aa, Moraxalla catarrhalis
MKTMKLLPLKIAVTSAMIIGLGAASTANAQSRDRSLEDIQDSISKLVQDDINTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 23 - protein, 644 aa, Moraxalla catarrhalis
MKTMKLLPLKIAVTSALIIGLGAASTANAQQQLQTETFLPNFLSNDNYDLTDPFYHNMILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 24 - protein, 591 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLESNVEEGLLDLSGRLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 25 - protein, 687 aa, Moraxalla catarrhalis
MKTMKLLPLKIAVTSAMIIGLGAASTANAQQQQQQQQQQQSRTEIFFPNIFFNENHDELDDAYHNIILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKGLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 26 - protein, 683 aa, Moraxalla catarrhalis
MKTMKLLPLKIAVTSAMIVGLGMASTANAQQQKSPKTETFLPNIFFNEYADDLDTLYHNMILGDTAITHDDQYKFYADDATEVPDSLFFNKILHDQLLYGFKEGDKIIPLDENGKPVYKLDKRLENGVQKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 27 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGGTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 28 - protein, 684 aa, Moraxalla catarrhalis
MKTMKLLPLKIAVTSAMIIGLGAASTANAQQQQKTKTEVFLPNLFDNDYYDLTDPLYHSMILGDTATLFDQQDNSKSQLKFYSNDKDSVPDSLLFSKLLHEQQLNGFKAGDTIIPLDKDGKPVYTQDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLENNIEEGLLELSGHLIDQKADLTKDIKTLENNIEEGLLELSGHLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 29 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKKGDTIIPLDENGKPVYKLDSIVEQGKTKTVYSVTTKTATADDVNSAYSRGIQGDIDDLYEANKENVNRLIEHGDKIFANEESVQYLNREVQNNIENIYELVQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 30 - protein, 574 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGHLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 31 - protein, 678 aa, Moraxalla catarrhalis
MKTMKLLPLKIAVTSALIVGLGAASTANAQQQPQTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 32 - protein, 678 aa, Moraxalla catarrhalis
MKTMKLLPLKIAVTSAMIVGLGAASTANAQQQQQPRTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKTLENNIEEGLLELSGHLIDQKADLTKDIKALESNVEEGLLDLSGRLLDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 33 - protein, 587 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLENNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDSRVTALDTKVNALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 34 - protein, 678 aa, Moraxalla catarrhalis; identical to SEQ ID NO: 32

SEQ ID NO: 35 - protein, 616 aa, Moraxalla catarrhalis
MKTMKLLPLKIAVTSALIVGLGAASTANAQVRDKSLEDIEALLGKIDISKLEKEKKQQTELQKYLLLSQYANVLTMEELNKNVEKNTNSIEALGYEIGWLENDIADLEEGVEELTKNQNTLIEKDEEHDRLIAQNQADIKTLENNVVEELFNLSDRLIDQEADIAKNNASIEELYDFDNEVAERIGEIHAYTEEVNKTLEKLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 36 - protein, 676 aa, Moraxalla catarrhalis
MKTMKLLPLKIAVTSALIVGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKALESNVEEGLLDLSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 37 - protein, 629 aa, Moraxalla catarrhalis
MKTMKLLPLKIAVTSALIVGLGAASTANAQNGTSTKLKNLKEYAQYLDNYAQYLDDDIDDLDKEVGELSQNIAKNQANIKDLNKKLSRDIDSLREDVYDNQYEIVNNQADIEKNQDDIKELENNVGKELLNLSGRLLDQKADIDNNINNIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 38 - protein, 683 aa, Moraxalla catarrhalis
MKTMKLLPLKIAVTSAMIVGLGMASTANAQQQRSPKTETFLPNIFFNEYADDLDTLYHNMILGDTAITHDDQYKFYADDATEVPDSLFFNKILHDQLLYGFKEGDKIIPLDENGKPVYKLDKRLDNGVQKTVYSVTTKTATADDVNSAYSRGIQGDIDDLYEANKENVNRLIEHGDKIFANEESVQYLNKEVQNNIENIHELAQQQDQHSSDIKTLKKNVEEGLLELSGHLIDQKTDIAQNQTDIQDLATYNELQDQYAQKQTEAIDALNKASSENTQNIAKNSNRIKALENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 39 - protein, 700 aa, Moraxalla catarrhalis
MKTMKLLPLKIAVTSAMIVGLGAASTANAQAQSNRSLDQVQALLRGIDETKIKKEIQQSQQPELNKYLTFNQLANALNIEELNNNVQKNTQRLDSAATLYGDLSKTVPKSIKENKESIKENKESIKENKESIKENKESIKENKESIKENKESITTLTRKSFQNQVDIVRNNASIEDLYAYGQEVAKSIGEIHAYTEEVNKTLENLITNSVENTNNITKNKADIQALENNVVEELFNLSGRLIDQKADIDNNINNIYELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTVIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 40 - protein, 676 aa, Moraxalla catarrhalis
MKTMKLLPLKIAVTSALIVGLGAASTANAQATETFLPNLFDNDYIETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 41 - protein, 678 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGRLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 42 - protein, 613 aa, Moraxalla catarrhalis; identical to SEQ ID NO: 22

SEQ ID NO: 43 - protein, 589 aa, Moraxalla catarrhalis
MKTMKLLPLKIAVTSALIVGLGAASTANAQATNKDITLEDVLKSIEEIDPYELRDYIEYPTAIERFLLLSQYGNTLTLEEFDNDIELLDQDVEDLEESVTELAKNQNSLIEQGEAIKEDLQGLADFVERQEDKILQNETSIKKNTQRNLVNGFEIEKNKDAIAKNNESIEDLYDFGHEVAKSIGEIHAHNEAQNETLKDLITNSVKNTDNITKNKADIQALESNVEKGLLELSGHLIDQKADIDNNINNIHELAQQQDQHSSDIKTLKKNVEEGLLELSGHLIDQKSDIAQNQANIQDLATYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 44 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGDTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 45 - protein, 650 aa, Moraxalla catarrhalis
MKTMKLLPLKIAVTSALIVGLGAASTANAQQQQKTKTEVFLPNLFYNDYIEETDLLYHNMILGDTAALVDRQNYSNSQLKFYSNDEESVPDSLLFSKMLNNQQLNGFKAGDIIIPVDANGQVIYQKDTRVEGGKTRTVLSVTTKIATQQDVDSAYSRGIQGKVNDLDDEMNFLNHDITSLYDVTANQQDDIKGLKKGVKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 46 - protein, 616 aa, Moraxalla catarrhalis
MKTMKLLPLKIAVTSALIVGLGAVSTTNAQAQSRSLDQIQTKLADLAGKIAAGKNGGGQNNQNNQNDINKYLFLSQYANILTMEELNNNVVKNSSSIETLETDFGWLENDVADLEDGVEELTKNQNTLIEKDEEHDRLIAQNQADIQTLENNVVEELFNLSDRLIDQKADIAKNQADIAQNNESIEELYDFDNEVAEKIGEIHAYTEEVNKTLQDLITNSVKNTDNIDKNKADIDNNINHIYELAQQQDQHSSDIKTLKNNVEEGLLELSGHLIDQKADLTKDIKTLENNVEEGLLDLSGRLIDQKADIAKNQADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 47 - protein, 668 aa, Moraxalla catarrhalis
MKTMKLLPLKIAVTSALIVGLGTASTANAQVASPANQKIQQKIKKVRKELRQDIKSLRNDIDSNTADIGSLNDDVADNQDDILDNQADIAKNQDDIEKNQADIKELDKEVGVLSREIGSLNDDIADNYTDIIDNYTDIIDNQANIAKNQDDIEKNQADIKELDKEVGVLSREIGSLNDDVADNQDDIAKNQADIQTLENNVEEGLLELSGHLLDQKADIDNNINNIYELAQQQDQHSSDIKTLKKNVEEGLLELSGHLIDQKTDIAQNQANIQDLATYNELQDQYAQEQTEAIDALNKASSENTQNIAKNSNRIKALESNVEEGLLELSGHLIDQKADLTKDIKALESNVEEGLLELSGHLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKVSADTKFAATADAITKNGNAITKNAKSITDLGTKVDAFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 48 - protein, 674 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 49 - protein, 613 aa, Moraxalla catarrhalis
MKTMKLLPLKIAVTSAMIIGLGAASTANAQSRDRSLEDIQDSISKLVQDDIDTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 50 - protein, 576 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 51 - protein, 684 aa, Moraxalla catarrhalis; identical to SEQ ID NO: 44

SEQ ID NO: 52 - protein, 686 aa, Moraxalla catarrhalis
MKTMKLLPLKIAVTSAMIIGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 53 - protein, 630 aa, Moraxalla catarrhalis; identical to SEQ ID NO: 10

SEQ ID NO: 54 - protein, 616 aa, Moraxalla catarrhalis
MKTMKLLPLKIAVTSALIVGLGAASTANAQAQDRSLEQIQDKLANLVEKIEQAKSQNGQSQKDINQYLLLSQYANVLTMEELNNNVVKNSSSIETLDNDIAWLNDDLIDLDKEVGVLSRDIGSLHDDVAQNQADIKTLKNNVVEELFNLSDRLIDQEADIAQNNESIEDLYDFGREVAESIGEIHAHNEAQNETLKDLITNSVKNTDNITKNKADIQALENDVGKELLNLSGRLIDQKADIDNNINHIYELAQQQDQHSSDIKTLKNNVEEGLLELSGHLIDQKADLTKDIKALESNVEEGLLDLSGRLLDQKADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 55 - protein, 614 aa, Moraxalla catarrhalis
MKTMKLLPLKIAVTSAMIIGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 56 - protein, 679 aa, Moraxalla catarrhalis
MKTMKLPPLKIAVTSAMIIGLGAASTANAQTTETFLPNLFDNDYTETTDPLYHGMILGDTAITQDTQYKFYAENGNEVPDSLFFNKILHDQLLNGFKAGDTIIPLDENGKPVYKLDERTENGVKRKVYSVTTKTATQADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEECLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 57 - protein, 724 aa, Moraxalla catarrhalis
MKTMKLLPLKIAVTSALIVGLGAASTANAQETLEEVLESIKQINEQDLQDDIGYNSALDRYLVLSQYGNLLIAKELNENVEKNSNSIAKNSNSIADLEADVGYLAENQNTLIEQNETINQELEGITHELESFIAYAHAQDQKNLVNEFEIEKNKDAIAKNNESIEDLYDFGHEVAESIGEIHAYTEEVNKTLENLITNSVKNTDNITKNKADIQALESNVEKELLNLSGRLIDQKADIDNNINHIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 58 - protein, 611 aa, Moraxalla catarrhalis
MKTMKLLPLKIAVTSALIVGLGAASTANAQAQARDRSLEDIQALIGNIDVDKIRSQKQKNPEIFQYLLLNQLSNTLITDELNNNVIKNTNSIETLDNDIAWLNDDLIDLDKEVGVLSRDIGSLHDDVAQNQADIKTLENNVVEELFNLSDRLIDQEAEIAQNNESIEDLYDFGREVAESIGEIHAHNEAQNETLKDLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF